(19) United States
(12) Patent Application Publication
(10) Pub. No.: US 2024/0024405 A1
(43) Pub. Date: Jan. 25, 2024

(54) COMPOSITIONS OF CASPASE INHIBITORS AND METHODS OF USE THEREOF

(71) Applicants: The University of Hong Kong, Hong Kong (CN); Centre for Virology, Vaccinology and Therapeutics Limited, Hong Kong (CN)

(72) Inventors: Hin Chu, Hong Kong (CN); Fuk-Woo Jasper Chan, Hong Kong (CN); Kwok-Yung Yuen, Hong Kong (CN)

(21) Appl. No.: 18/350,402

(22) Filed: Jul. 11, 2023

Related U.S. Application Data
(60) Provisional application No. 63/389,693, filed on Jul. 15, 2022.

(51) Int. Cl. A61K 38/00; A61P 31/14

(52) U.S. Cl. CPC A61K 38/005; A61P 31/14

(57) ABSTRACT

Methods and compositions for treating a coronavirus infection in a subject are provided. Also provided are methods and compositions for preventing or treating a coronavirus-associated disease in a subject. The methods and compositions include administering a pharmaceutical composition containing an effective amount of a caspase-6 inhibitor to a subject. The amount of the caspase-6 inhibitor contained in the pharmaceutical composition is effective to reduce replication of the coronavirus in the subject. The disclosed methods and compositions ameliorate lung pathology associated with the coronavirus infection and increases survival in the subject.

### CROSS REFERENCE TO RELATED APPLICATIONS

This application claims benefit of and priority to U.S. Provisional Application No. 63/389,693, filed Jul. 15, 2022, which is specifically incorporated by reference herein in its entirety.

### REFERENCE TO THE SEQUENCE LISTING

The Sequence Listing submitted as a text file named “UHK_01211_US_ST26.xml” created on Jun. 30, 2023, and having a size of 114,045 bytes is hereby incorporated by reference pursuant to 37 C.F.R. § 1.834(c)(1).

### FIELD OF THE INVENTION

The disclosed invention is generally in the field of antiviral agents and specifically in the area of compositions and methods for treatment of coronavirus infections.

### BACKGROUND OF THE INVENTION

Seven coronaviruses are known to infect humans. Among them, three highly pathogenic coronaviruses, including severe acute respiratory syndrome coronavirus 2 (SARS-CoV-2) (Chan, J. F. et al. (2020) Lancet Vol. 395(10223), pages 514-523, doi:10.1016/S0140-6736(20)30154-9; Zhou, P. et al. (2020) Nature Vol. 579(7836), pages 270-273, doi:10.1038/s41586-020-2012-7), Middle East respiratory syndrome coronavirus (MERS-CoV) (Zaki, A. M., et al. (2012) New England Journal ofMedicine Vol. 367, pages 1814-1820, doi:10.1056/NEJMoa1211721; Chan, J. F. et al. (2015) Clinical Microbiology Reviews Vol. 28(2), pages 465-522, doi:10.1128/CMR.00102-14), and SARS-CoV-15, have emerged over the last two decades and have dramatically impacted the global public health (To, K. K. et al. (2021) Emerging Microbes and Infections Vol. 10(1): pages 507-535, doi:10.1080/22221751.2021.189829). These highly pathogenic coronaviruses vary in their transmissibility and pathogenicity, and their underlying mechanisms remain largely unexplained (Li, X., et al. (2020) Current Opinions in HIV and AIDS Vol. 15(6), pages 328-335, doi:10.1097/COH.000000000000 0650). A common feature of these coronaviruses is their propensity to induce apoptosis in the infected target cells. Previous studies have shown that SARS-CoV-1 infection induced apoptosis in cell culture and infected patients (Chau, T. N. et al. (2004) Hepatology Vol. 39(2), pages 302-310, doi:10.1002/hep.20111; Ding, Y. et al. (2003) Journal of Pathology Vol. 200(3), pages 282-289, doi:10.1002/path.1440). Previous studies have shown that MERS-CoV triggered severe apoptosis in lung epithelial cells (Tao, X. et al. (2013) Journal of Virology Vol. 87(17), pages 9953-9958, doi:10.1128/JVI.01562-13), primary T cells (Chu, H. et al. (2016) Journal of Infectious Diseases Vol. 213(6), pages 904-914, doi:10.1093/infdis/jiv380), and in vivo infected animals tissues (Yeung, M. L. et al. (2016) Nature Microbiology Vol. 1(3), 16004, doi:10.1038/nmicrobiol.2016.4; Chu, H. et al. (2021) Science Advances Vol. 7(25), doi:10.1126/sciadv.abf8577). More recently, SARS-CoV-2-induced apoptosis was similarly documented in infected human tracheobronchial epithelial cells (Zhu, N. et al. (2020) Nature Communications Vol. 11(1), Article 3910, doi:10.1038/s41467-020-17796-z), lungs of infected hamsters (Chan, J. F. et al. (2020) Clinical Infectious Diseases Vol. 71(9), pages 2428-2446, doi:10.1093/cid/ciaa325), and lung specimens of Coronavirus Disease 2019 (COVID-19) patients (Li, S. et al. (2020) JCI Insight Vol. 5(12), Article e138070 doi:10.1172/jci.insight.138070). Thus, there is a need for highly effective antivirals against the infection of highly pathogenic coronaviruses such as for example SARS-CoV-2 and MERS-CoV and variants thereof.

Therefore, it is an object of the invention to provide compositions and methods of use thereof, for the treatment of coronavirus infections by highly selective, controllable inhibition of caspase-6 signaling.

It is another object of the invention to provide compositions and methods of use thereof, for the prevention and/or treatment of coronavirus-associated diseases by highly selective, controllable inhibition of caspase-6 signaling.

### BRIEF SUMMARY OF THE INVENTION

It has been established that administration of a caspase-6 inhibitor to a subject with a coronavirus infection reduces the replication of the coronavirus in the subject. It has further been established that administration of a caspase-6 inhibitor to a subject with a coronavirus infection, ameliorates lung pathology associated with the coronavirus infection and increases survival in the subject. It has been demonstrated that coronaviruses, e.g., SARS-CoV-2, activate necroptosis in addition to apoptosis (Li et al., Signal Transduct Target Ther., 5(1):235 (2020). Necroptosis is a more inflammatory form of cell death when compared to apoptosis. It is likely that inhibition of caspase-8 signaling inhibits necroptosis; thus, using a pan-caspase inhibitor will likely exacerbate necroptosis in a coronavirus infected host, and increase the severity of the coronavirus infection. Selective caspase-6 inhibition does not affect signaling of caspase-8; thereby, having little to no effect on necroptosis. Thus, selective inhibition of caspase-6 signaling is likely more effective at reducing the severity of a coronavirus infection.

Disclosed are methods of treating a coronavirus infection in a subject including administering a pharmaceutical composition containing an effective amount of a caspase-6 inhibitor to the subject in need thereof, wherein the amount of the caspase-6 inhibitor in the pharmaceutical composition when administered to the subject in need thereof, is effective to reduce replication of the coronavirus in the subject.

Also disclosed are methods of preventing or treating a coronavirus-associated disease in a subject including administering to a subject, a pharmaceutical composition containing an effective amount of a caspase-6 inhibitor to the subject in need thereof, wherein the amount of the caspase-6 inhibitor in the pharmaceutical composition when administered to the subject in need thereof, is effective to reduce replication of the coronavirus in the subject.

Typically, the amount of the caspase-6 inhibitor in the pharmaceutical composition is effective, when administered to the subject, to reduce the replication of the coronavirus in the subject compared to an untreated subject with a coronavirus infection. In some forms, the caspase-6 inhibitor contained in the pharmaceutical composition also inhibits one or more other caspases.

In some forms, the caspase-6 inhibitor contained in the pharmaceutical composition is a pan caspase inhibitor. In some forms, the caspase-6 inhibitor contained in the pharmaceutical composition is Z-VEID-FMK or Ac-VEID-CHO. In preferred forms, the caspase-6 inhibitor contained in the pharmaceutical composition is Z-VEID-FMK.

In other forms, the caspase-6 inhibitor contained in the pharmaceutical composition is a small molecule. In some forms, caspase-6 inhibitor contained in the pharmaceutical composition is a nucleic acid molecule selected from the group comprising a single stranded antisense nucleic acid (ssRNA), a small interfering NA (siRNA), a short hairpin RNA (shRNA), and a microRNA (miRNA).

Typically, the amount of the caspase-6 inhibitor in the pharmaceutical composition is effective, when administered to the subject, to reduce coronavirus replication by 50% or more 24 hours following administration. In some forms, the pharmaceutical composition is effective, when administered to the subject, to deliver the caspase-6 inhibitor at a dose from about 7.5 mg/kg/day or more. In some forms, the pharmaceutical composition is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof following the detection of the coronavirus in the subject. In some forms, the pharmaceutical composition is administered via oral, intranasal, intraperitoneal, or intrathecal administration.

In preferred forms, the subject is a human. In some forms, the subject is immunocompromised. In some forms, the subject has a coronavirus-induced pneumonia, coronavirus-induced bronchitis, acute respiratory distress syndrome (ARDS), acute lung injury (ALI), multisystem inflammatory syndrome in children (MIS-C), and/or multisystem inflammatory syndrome in adults (MIS-A).

Typically, the coronavirus infection to be treated can be caused by an alpha coronavirus or beta coronavirus. In some forms, the coronavirus can be any form or variant of Human Coronavirus 229E (HCoV-229E), Human Coronavirus OC43 (HCoV-OC43), Human Coronavirus NL63 (HCoV-NL63), Human Coronavirus HKU1 (HCoV-HKU1), Middle East Respiratory Syndrome Coronavirus (MERS-CoV), Severe Acute Respiratory Syndrome Coronavirus 1 (SARS-CoV-1), and/or SARS-CoV-2. In some forms, the coronavirus can be Human Coronavirus 229E (HCoV-229E), Human Coronavirus OC43 (HCoV-OC43), Human Coronavirus NL63 (HCoV-NL63), Human Coronavirus HKU1 (HCoV-HKU1), Middle East Respiratory Syndrome Coronavirus (MERS-CoV), Severe Acute Respiratory Syndrome Coronavirus 1 (SARS-CoV-1), and/or SARS-CoV-2. In preferred forms, the coronavirus is a pathogenic coronavirus.

Preferably, the pathogenic coronavirus can be any form or variant of Middle East Respiratory Syndrome Coronavirus (MERS-CoV), Severe Acute Respiratory Syndrome Coronavirus 1 (SARS-CoV-1), and/or SARS-CoV-2. In some forms, the pathogenic coronavirus can be Middle East Respiratory Syndrome Coronavirus (MERS-CoV), Severe Acute Respiratory Syndrome Coronavirus 1 (SARS-CoV-1), and/or SARS-CoV-2.

In some forms, when the pathogenic coronavirus is a SARS-CoV-2 variant, the SARS-CoV-2 variant can be any form or variant of the alpha variant, beta variant, gamma variant, delta variant, epsilon variant, eta variant, iota variant, kappa variant, mu variant, omicron variant, zeta variant, 1.617.3 variant and/or lambda variant. In some forms, when the pathogenic coronavirus is a SARS-CoV-2 variant, the SARS-CoV-2 variant can be the alpha variant, beta variant, gamma variant, delta variant, epsilon variant, eta variant, iota variant, kappa variant, mu variant, omicron variant, zeta variant, 1.617.3 variant and/or lambda variant. In some forms, when the pathogenic coronavirus is SARS-CoV-2, the SARS-CoV-2 variant can be a variant of the wild-type strain of the coronavirus. “Wild-type” as used herein, refers to the original strain of coronavirus considered to be the background strain of the coronavirus containing no major mutations.

The coronavirus infection to be treated may be caused by an alpha coronavirus or beta coronavirus that infects a non-human mammal. In some forms, the alpha coronavirus or beta coronavirus is a variant derived from bat coronavirus belonging to the family of alpha coronavirus or beta coronavirus. In some forms, the coronavirus infection to be treated is caused by a gamma coronavirus or a delta coronavirus.

Also disclosed are pharmaceutical compositions for the treatment of a coronavirus infection in a subject containing an effective amount of a caspase-6 inhibitor, wherein the amount of caspase-6 inhibitor in the composition is effective, when administered to the subject, to reduce replication of coronavirus in the subject. Typically, the amount of the caspase-6 inhibitor in the pharmaceutical composition is effective, when administered to the subject, to reduce replication of coronavirus in the subject compared to an untreated subject with a coronavirus infection.

In some forms, the pharmaceutical composition containing the caspase-6 inhibitor also inhibits one or more other caspases. In some forms, the caspase-6 inhibitor is a pan caspase inhibitor. In some forms, the caspase inhibitor is Z-VIED-FMK or Ac-VEID CHO. In preferred forms, the caspase inhibitor is Z-VIED-FMK.

### BRIEF DESCRIPTION OF THE DRAWINGS

FIG. 1 is a schematic of caspase-6 knock out (KO) in the human dipeptidyl peptidase 4 Knock-In (hDPP4 KI) mice.

FIGS. 2A-2F are scatterplots of the gating strategy for the flow cytometry experiments. The BEAS2B cell population was gated with SSC-A vs FSC-A. Most cells except for the cell debris at the lower left corner were gated. A mock-infected sample treated with the apoptosis inhibitor, z-VAD-fmk, was used as the gating control for active caspase-3-positive cells. A mock-infected sample treated with DMSO was used as the gating control for MERS-CoV-N-positive cells.

FIGS. 3A-3E are bar graphs of results demonstrating that caspase-6 inhibition limits coronavirus replication. FIGS. 3A-3E illustrate the replication of human-pathogenic coronaviruses treated with 100M z-VAD-fnk or DMSO. Samples were harvested at 24 hpi (hours post infection) and viral gene expression was quantified with RT-qPCR (n=3). FIGS. 3F-3I are scatter dot plots of results showing caspase-6 inhibition reduces MERS-CoV replication in different cell types. BEAS2B cells (FIG. 3F), Calu3 cells (FIG. 3G), Huh7 cells (FIG. 3H), and BSC1 cells (FIG. 3I) were treated with z-VAD-fmk at the indicated concentrations. Cell viability was quantified with CellTiter-Glo assays at 24 hours post treatment (n=6).

FIGS. 4A-4J are scatterplots of flow cytometry results of MERS-CoV-infected BEAS2B cells treated with 75 μM specific caspase inhibitors. Cells were fixed at 24 hpi. and labelled with the MERS-CoV N immune serum and an active caspase-3 antibody (n=3; DMSO (FIG. 4A); Caspase-1i, z-WEHD-fmk (FIG. 4B); Caspase-2i, z-VDVAD-fmk (FIG. 4C); Caspase-3i, z-DEVD-fmk (FIG. 4D); Caspase-4i, z-YVAD-fmk (FIG. 4E); Caspase-6i, z-VEID-fmk (FIG. 4F); Caspase-8i, z-IETD-fnk (FIG. 4G); Caspase-9i, z-LEHD-fmk (FIG. 4H); Caspase-10i, z-AEVD-fmk (FIG. 4I); and Mock-infected (FIG. 4J). FIG. 4K is a bar graph showing the quantification of the data for all specific caspase inhibitors represented in FIGS. 4A-4J.

FIGS. 5A-5E are bar graphs of the MDM, Caco2, Calu3, A549, and VeroE6 cells infected with MERS-CoV at 1 MOI and were treated with z-VEID-fmk, z-VAD-fmk, or DMSO. Cell lysate and supernatant samples were harvested at 24 hpi. MERS-CoV N gene copy was quantified with RT-qPCR.

FIGS. 6A-6G are bar graphs of results showing virus replication of MERS-CoV (BEAS2B; FIG. 6A), SARS-CoV-2 (Calu3; FIG. 6B), SARS-CoV-1 (Huh7; FIG. 6C), HCoV-229E (Huh7; FIG. 6D), HCoV-OC43 (BSC1; FIG. 6E), H1N1 (A549; FIG. 6F), and EV-71 (RD; FIG. 6G) with or without 100 μM z-VEID-fmk. Virus gene copy was quantified with RT-qPCR against the N gene (MERS-CoV, SARS-CoV-1, HCoV-229E, HCoV-OC43), RdRp gene (SARS-CoV-2), M gene (H1N1), or VP1 gene (EV-71). FIGS. 6H-6K are bar graphs of results showing Huh7 cells infected with SARS-CoV-2 Omicron BA.1 (FIG. 6H and FIG. 6I) or BA.2 (FIG. 6J and FIG. 6K) at 1 MOI and treated with z-VEID-fmk at the indicated concentrations. Cell lysate and supernatant samples were harvested at 24 hpi. SARS-CoV-2 RdRp gene copy was quantified with RT-qPCR (n=3) and infectious titer was quantified with TCID50 assays (n=3).

FIGS. 7A-7J are bar graphs of results showing the half-maximal inhibitory concentrations (IC50s) of z-VEID-fmk on the replication of MERS-CoV (HFL; FIG. 7A and FIG. 7F), SARS-CoV-2 (Calu3; FIG. 7B and FIG. 7G), SARS-CoV-1 (Huh7; FIG. 7C and FIG. 7H), HCoV-229E (Huh7; FIG. 7D and FIG. 7I) and HCoV-OC43 (BSC1; FIG. 7E and FIG. 7J) in cell lysate and supernatant samples were determined with RT-qPCR and TCID50 assays, respectively (n=4 for HCoV-OC43 and EV-71, n=3 for other viruses). FIGS. 7K-7O are dot plots of results from HFL cells (FIG. 7K), Calu3 cells (FIG. 7L), Huh7 cells (FIGS. 7M and 7N), and BSC1 cells (FIG. 7O) with or without infection with the indicated coronaviruses treated with z-VEID-fmk. Cell viability was quantified with CellTiter-Glo assays at 24 hours post treatment (n=3). Data represented mean and standard deviations from the indicated number of biological repeats.

FIGS. 8A-8D are bar graphs of results showing that caspase-6 inhibition attenuates MERS-CoV replication in human lung tissues and human intestinal organoids.

FIG. 8A is a bar graph of RT-qPCR results from the infection of ex vivo human lung tissues with MERS-CoV and treatment with z-VEID-fmk. In FIGS. 8B-8D, human intestinal organoids were infected with MERS-CoV and treated with z-VEID-fnk. FIG. 8B shows the quantification of N protein expression in organoids that were fixed at 24 hpi for immunostaining. FIG. 8C shows the percentage of infected cells per organoids was calculated from counting the number of infected cells and uninfected cells per organoid (n=5). Shown is N gene expression at the indicated time points was quantified with RT-qPCR (n=3) FIG. 8D shows the infectious titer as determined with plaque assays (n=6).

FIGS. 9A and 9B are graphical representations showing the conservation of the z-VEID-fmk binding pocket in human, mouse, and golden Syrian hamster caspase-6. FIG. 9A illustrates a multiple sequence alignment of human (Homo Sapiens, SEQ ID NO:53), mouse (Mus musculus, SEQ ID NO:54), and golden Syrian hamster (Mesocricetus auratus, SEQ ID NO:55) caspase-6 full-length sequences. The indices were labelled according to human caspase 6. VEID binding sites on caspase-6 were indicated with orange triangles. FIG. 9B is a molecular model of the VEID binding mode represented in 3D structure. Binding sites on caspase-6 and VEID were shown in green and magenta sticks, respectively. The binding site residues were labelled in red.

FIG. 10A is a schematic of the experimental timeline showing hDPP4 KI mice were intranasally inoculated with 2.5×10³ PFU MERS-CoV MA followed by intraperitoneal administration of 12.5 mg/kg/day z-VEID-fmk or DMSO for 6 days or until sample harvest. A subset of mice was harvested at day 2 and day 4 post infection.

Mouse lungs were immunolabelled to detect MERS-CoV N expression. FIGS. 10B and 10C are bar graphs of the viral gene expression in mouse lungs quantified with RT-qPCR (n=6). FIGS. 10D and 10E are bar graphs of the infectious titer as determined with plaque assays (n=6). FIGS. 10F-10I are bar graphs of the expression of pro-inflammatory cytokines and chemokines as quantified with RT-qPCR (n=6). Expression of TNFα (FIGS. 10F and 10G) and IP10 (FIGS. 10H and 10I) are shown.

FIGS. 11A-11H are bar graphs of results showing that caspase-6 inhibition reduces the expression of pro-inflammatory cytokines and chemokines in the lungs of hDPP4 KI mice. hDPP4 KI mice were intranasally inoculated with 2.5×10³ PFU MERS-CoV MA followed by intraperitoneal administration of 12.5 mg/kg/day z-VEID-fmk or DMSO for 6 days or until sample harvest. Mouse lungs were harvested at day 2 and day 4 post infection. Expression of pro-inflammatory cytokines and chemokines were quantified with RT-qPCR. Expression of IL6 at day 2 (FIG. 11A) and day 4 (FIG. 11B); MCP1 at day 2 (FIG. 11C) and day 4 (FIG. 11D); CXCL5 at day 2 (FIG. 11E) and day 4 (FIG. 11F); CXCL9 at day 2 (FIG. 11G) and day 4 (FIG. 11H) are shown. Data represented mean and standard deviations from the indicated number of biological repeats.

FIGS. 12A and 12B are body weight (FIG. 12A) and survival (FIG. 12B) curves of the infected mice monitored for 14 days. Data represented mean and standard deviations from the indicated number of biological repeats.

FIG. 13A is a schematic of the experimental timeline. Golden Syrian hamsters were intranasally inoculated with 3×10³ PFU SARS-CoV-2 followed by intraperitoneal administration of 12.5 mg/kg/day z-VEID-fnk or DMSO for 4 days. FIGS. 13B and 13C are bar graphs of quantification of the viral gene copy (FIG. 13B) and infectious titer (FIG. 13C) from hamster lungs at day 4 post infection as measured by RT-qPCR and TCID₅₀ assays, respectively. FIGS. 13D-13J are bar graphs of the expression of pro-inflammatory cytokines and chemokines was quantified with RT-qPCR (n=6). Expression of TNFα (FIG. 13D), IL6 (FIG. 13E), IL1β (FIG. 13F), IFNγ (FIG. 13G), IP10 (FIG. 13H), CXCL3 (FIG. 13I), and CXCL5 (FIG. 13J) are shown. FIG. 13K is a line graph of the body weight change of SARS-CoV-2-infected hamsters administered z-VEID-fmk or mock treatment as documented from day 0 to day 4 post infection. FIGS. 13L-13O are bar graphs showing the quantitative scores for the lung histopathological changes of SARS-CoV-2-infected hamsters with or without z-VEID-fmk treatment. Three categories of characteristic histopathological changes including bronchiolitis (FIG. 13L), alveolitis (FIG. 13M) and vasculitis (FIG. 13N) were examined and scored. (n=6 and two-three lung lobes were examined from each hamster). FIG. 13O illustrates the total pathology score. Data represented mean and standard deviations from the indicated number of biological repeats.

FIG. 14A is a bar graph of the MERS-CoV N gene copies in the supernatant as determined with RT-qPCR at 24 hpi (n=4). MERS-CoV-infected BEAS2B cells were incubated with z-VEID-fnk in a time of addition assay. FIGS. 14B and 14D-14G are bar graphs of results from experiments in which caspase-6 stable knockdown A549 and BEAS2B cells were infected with MERS-CoV at 0.1 MOI. FIG. 4B illustrate RT-qPCR quantification against the MERS-CoV N gene for virus entry in cell lysates harvested at 1 hpi (n=4). In FIGS. 14D-14G illustrate RT-qPCR quantification of against the MERS-CoV N gene for virus replication at 24 hpi (n=4). FIG. 14C is a bar graph of the caspase-6 RNA (n=3) and protein expression from caspase-6 stable knockdown A549 and BEAS2B cells. FIGS. 14I-14L are bar graphs showing RT-qPCR quantification against the MERS-CoV N gene (n=3) in BEAS2B (FIGS. 141 and 14J) and A549 (FIGS. 14K and 14L) cell lysate and supernatant samples harvested at 24 hpi. Caspase-6 stable knockdown or scrambled knockdown A549 and BEAS2B cells were infected with MERS-CoV at 0.1 MOI followed by treating the cells with z-VEID-fmk at the indicated concentrations. FIGS. 14M and 14N are bar graphs showing the quantified MERS-CoV N gene copy at 24 hpi (n=4). MDMs were treated with caspase-6 or nontargeting siRNA and infected with MERS-CoV. FIGS. 14O and 14P are dot plots showing caspase-6 KO or scrambled KO Huh7 cells infected with MERS-CoV at 1 MOI. Cell lysate and supernatant samples were harvested at 24 hpi and 48 hpi to quantify virus replication with RT-qPCR against the MERS-CoV N gene (FIG. 14O) or with TCID50 assays (FIG. 14P) (n=4). FIGS. 14Q and 14R are bar graphs of results from the infection of caspase-6- or caspase-3-overexpressed 293T cells with MERS-CoV at 1 MOI. MERS-CoV replication was quantified at 1 and 24 hpi with RT-qPCR against the MERS-CoV N gene (n=4) in cell lysates (FIG. 14Q) and supernatant (FIG. 14R) samples.

FIG. 15A is a schematic of the experimental timeline. hDPP4 KI/caspase-6 KO and hDPP4 KI/caspase-6 WT mice were intranasally inoculated with 2.5×10³ PFU MERS-CoV MA. Mouse lung samples were harvested at day 2 and day 4 post infection. FIG. 15B is a bar graph of the MERS-CoV-N gene expression in mouse lungs as quantified with RT-qPCR (n=7). FIG. 15C is a bar graph of the MERS-CoV-N infectious titer as determined with plaque assays (n=7).

FIG. 16 is a dot plot of results showing that caspase-6 is activated by apoptosis triggered by MERS-CoV infection or Staurosporine (STS) stimulation. Huh7 cells were infected with MERS-CoV at 1 MOI for 12 hours. In parallel, Huh7 cells were stimulated with STS at 1 μM for 6 hours. Caspase-6 activity in the cell lysate was determined with the caspase-Glo-6 assay kit (n=4).

FIG. 17A is a bar graph of results showing that caspase-6 mediated N-cleavage modulates IFN signaling. Huh7 cells were pretreated with DMSO, 5 μM Filgotinib, 5 μM Ruxolitinib, or 5 μM IFN alpha-IFNAR-IN-1 hydrochloride for 1 h and infected with MERS-CoV at 1 MOI. After infection, the cells were incubated in media supplemented with DMSO, 5 μM Filgotinib, 5 μM Ruxolitinib, or 5 μM IFN alpha-IFNAR-IN-1 hydrochloride in the presence of z-VEID-fmk. Cell lysates were harvested at 24 hpi. MERS-CoV N gene copy was quantified with RT-qPCR (n=3). FIGS. 17B and 17D are bar graphs of results from transfecting 293T cells with an IFN-β-Luc reporter plasmid, expression constructs of MERS-CoV N or E and caspase-6 or caspase-3, with or without poly(I:C). Cells were incubated for 24 hours before harvesting for dual-luciferase reporter assays (n=4). FIG. 17C is a bar graph of results from transfecting 293T cells with an IFN-β-Luc reporter plasmid, expression construct of MERS-CoV N with or without poly(I:C). Cells were incubated for 24 hours before harvesting for dual-luciferase reporter assays (n=3). FIGS. 17E-17G are bar graphs of results from transfecting 293T cells with the same set of plasmids. Gene expression of IFN-β (FIG. 17E), IFIT3 (FIG. 17F), and OAS1 (FIG. 17G) was quantified with RT-qPCR (n=6 for IFN-β and IFIT1, n=4 for OAS1). FIGS. 17H-17K are bar graphs of results from transfecting 293T cells with an IFN-β-Luc reporter plasmid, expression constructs of MERS-CoV N or E and caspase-6 or caspase-3, with or without poly(I:C). Gene expression of IFIT1 (FIG. 17H), IFIT2 (FIG. 17I), IFITM3 (FIG. 17J), and TRIM22 (FIG. 17K) was quantified with RT-qPCR (n=6 for IFIT1 and IFIT2, n=4 for IFITM3 and TRIM22). FIG. 17L is a bar graph of results from transfecting 293T cells with an IFN-β-Luc reporter plasmid, expression constructs of caspase-6 and MERS-CoV N, ORF4a, ORF4b, or M, with or without poly(I:C). Cells were incubated for 24 hours before harvesting for dual-luciferase reporter assays (n=3). FIG. 17M is a bar graph of results from transfecting 293T cells with an IFN-β-Luc reporter plasmid, expression constructs of caspase-6 and MERS-CoV N, ORF4a, ORF4b, or M, with or without poly(I:C). Cells were incubated for 24 hours before harvesting for dual-luciferase reporter assays (n=3 for empty vector control and poly(I:C) only; n=7 for all other groups). FIG. 17N is bar graph of results from transfecting 293T cells with an IFN-β-Luc reporter plasmid, expression constructs of caspase-6 and coronavirus N, with or without poly(I:C). Cells were incubated for 24 hours before harvesting for dual-luciferase reporter assays (n=3). FIGS. 17O and 17P are bar graphs of results from transfecting 293T cells with the same set of plasmids. Gene expression of IFIT3 (FIG. 17O) and OAS1 (FIG. 17P) was quantified with RT-qPCR (n=4). Data represented mean and standard deviations from the indicated number of biological repeats.

FIG. 18A is a schematic of N mutants. FIG. 18B is a bar graph of results from transfecting 293T cells with an IFN-3-Luc reporter plasmid, expression constructs of caspase-6 and MERS-CoV N or N mutants, with or without poly(I:C). Cells were incubated for 24 hours before harvesting for dual-luciferase reporter assays (n=3). FIGS. 18C and 18D are bar graphs of results from transfecting 293T cells were transfected with an IFN-β-Luc reporter plasmid, expression constructs of caspase-6 and MERS-CoV N, N(1-241), or N(242-413), with or without poly(I:C). Cells were incubated for 24 hours before harvesting for dual-luciferase reporter assays (n=6) (FIG. 18C) or IFN-β ELISA (n=4) (FIG. 18D). FIG. 18E is a bar graph of results from transfecting Huh7 cells with an IFN-β-Luc reporter plasmid, expression constructs of caspase-6, MERS-CoV N, N(1-241), or N(242-413), with or without poly(I:C). Cells were incubated for 24 hours before harvesting for dual-luciferase reporter assays (n=3). FIG. 18F is a schematic of SARS-CoV-2 N mutants. FIG. 18G and FIG. 18H are bar graphs of results from transfecting 293T (FIG. 18G) and Huh7 (FIG. 18H) cells with an IFN-β-Luc reporter plasmid, expression constructs of caspase-6 and SARS-CoV-2 N, N(1-215), N(216-419), with or without poly(I:C). Cells were incubated for 24 hours before harvesting for dual-luciferase reporter assays. n=5 for 293T cells. For Huh7 cells, n=3 for the empty vector, poly(I:C) only, and poly(I:C)+caspase-6 groups; n=6 for all other groups. FIG. 18I is a bar graph of results from transfecting 293T cells with an IFN-β-Luc reporter plasmid, expression constructs of caspase-6, SARS-CoV-2 N, NSP13, NSP15, ORF6, ORF8, or ORF3b, with or without poly(I:C). Cells were incubated for 24 hours before harvesting for dual-luciferase reporter assays (n=3 for the empty vector and poly(I:C) only groups; n=7 for all other groups). Data represented mean and standard deviations from the indicated number of biological repeats.

FIGS. 19A-19C are bar graphs of results from transfecting 293T cells with an IFN-β-Luc reporter plasmid and expression constructs of MERS-CoV N(1-241) or N(242-413), with or without RIG-IN (FIG. 19A), MAVS (FIG. 19B), or TBK1 (FIG. 19C) plasmids. Cells were incubated for 24 hours before harvesting for dual-luciferase reporter assays (n=4). Data represented mean and standard deviations from the indicated number of biological repeats. FIG. 19D is a dot plot showing the Pearson correlation coefficient between MERS-CoV N fragments and IRF3 in Huh7 cells as quantified with the Zeiss ZEN software (n=5). Huh7 cells were transfected with expression constructs of IRF3, MERS-CoV N, N(1-241), or N(242-413), and poly(I:C). Cells were fixed at 24 hours post transfection. Localization of N was detected with an in-house guinea pig anti-N immune serum and IRF3 was detected with a rabbit anti-HA antibody. Cell nuclei were identified with the DAPI stain. Data represented mean and standard deviations from the indicated number of biological repeats.

FIGS. 20A and 20B are sequence chromatograms representing construction of rMERS-CoV/TKKA by introducing a ‘D’ to ‘A’ change at the TKKD motif of the MERS-CoV N gene with red recombineering. FIGS. 20C-20E and FIGS. 20F-20H are bar graphs of results from infecting A549 (FIG. 20C and FIG. 20F), Huh7 (FIG. 20D and FIG. 20G), and VeroE6 (FIG. 20E and FIG. 20H) cells with rMERS-CoV/TKKA or rMERS-CoV/TKKD at 1 MOI (A549 and Huh7) or 0.5 MOI (VeroE6). MERS-CoV N gene copies in the cell lysate samples at the indicated time points were quantified with RT-qPCR (n=3) (FIGS. 20C-20E) and infectious titers at the indicated time points were quantified with TCID50 assays (n=3) (FIGS. 20F-20H). FIGS. 20I-20N are bar graphs of results from infecting Huh7 (FIGS. 20I-20K) and A549 (FIGS. 20L-20N) cells with rMERS-CoV/TKKA or rMERS-CoV/TKKD and treated with z-VEID-fmk at the indicated concentrations. MERS-CoV N gene copies were quantified with RT-qPCR (n=3). FIGS. 20O-20T are bar graphs of results from infecting Huh7 (FIGS. 20O-20Q) and A549 (FIGS. 20R-20T) cells with rMERS-CoV/TKKA or rMERS-CoV/TKKD at 1 MOI and treated with z-VEID-fmk. MERS-CoV N gene copies in the supernatant samples at the indicated time points were quantified with RT-qPCR (n=3). FIGS. 20U-20V are bar graphs of MERS-CoV-N gene expression in mouse lungs as quantified with RT-qPCR (n=6; FIG. 20U) and infectious titer was determined with plaque assays (n=6; FIG. 20V). hDPP4 KI mice were intranasally inoculated with 3.5×10⁴ PFU rMERS-CoV/TKKA or rMERS-CoV/TKKD. Mouse lung samples were harvested at day 2 and day 4 post infection. Data represented mean and standard deviations from the indicated number of biological repeats.

FIGS. 21A-21C are graphical representations of the suggested model of how caspase-6 facilitates coronavirus replication using MERS-CoV as an example. FIG. 21A illustrates that upon coronavirus infection, the host initiates apoptosis to eliminate infected cells, aiming to terminate virus propagation. The triggered apoptosis cascade leads to activation of executor caspases (caspase-3, -6, -7). FIG. 21B illustrates that MERS-CoV exploits caspase-6 to cleave its N protein, generating N fragments that bind to IRF3, attenuating the activation of IFN signaling, thus benefits virus replication. FIG. 21C illustrates that in the presence of caspase-6 inhibition, N is not cleaved and IFN signaling is more intact, resulting in restricted virus replication.

### DETAILED DESCRIPTION OF THE INVENTION

### I. Definitions

Caspases, also referred to as “cysteine-aspartic proteases”, “cysteine aspartases” or “cysteine-dependent aspartate-directed proteases” refers to a family of protease enzymes playing essential roles in programmed cell death. They are named “caspases” due to their specific cysteine protease activity—a cysteine in its active site nucleophilically attacks and cleaves a target protein only after an aspartic acid residue. The terms “caspase inhibitor” or “inhibitor of caspase signaling” refer to compounds, molecules, and/or compositions that inhibit or reduce the activity of caspases.

“Apoptosis” refers to a type of programmed cell death which causes a cell to stop growing and dividing, and results in a cell's contents flowing into its surrounding environment. Apoptosis is characterized by membrane blebbing, nuclear chromatin condensation, cell shrinkage, DNA breakdown into nucleosome units, and the formation of apoptotic bodies (Park et al., Biomed Res Int., 2021: 3420168; D′Arcy, Cell Biology International, 2019; 43(6):582-592).

“Necroptosis” refers to a type of controlled cell death that has characteristics of both apoptosis and necrosis (Dhuriya and Sharma, Journal of Neuroinflammation, 2018; 15(1):p. 199; Park et al., Biomed Res Int., 2021: 3420168). Necroptosis is characterized by morphology of necrosis, including but not limited to swelling of organelles, increased cell volume, cellular collapse, permeabilization of the plasma membrane, and release of cellular contents, and is dependent on RIPK3 and mixed lineage kinase domain-like proteins (MLKL).

The terms “single guide RNA” or “sgRNA” refer to the polynucleotide sequence comprising the guide sequence, tracr sequence and the tracr mate sequence. “Guide sequence” refers to the around 20 base pair (bp) sequence within the guide RNA that specifies the target site and may be used interchangeably with the terms “guide” or “spacer.”

The terms “individual”, “host”, “subject”, and “patient” are used interchangeably, and refer to a mammal, including, but not limited to, murines, simians, humans, mammalian farm animals and livestock, mammalian sport animals, and mammalian pets.

The term “effective amount” or “therapeutically effective amount” refers to the amount which is able to treat one or more symptoms of a coronavirus infection, reverse the progression of one or more symptoms of a coronavirus infection, halt the progression of one or more symptoms of a coronavirus infection or prevent the occurrence of one or more symptoms of a coronavirus infection in a subject to whom the formulation is administered, for example, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

The term “dosage regime” refers to drug administration regarding formulation, route of administration, drug dose, dosing interval and treatment duration.

The term “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be acceptable” in the sense of being compatible with the other ingredients of a subject composition and not injurious to the patient.

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including lipocalin-2 antagonists may inhibit or reduce the activity and/or quantity of one or more coronaviruses or variants thereof by about 10%, 20%, 30%, 40%, 50%, 75%, 85%, 90%, 95%, or 99% from the activity and/or quantity of the same lipocalin-2 protein or variants thereof in subjects that did not receive or were not treated with the compositions. In some forms, the inhibition and reduction are compared according to the level of mRNAs, proteins, cells, tissues, and organs.

The terms “treating” or “preventing” mean to ameliorate, reduce or otherwise stop a disease, disorder or condition from occurring or progressing in an subject which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with a coronavirus infection and/or a coronavirus-associated disease are mitigated or eliminated, including, but are not limited to, reducing and/or inhibiting rate of replication of the coronavirus in the subject, delaying the progression of the coronavirus infection or coronavirus-associated disease, and/or prolonging survival of individuals.

A weight percent of a component, unless specifically stated to the contrary, is based on the total weight of the formulation or composition in which the component is included.

The terms “protein” or “polypeptide” or “peptide” refer to any chain of more than two natural or unnatural amino acids, regardless of post-translational modification (e.g., glycosylation or phosphorylation), constituting all or part of a naturally occurring or non-naturally occurring polypeptide or peptide.

The term “polynucleotide” or “nucleic acid” or “nucleic acid sequence” refers to a natural or synthetic molecule including two or more nucleotides linked by a phosphate group at the 3′ position of one nucleotide to the 5′ end of another nucleotide. The polynucleotide is not limited by length, and thus the polynucleotide can include deoxyribonucleic acid (DNA) or ribonucleic acid (RNA).

As used herein, the term “activity” refers to a biological activity.

As used herein, the term “pharmacological activity” refers to the inherent physical properties of a Caspase inhibitor. These properties include but are not limited to half-life, solubility, and stability and other pharmacokinetic properties.

Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approx. +/−10%; in other forms the values may range in value either above or below the stated value in a range of approximately +/−5%; in other forms the values may range in value either above or below the stated value in a range of approx. +/−2%; in other forms the values may range in value either above or below the stated value in a range of approx. +/−1%.

### II. Compositions

It has been established that administration of a caspase-6 inhibitor to a subject with a coronavirus infection reduces the replication of pathogenic coronaviruses in the subject. It has further been established that administration of a caspase-6 inhibitor to a subject with a coronavirus infection, ameliorates lung pathology and increases survival in the subject. Typically, the caspase-6 inhibitor is z-VEID-FMK as described below.

A. Caspase-6 Inhibitors

Caspase-6 is a member of the cysteine-aspartic acid protease (caspase) family. Sequential activation of caspases plays a central role in the execution-phase of cell apoptosis. Caspases exist as inactive proenzymes which undergo proteolytic processing at conserved aspartic residues to produce two subunits, large and small, that dimerize to form the active enzyme. This protein is processed by caspases 7, 8 and 10, and is thought to function as a downstream enzyme in the caspase activation cascade.

Caspase-6 inhibitors are described in the literature (See for example, WO 2014/060392, U.S. Pat. No. 9,245,290, Lee, H. et al. (2018) Expert Opinion on Therapeutic Patents Vol. 28(1), pages 47-59; Tubeleviciute-Aydin, A. et al. (2019) Scientific Reports Vol. 9, 5504 (2019), doi:10.1038/s41598-019-41930-7). They include synthetic compounds such as, but not limited to the synthetic peptides commercialized by Merck as Z-Val-Glu(OMe)-Ile-Asp(OMe)-CH2F, also known as Z-VEID-FMK, and Ac-Ala-Ala-Val-Ala-Leu-Leu-Pro-Ala-Val-Leu-Leu-Ala-Leu-Leu-Ala-Pro-Val-Glu-Ile-Asp-CHO (SEQ ID NO:56). These inhibitors are derivatives of peptides that serve as substrates. (Gregoli and Bondurant, 1999, J Cell Physiol, 178, 133-43). The derivatives benzyloxycarbonyl (z-) fluoromethyl-ketone (FMK or CH2F) and difluorophenoxyl (OPH) serve as stabilizing functions. In some forms, the synthetic caspase-6 inhibitors are reversible inhibitors (which is usually the case of aldehyde inhibitors of VEID peptides) or irreversible inhibitors (most conjugates of VEID with chloromethyl, fluoromethyl or acyloxymethyl groups).

In some forms, caspase-6 inhibitor contained in the pharmaceutical composition is a nucleic acid molecule selected from the group comprising a single stranded antisense nucleic acid (ssRNA), a small interfering NA (siRNA), a short hairpin RNA (shRNA), and a microRNA (miRNA).

In some forms, the caspase-6 inhibitor is a small interfering RNAs which lower the expression level of the caspase-6 gene. A short interfering RNA (siRNA) is a small, double-stranded complex, which triggers the RNAi pathway (Bajan and Hutvagner, Cells, 9(1):137 (2020). The nucleotide sequence for the human caspase 6 gene (NCBI Accession No: NG_029187.2) is represented by SEQ ID NO:57. In some forms, the siRNA reduces the expression of the caspase 6-gene by about 5%, about 10%, about 25%, about 50%, about 75%, about 80%, about 85%, about 90%, about 95%, about 99%, or about 100%. siRNA sequences that target caspase-6 are known in the art and are commercially available. For example, the siRNA sequences used in the non-limiting examples were obtained from Dharmacon™ (Catalog #L-004406-00-0005). Exemplary siRNA sequences that can be used to reduce the expression of the caspase-6 gene include CAACAUAACUGAGGUGGAU (SEQ ID NO:62); CAUGGUACAUUCAAGAUUU (SEQ ID NO:63); AAAUAUGGCUCCUCCUUAG (SEQ ID NO:64); and GUUCAAAGGAGACAAGUGU (SEQ ID NO:65). In some forms, the caspase-6 inhibitor can be a (short hairpin) shRNA plasmid or shRNA lentiviral particle, which decreases the expression of the caspase-6 gene.

In some forms, the inhibitor of caspase-6 may be an antisense compound such as an antisense oligonucleotide. Antisense oligonucleotides are single-stranded, highly-modified, synthetic RNA (or DNA) sequences, designed to selectively bind via complementary base-pairing to RNA which encodes the gene of interest (Bajan and Hutvagner, Cells, 9(1):137 (2020). Antisense compounds which inhibit the expression of caspase-6 are described for example in WO 2002/029066A1 by Brown-Driver et al., which is incorporated herein in its entirety.

In some forms, the inhibitor of caspase-6 may be a dominant negative caspase-6 protein, i.e., a mutated caspase-6 molecule which competes with the endogenous caspase-6. In some forms, the caspase-6 inhibitor can also be a vector comprising a nucleotide sequence encoding the above-mentioned siRNA or dominant negative caspase-6. For a given siRNA or a given dominant negative protein, those skilled in the art will be able to identify which nucleotide sequence(s) encode(s) such a siRNA or dominant negative protein, on the basis of the genetic code, the degeneracy of said code, and codon adaptation according to species.

Existing data suggests that coronaviruses activate necroptosis in addition to apoptosis. Necroptosis is a more inflammatory form of cell death when compared to apoptosis. Existing research suggests that inhibition of caspase-8 also inhibits necroptosis (Li et al., Signal Transduct Target Ther., 5(1):235 (2020); thus, using a pan-caspase inhibitor will likely exacerbate necroptosis in a coronavirus infected host, and increase the severity of the coronavirus infection. Selective caspase-6 inhibition does not affect signaling of caspase-8; thereby, having little to no effect on necroptosis. Thus, selective inhibition of caspase-6 signaling is likely more effective at reducing the severity of a coronavirus infection. Thus, in preferred forms, the caspase-6 inhibitor is not a pan-caspase inhibitor. For example, the caspase-6 inhibitor does not inhibit caspase-8 signaling. In other preferred forms, the caspase-6 inhibitor does not increase necroptosis in a cell. In preferred forms, the caspase-6 inhibitor increases apoptosis in infected cells.

1. Z-VEID-FMK

In preferred forms, the caspase-6 inhibitor is Z-VEID-FMK, also known as Z-VEID-fluoromethyl ketone. VEID is the specific recognition sequence for caspase-6/Mch2. The inhibitor Z-VEID-FMK is designed as a methyl ester to facilitate cell permeability. Z-VEID-FMK is commercially available and may be obtained from for example, BPS Bioscience (Catalog #27669-1), R&D Systems (Catalog #FMK006), Abcam (Catalog #AB142025), and APExBIO (Catalog #A1923).

Z-VEID-FMK has the peptide sequence, Z-Val-E(OMe)-Ile-Asp(OMe)-FMK, the molecular formula, C₃₁H₄₅FN₄O₁₀, and the chemical structure as shown in Formula I below.

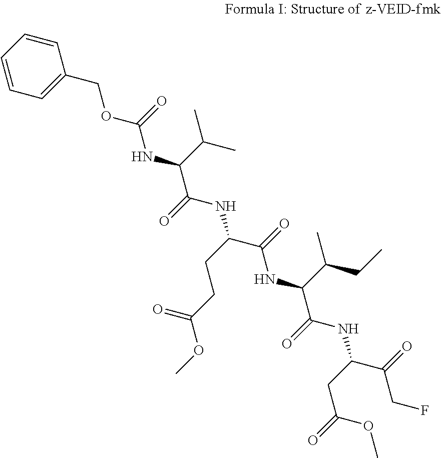 [SMILES: CC[C@H](C)[C@H](NC(=O)[C@H](CCC(=O)OC)NC(=O)[C@@H](NC(=O)OCc1ccccc1)C(C)C)C(=O)N[C@@H](CC(=O)OC)C(=O)CF]

2. Ac-VEID-CHO

In preferred forms, the caspase-6 inhibitor is Ac-VEID-CHO, also known as acetyl-Val-Ile-Asp-aldehyde and VEID-CHO. Ac-VEID-CHO is specific inhibitor of caspase-6.

Ac-VEID-CHO is commercially available and may be obtained from for example, Ambinter (Catalog #AMB19930172), VWR (Catalog #89146-732), and Sigma-Aldrich (Catalog #A6339).

Ac-VEID-CHO has the peptide sequence, NAc-Val-Glu-Ile-Asp-al, the molecular formula, C₂₂H₃₆N₄O₉, and the chemical structure as shown in Formula V below.

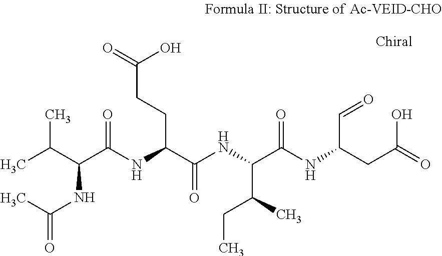 [SMILES: CC[C@H](C)[C@H](NC(=O)[C@H](CCC(=O)O)NC(=O)[C@@H](NC(C)=O)C(C)C)C(=O)N[C@H](C=O)CC(=O)O]

3. Caspase-6 Inhibitory Proteins

In some forms, the caspase-6 inhibitor included in the pharmaceutical composition is an inhibitory caspase-6 protein. Typically, the inhibitory caspase-6 protein reduces the expression and/or function of the caspase-6 protein. In preferred forms, the inhibitory caspase-6 protein reduces the expression and/or function of the Human Caspase-6 Protein. Human Caspase-6 Proteins are represented by SEQ ID NO:58, SEQ ID NO:59, SEQ ID NO:60, and SEQ ID NO:61.

Human Casp6 (Uniprot ID P55212 Alpha, 293
amino acids):
(SEQ ID NO: 58)
MSSASGLRRGHPAGGEENMTETDAFYKREMFDPAEKYKMDHRRRGIALIF
NHERFFWHLTLPERRGTCADRDNLTRRFSDLGFEVKCFNDLKAEELLLKI
HEVSTVSHADADCFVCVFLSHGEGNHIYAYDAKIEIQTLTGLFKGDKCHS
LVGKPKIFIIQACRGNQHDVPVIPLDVVDNQTEKLDTNITEVDAASVYTL
PAGADFLMCYSVAEGYYSHRETVNGSWYIQDLCEMLGKYGSSLEFTELLT
LVNRKVSQRRVDFCKDPSAIGKKQVPCFASMLTKKLHFFPKSN
Human Casp6 (Uniprot ID P55212 Beta, 204
amino acids):
(SEQ ID NO: 59)
MSSASGLRRGHPAVSTVSHADADCFVCVFLSHGEGNHIYAYDAKIEIQTL
TGLFKGDKCHSLVGKPKIFIIQACRGNQHDVPVIPLDVVDNQTEKLDTNI
TEVDAASVYTLPAGADFLMCYSVAEGYYSHRETVNGSWYIQDLCEMLGKY
GSSLEFTELLTLVNRKVSQRRVDFCKDPSAIGKKQVPCFASMLTKKLHFF
PKSN
Human Casp6 (Uniprot ID D6RHU3, 143
amino acids):
(SEQ ID NO: 60)
MTETDAFYKREMFDPAEKYKMDHRRRGIALIFNHERFFWHLTLPERRGTC
ADRDNLTRRFSDLGFEVKCFNDLKAEELLLKIHEVSTVSHADADCFVCVF
LSHGEGNHIYAYDAKIEIQTLTGLFKGDKCHSLVGKPKIFIIQ
Human Casp6 (Uniprot ID D6RBM3; 80
amino acids):
(SEQ ID NO: 61)
MSSASGLRRGHPAGGEENMTETDAFYKREMFDPAEKYKMDHRRRGIALIF
NHERFFWHLTLPERRGTCADRDNLTRSVNC

In some forms, the inhibitory caspase-6 protein is a synthetic peptide. Synthetic peptides are known and commercially available e.g., Human Caspase 6 Synthetic Peptide blocker (ThermoFisher Scientific, Catalog #PEP-0235 and Catalog #PEP-0234).

In some forms, the caspase-6 inhibitor is a caspase-6 inhibitor antibody. Exemplary antibodies that can be used include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each include four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

In some forms, the caspase-6 protein inhibitors are variants and fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the non-modified antibody or antibody fragment. Exemplary fragments and fusions include, but are not limited to, single chain antibodies, single chain variable fragments (scFv), di-scFv, tri-scFv, diabody, triabody, tetrabody, disulfide-linked Fvs (sdFv), Fab′, F(ab′)2, Fv, and single domain antibody fragments (sdAb).

Caspase-6 antibodies are known and commercially available. In some forms, the caspase-6 antibody is an unconjugated antibody. Exemplary commercially available unconjugated caspase-6 antibodies include but are not limited to:
(i) Anti-Caspase-6/CASP-6 monoclonal antibody (ABCAM Cat #: ab108335) (Zhu Y et al., Naunyn Schmiedebergs Arch Pharmacol 394:809-817 (2021); Wang Y et al. Maxim. Front Pharmacol 11:572387 (2020); Mou Z et al. Front Genet 11:566918 (2020); Volpin V et al. Cancer Immunol Res 8:1163-1179 (2020); Chen Y J et al. Antioxid Redox Signal 31:109-126 (2019); Ma J et al. Cell Res 26:713-27 (2016)).
(ii) Anti-Caspase-6/CASP-6 antibody [EPR18043] (ABCAM Cat #: ab185645) (Chen Y et al., Cell Death Discov 7:290 (2021); Wu W et al., mBio 12:e0100521 (2021).
(iii) Caspase 6 Recombinant Rabbit Monoclonal Antibody (SC56-09) by Invitrogen (Catalog #MA5-32201).
(iv) Caspase 6/p18/p11 Polyclonal Antibody (ThermoFisher Scientific; Catalog #10198-1-AP); (Zhang et al., Biotechnology and Bioengineering, 114(11):2539-2549 (2017); Li T, et al., Biomark Med., 10(12):1251-1260. (2016); Liao et al., Chem Biol Interact., 242:91-8 (2015)).

In some forms, the caspase-6 antibody is a conjugated caspase-6 antibody. In some forms, the caspase-6 antibody has been conjugated to a substrate such as a toxin, enzyme, drug, or inorganic molecule. In some forms, the conjugated antibody is an immune-stimulating caspase-6 antibody conjugate. Immune-stimulating antibody conjugates (ISACs) are engineered monoclonal antibodies coupled to synthetic PRR ligands that potentiate immune responses by triggering the production of pro-inflammatory cytokines and chemokines.

4. Small Molecule Caspase-6 Inhibitors

Small molecule drugs are compounds manufactured through chemical synthesis and have well-defined chemical structures. These drugs are typically either synthesized from or meant to replicate natural compounds produced by plants, fungi, and bacteria (Gurevich and Gurevich, Handb Exp Pharmacol., 219:1-12 (2014)). Small molecule drugs are effective allosteric modifiers and enzyme inhibitors, and they are used to target extracellular proteins or intracellular receptors in the cytosol, and nuclei, among other cellular components (Gurevich and Gurevich, Handb Exp Pharmacol., 219:1-12 (2014)). Small molecules offer several advantages as antiviral agents, including ease of synthesis, oral bioavailability, and the potential for targeting specific host factors.

Thus, in some forms, the small molecule drugs inhibit caspase-6 by a variety of ways including but not limited to disrupting viral replication, reducing inflammatory responses, and enhancing host antiviral defenses. Exemplary small molecule inhibitors of caspase-6 expression include but are not limited to the compounds S10G and C13 as described in Tubeleviciute-Aydin, et al. Identification of Allosteric Inhibitors against Active Caspase-6. Sci Rep 9, 5504 (2019) which is incorporated herein in its entirety. The structure of S10G and C13 are shown below.

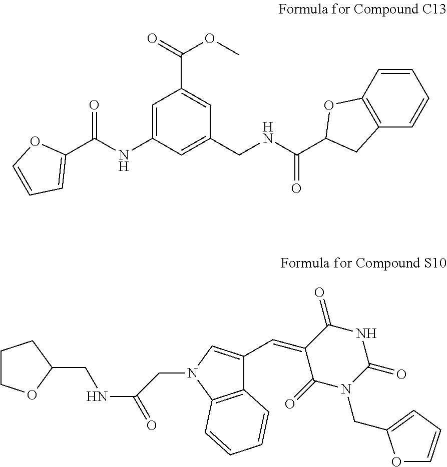 [SMILES: COC(=O)c1cc(CNC(=O)C2Cc3ccccc3O2)cc(NC(=O)c2ccco2)c1 ; O=C(Cn1cc(/C=C2/C(=O)NC(=O)N(Cc3ccco3)C2=O)c2ccccc21)NCC1CCCO1]

B. Formulations

Formulations of, and pharmaceutical compositions including one or more caspase-6 inhibitors are provided. The formulations and compositions can include the caspase-6 inhibitor (s) and a pharmaceutically acceptable carrier together in the same admixture, or in separate formulations. Therefore, pharmaceutical compositions including one or more caspase-6 inhibitors are described.

1. Delivery Vehicles

In some forms, the one or more caspase-6 inhibitor (s), and optionally additional therapeutic, prophylactic, and/or diagnostic agents are administered and taken up into the cells of a subject with the aid of a delivery vehicle. Appropriate delivery vehicles for the disclosed compositions are known in the art and can be selected to suit the particular formulation. For example, in some forms, the composition is incorporated into or encapsulated by a nanoparticle, microparticle, micelle, synthetic lipoprotein particle, or carbon nanotube. For example, the compositions can be incorporated into a vehicle such as polymeric microparticles, which provide controlled release of the caspase-6 inhibitor (s). In some forms, release of the caspase-6 inhibitor (s) and/or additional therapeutic, prophylactic, and/or diagnostic agents is controlled by diffusion of the active compositions out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

2. Pharmaceutical Compositions

Pharmaceutical compositions containing a therapeutically effective amount of one or more caspase-6 inhibitor (s), and optionally additional therapeutic, prophylactic, and/or diagnostic agents with or without a delivery vehicle are provided. Pharmaceutical compositions can be for administration by parenteral (intramuscular, intraperitoneal, intravenous (IV) or subcutaneous injection), enteral, or transmucosal (nasal, vaginal, rectal, or sublingual) routes of administration or using bioerodible inserts and can be formulated in dosage forms appropriate for each route of administration.

a. Formulations for Parenteral Administration

Caspase-6 inhibitors and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacterium retaining filter, by incorporating sterilizing agents into the compositions, by irradiating the compositions, or by heating the compositions.

These particular aqueous solutions are especially suitable for intranasal or intratracheal administration to the primary infection site, i.e., the respiratory tract. These particular aqueous solutions are also suitable for intravenous, intramuscular, subcutaneous, and intraperitoneal administration. In this connection, sterile aqueous media which can be employed will be known to those of skill in the art in light of the present disclosure. For example, one dosage could be dissolved in 1 ml of isotonic NaCl solution and either added to variation in dosage will necessarily occur depending on the condition of the subject being treated. The person responsible for administration will, in any event, determine the appropriate dose for the individual subject.

b. Dry Formulations

In some forms, compositions of, pharmaceutical compositions of one or more caspase-6 inhibitor (s), and optionally additional therapeutic, prophylactic, and/or diagnostic agents are dried or lyophilized. Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial.

The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

c. Additional Therapeutic, Prophylactic or Diagnostic Agents

In some forms, the pharmaceutical compositions include one or more additional active agents. Therefore, in some forms, the pharmaceutical composition includes one or more caspase-6 inhibitor (s), in addition to one, two, three, or more therapeutic, prophylactic, and/or diagnostic agents. The additional agents can be included together with the one or more caspase-6 inhibitor (s) or may be a separate composition.

In some forms, the additional therapy is a conventional vaccine or treatment for an infectious disease, more preferably a conventional vaccine or treatment for a coronavirus. For example, in some forms, the additional therapy or vaccine is a treatment for or a vaccine against SARS-COV-2. In some forms, the additional treatment is a prophylactic drug against a coronavirus infection.

In other forms, the compositions include one or more additional molecules that enhance or induce an anti-inflammatory response within the recipient. Exemplary molecules include cytokines and co-stimulatory molecules. For example, in some forms, the composition administered to the subject further includes of a co-stimulatory molecule, a growth factor, or a cytokine. Exemplary molecules include but are not limited to IFN-signaling molecules for example IFIT1, IFIT2, IFIT3, IFITM3, TRIM22, and/or OAS1. In some forms, the one or more additional molecules that enhance or induce an anti-inflammatory response within the recipient reduces the expression of one or more pro-inflammatory cytokines and/or chemokines such as but not limited to IL6, IL1β, TNFα, IFNγ, IP10, IP6, MCP1, CXCL3, CXCL5, and/or CXCL9.

In some forms, the one or more additional molecules is administered to the subject before, at the same time, and after the pharmaceutical composition containing the one or more caspase-6 inhibitor (s) is administered. In some forms, the one or more additional molecules are administered to the subject at the same or different site that the pharmaceutical composition includes one or more caspase-6 inhibitor (s), via the same or a different route.

d. Excipients

The compositions may be formulated with one or more excipients and/or carriers appropriate to the indicated route of administration. In some forms, the one or more caspase-6 inhibitor (s) are formulated in a manner amenable for the treatment of human and/or veterinary patients. In some forms, the pharmaceutical compositions include admixing or combining one or more of the caspase-6 inhibitor (s) with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some forms, e.g., for oral administration, the pharmaceutical compositions may be tableted or encapsulated. In some forms, the one or more caspase-6 inhibitor (s) may be slurried in water, polyethylene glycol, propylene glycol, ethanol, corn oil, cottonseed oil, peanut oil, sesame oil, benzyl alcohol, sodium chloride, and/or various buffers. In some forms, the pharmaceutical compositions may be subjected to pharmaceutical operations, such as sterilization, and/or may contain carriers and/or excipients such as preservatives, stabilizers, wetting agents, emulsifiers, encapsulating agents such as lipids, dendrimers, polymers, proteins such as albumin, nucleic acids, and buffers.

e. Dosage Units

In some forms, it may be advantageous to formulate compositions in dosage unit form for ease of administration and uniformity of dosage. “Dosage unit form” as used herein refers to physically discrete units suited as unitary dosages for the patients to be treated; each unit containing a predetermined quantity of therapeutic agent calculated to produce the desired therapeutic effect in association with the required pharmaceutical carrier. In some forms, the specification for the dosage unit forms of the invention is dictated by and directly dependent on (a) the unique characteristics of the therapeutic agent and the particular therapeutic effect to be achieved, and (b) the limitations inherent in the art of compounding such a therapeutic agent for the treatment of a selected condition in a patient. In some forms, the one or more caspase-6 inhibitor (s) are administered at a therapeutically effective dosage sufficient to treat a condition associated with a condition in a patient. For example, the efficacy of a pharmaceutical composition containing one or more caspase-6 inhibitor (s) can be evaluated in an animal model system that may be predictive of efficacy in treating the disease in a human or another animal.

In some forms, the effective dose range for the therapeutic agent can be extrapolated from effective doses determined in animal studies for a variety of different animals. Precise amounts of the pharmaceutical composition depend on the judgment of the practitioner and are specific to each individual. Other factors affecting the dose include the physical and clinical state of the patient, the route of administration, the intended goal of treatment and the potency, stability, and toxicity of the particular pharmaceutical composition.

The actual dosage amount of a bacterial composition of the present disclosure administered to a patient may be determined by physical and physiological factors such as type of animal treated, age, sex, body weight, severity of condition, the type of condition being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some forms, the agent is administered once a day.

The composition may be administered on a routine schedule. As used herein, a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other forms, the invention provides that the agent(s) may be taken orally and that the timing of which is or is not dependent upon food intake. Thus, for example, the agent can be taken every morning and/or every evening, regardless of when the patient has eaten or will eat.

### III. Methods of Use

It has been established that caspase-6 inhibitor (s) can be administered to a subject with a coronavirus infection to reduce the replication of pathogenic coronaviruses in the subject. It has further been established caspase-6 inhibitor (s) can be administered to a subject with a coronavirus infection to ameliorate lung pathology and increase survival in the subject. Therefore, methods of treating a subject with a coronavirus infection with a pharmaceutical composition comprising a caspase-6 inhibitor are provided. Preferably, the caspase-6 inhibitor comprised in the pharmaceutical composition is z-VEID-FMK or Ac-VEID CHO as described above.

A. Methods of Treatment

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “inhibit” means to reduce or decrease in activity or expression. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the disclosed compounds.

1. Downregulation of Inflammatory Responses

In preferred forms, the pharmaceutical composition containing the caspase-6 inhibitor is administered to a subject in need thereof, in an amount effective to reduce the production, inhibit the activation, or inhibit a signaling pathway of caspase-6. In some forms, the pharmaceutical composition containing the caspase-6 inhibitor is administered to a subject in need thereof, in an amount effective to reduce the expression of one or more pro-inflammatory cytokines and/or chemokines including but not limited to IL6, IL1β, TNFα, IFNγ, IP10, IP6, MCP1, CXCL3, CXCL5, and/or CXCL9. In some forms, the pharmaceutical composition containing the caspase-6 inhibitor is administered to a subject in need thereof, in an amount effective to upregulate the expression of one or more IFN-signaling genes including but not limited to IFIT1, IFIT2, IFIT3, IFITM3, TRIM22, and/or OAS1 following treatment.

2. Reduction of Coronavirus-Induced Symptoms

In preferred forms, the pharmaceutical composition containing the caspase-6 inhibitor is administered to a subject in need thereof, in an amount effective to reduce coronavirus replication in small airways and alveoli of the lungs. In some forms, the pharmaceutical composition containing the caspase-6 inhibitor is administered to the subject in need thereof in an amount effective to reduce bronchiolitis, alveolitis, and vasculitis. In some forms, the pharmaceutical composition is administered to the subject in need thereof, in an amount effective to reduce bronchiolar epithelial cell death and desquamation, alveolar space mononuclear cell infiltration, protein rich fluid exudation, alveolar hemorrhage, damage to alveolar structure, pulmonary blood vessel wall inflammation and endothelium infiltration, focal alveolar septal congestion and perivascular infiltration, and lower alveolar space immune cells. In some forms, the pharmaceutical composition is administered to a subject in need thereof, in an amount effective to reduce body weight loss and improve survival.

B. Conditions to Be Treated

In some forms, the pharmaceutical composition containing the one or more caspase-6 inhibitor (s) can be used to treat a subject with a coronavirus infection or a subject at risk of developing one or more symptoms associated with a coronavirus infection. In some forms, the pharmaceutical composition containing the one or more caspase-6 inhibitor (s) can be used to treat a subject having an elevated risk of developing one or more symptoms associated with a severe coronavirus infection, such as systemic inflammatory response syndrome, sepsis, or septic shock. In some forms, the methods of treatment with the pharmaceutical compositions containing the one or more caspase-6 inhibitor (s) are based on determining that the subject has one or more viral markers that are known in art to increase risk for the subject to develop moderate to severe infection with a coronavirus.

1. Coronaviruses and SARS-CoV-2

The coronaviruses (order Nidovirales, family Coronaviridae, and genus Coronavirus) are a diverse group of large, enveloped, positive-stranded RNA viruses that cause respiratory and enteric diseases in humans and other animals.

Coronaviruses typically have narrow host specificity and can cause severe disease in many animals, and several viruses, including infectious bronchitis virus, feline infectious peritonitis virus, and transmissible gastroenteritis virus, are significant veterinary pathogens. Human coronaviruses (HCoVs) are found in both group 1 (HCoV-229E) and group 2 (HCoV-OC43) and are historically responsible for ˜30% of mild upper respiratory tract illnesses.

At ˜30,000 nucleotides, their genome is the largest found in any of the RNA viruses. There are three groups of coronaviruses; groups 1 and 2 contain mammalian viruses, while group 3 contains only avian viruses. Within each group, coronaviruses are classified into distinct species by host range, antigenic relationships, and genomic organization. The genomic organization is typical of coronaviruses, with the characteristic gene order (5′-replicase [rep], spike [S], envelope [E], membrane [M], nucleocapsid [N]-3′) and short untranslated regions at both termini. The SARS-CoV rep gene, which includes approximately two-thirds of the genome, encodes two polyproteins (encoded by ORFla and ORF1b) that undergo co-translational proteolytic processing. There are four open reading frames (ORFs) downstream of rep that are predicted to encode the structural proteins, S, E, M, and N, which are common to all known coronaviruses.

In some forms, the coronavirus disease to be treated is COVID associated with SARS-CoV-2 betacoronavirus of the subgenus Sarbecovirus. SARS-CoV-2 viruses share approximately 79% genome sequence identity with the SARS-CoV virus identified in 2003. The genome organization of SARS-CoV-2 viruses is shared with other betacoronaviruses; six functional open reading frames (ORFs) are arranged in order from 5′ to 3′: replicase (ORF1a/ORF1b), spike (S), envelope (E), membrane (M) and nucleocapsid (N). In addition, seven putative ORFs encoding accessory proteins are interspersed between the structural genes.

In some forms, the coronavirus is a variant of SARS-CoV-2, such as SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, or SARS-CoV-2 B.1.1.529 (Omicron variant). In some forms, the coronavirus can be a sub-variant of the SARS-CoV-2 B.1.1.7 (Alpha variant), a sub-variant of the SARS-CoV-2 B.1.351 (Beta variant), a sub-variant of the SARS-CoV-2 P.1 (Gamma variant), a sub-variant of the SARS-CoV-2 B.1.617, a sub-variant of the SARS-CoV-2 B.1.617.1 (Kappa variant), a sub-variant of the SARS-CoV-2 B.1.621 (Mu variant), a sub-variant of the SARS-CoV-2 B.1.617.2 (Delta variant), a sub-variant of the SARS-CoV-2 B.1.617.3, or a sub-variant of the SARS-CoV-2 B.1.1.529 (Omicron variant), or a sub-variant derived from a descendent lineage of one or more of the foregoing sub-variants. For example, when the SARS-CoV-2 variant is an Omicron variant, the Omicron sub-variant can be a BA.1 sub-variant, a BA.2 sub-variant, a BA.3 sub-variant, a BA.4 sub-variant, a BA.5 sub-variant, or a BA.1/BA.2 circulating recombinant sub-variant such as XE.

In some forms, when the pathogenic coronavirus is SARS-CoV-2, the SARS-CoV-2 variant can be a variant of the wild-type strain of the coronavirus. “Wild-type” as used herein, refers to the original strain of coronavirus considered to be the background strain of the coronavirus containing no major mutations.

i. Symptoms of COVID-19

Patients with SARS-CoV-2 infection can experience a range of clinical manifestations, from no symptoms to critical illness. In general, adults with SARS-CoV-2 infection can be grouped into the following severity of illness categories; however, the criteria for each category may overlap or vary across clinical guidelines and clinical trials, and a patient's clinical status may change over time.
(i) Asymptomatic or pre-symptomatic infection: individuals who test positive for SARS-CoV-2 using a virologic test (i.e., a nucleic acid amplification test or an antigen test) but who have no symptoms that are consistent with COVID-19.
(ii) Mild illness: individuals who have any of the various signs and symptoms of COVID-19 (e.g., fever, cough, sore throat, malaise, headache, muscle pain, nausea, vomiting, diarrhea, loss of taste and smell) but who do not have shortness of breath, dyspnea, or abnormal chest imaging.
(iii) Moderate Illness: Individuals who show evidence of lower respiratory disease during clinical assessment or imaging and who have an oxygen saturation (SpO2) ≥94% on room air at sea level.
(iv) Severe illness: individuals who have SpO2<94% on room air at sea level, a ratio of arterial partial pressure of oxygen to fraction of inspired oxygen (PaO2/FiO2) <300 mm Hg, a respiratory rate >30 breaths/min, or lung infiltrates >50%. These patients may experience rapid clinical deterioration. Oxygen therapy should be administered immediately using a nasal cannula or a high-flow oxygen device. If secondary bacterial pneumonia or sepsis is suspected, administer empiric antibiotics, re-evaluate the patient daily, and de-escalate or stop antibiotics if there is no evidence of bacterial infection.
(v) Critical illness: individuals who have acute respiratory distress syndrome, septic shock that may represent virus-induced distributive shock, cardiac dysfunction, an exaggerated inflammatory response, and/or exacerbation of underlying comorbidities. In addition to pulmonary disease, patients with critical illness may also experience cardiac, hepatic, renal, central nervous system, or thrombotic disease.

Patients with certain underlying comorbidities are at a higher risk of progressing to severe COVID-19. These comorbidities include being aged ≥65 years; having cardiovascular disease, chronic lung disease, sickle cell disease, diabetes, cancer, obesity, or chronic kidney disease; being pregnant; being a cigarette smoker; being a transplant recipient; and receiving immunosuppressive therapy.

In some cases, patients with COVID-19 may have additional infections that are noted when they present for care or that develop during the course of treatment. These coinfections may complicate treatment and recovery. Older patients or those with certain comorbidities or immunocompromising conditions may be at higher risk for these infections.

In some forms, the pharmaceutical composition containing the one or more caspase-6 inhibitor (s) can be used to reduce the replication of SARS-CoV-2 variants that predispose the host to developing severe COVID. In further forms, the pharmaceutical composition containing the one or more caspase-6 inhibitor (s) can be used to treat patients having an elevated risk of developing one or more symptoms associated with severe COVID-19 as a result of SARS-CoV-2 infection. In these cases, the patients carrying these SARS-CoV-2 variants are likely to develop one or more symptoms associated with severe illness, critical illness, and additional complications. The disclosed the pharmaceutical composition containing the one or more caspase-6 inhibitor (s) can be used to treat a subject as at risk of developing severe COVID in order to reduce the infection and replication of the virus at least by 50%, 60%, 70%, 80%, 90%, 95%, 97%, or 99%.

2. SARS-CoV

In some forms, the pharmaceutical composition containing the one or more caspase-6 inhibitor (s) can be used to reduce the replication of SARS-CoV variants that predispose the host to developing severe acute respiratory syndrome, otherwise known as SARS. SARS is caused by the SARS coronavirus, known as SARS CoV. SARS CoV is believed to be a strain of the coronavirus usually only found in small mammals that have mutated, thereby enabling it to infect humans.

A wide range of clinical manifestations are seen in patients with SARS from mild, moderate, to severe and rapidly progressive and fulminant disease. The estimated mean incubation period of SARS-CoV infection was 4.6 days (95% CI, 3.8-5.8 days) and 95% of illness onset occurred within 10 days. The mean time from symptom onset to hospitalization was between 2 and 8 days but was shorter toward the later phase of the epidemic. The mean time from symptom onset to need for invasive mechanical ventilation (IMV) and to death was 11 and 23.7 days, respectively.

The major clinical features of SARS are fever, rigors, chills, myalgia, dry cough, malaise, dyspnea, and headache. Sore throat, sputum production, rhinorrhea, nausea, vomiting, and dizziness are less common. Watery diarrhea was present in 40% to 70% of patients with SARS and tended to occur about 1 week after illness onset. SARS-CoV was detected in the serum and cerebrospinal fluid of 2 patients complicated by status epilepticus. Elderly patients with SARS-CoV infection might present with poor appetite, a decrease in general well-being, fracture as a result of fall, and confusion, but some elderly subjects might not be able to mount a febrile response. In contrast, SARS-CoV infection in children aged less than 12 years was generally mild, whereas infection in teenagers resembled that in adults. There was no mortality among young children and teenagers. SARS-CoV infection acquired during pregnancy carried a case fatality rate of 25% and was associated with a high incidence of spontaneous miscarriage, preterm delivery, and intrauterine growth retardation without perinatal SARS-CoV infection among the newborn infants.

Asymptomatic SARS-CoV infection was uncommon in 2003; a meta-analysis had shown overall sero-prevalence rates of 0.1% (95% CI, 0.02-0.18) for the general population and 0.23% for health care workers (95% CI, 0.02-0.45) in comparison with healthy blood donors, others from the general community, or patients without SARS-CoV infection recruited from the health care setting (0.16%, 95% CI, 0-0.37).

The clinical course of patients with SARS-CoV infection seemed to manifest in different stages. In the first week of illness of SARS-CoV infection, many patients presented with fever, dry cough, myalgia, and malaise that might improve despite the presence of lung consolidation and rising viral loads on serial samples. During the second week, many patients experienced recurrence of fever, worsening consolidation, and respiratory failure, while about 20% of patients progressed to ARDS requiring IMV. Peaking of viral load on day 10 of illness corresponded temporally to peaking of the extent of consolidation radiographically, and a maximal risk of nosocomial transmission, particularly to health care workers.

The disclosed pharmaceutical compositions containing one or more caspase-6 inhibitors may be administered to a patient infected with SARS-CoV and/or variants thereof, in order to reduce the infection and replication of the virus at least by 50%, 60%, 70%, 80%, 90%, 95%, 97%, or 99%, thereby ameliorating the symptoms described above.

3. MERS-CoV

In some forms, the pharmaceutical composition containing the one or more caspase-6 inhibitor (s) can be used to reduce the replication of Middle East respiratory syndrome-related coronavirus (MERS-CoV) variants that predispose the host to developing Middle East Respiratory Syndrome (MERS). MERS-CoV is a coronavirus believed to be originally from bats. However, humans are typically infected from camels, either during direct contact or indirectly. Spread between humans typically requires close contact with an infected person. As of 2021, there is no specific vaccine or treatment for the disease, although attempts are being made.

The virus MERS-CoV is a member of the beta group of coronavirus, Betacoronavirus, lineage C. MERS-CoV genomes are phylogenetically classified into two clades, clade A and B. The earliest cases were of clade A clusters, while the majority of more recent cases are of the genetically distinct clade B. MERS-CoV is closely related to the Tylonycteris bat coronavirus HKU4 and Pipistrellus bat coronavirus HKU5.

The specific exposures that lead to sporadic MERS-CoV infections are unknown, therefore it is challenging to estimate the incubation period in primary cases. However, based on data from cases of human-to-human transmission, the incubation period is a median of 5-7 days, with a range of 2-14 days (median 5·2 days [95% CI 1·9-14·7]). Immunocompromised patients can present with longer incubation periods of up to 20 days.

The clinical presentation of patients infected with MERS-CoV ranges from asymptomatic or mild upper respiratory illness to rapidly progressive pneumonitis, respiratory failure, acute respiratory distress syndrome, septic shock, and multiorgan failure with fatal outcome. Some individuals remain asymptomatic whereas some go on to develop mild disease, which is why WHO classifies these individuals as mild or asymptomatic. Asymptomatic-to-mild infection rates of 25-50% have been reported. The signs and symptoms associated with MERS are non-specific, with or without multisystem involvement, and thus could be mistaken for other causes of respiratory tract or gastrointestinal illnesses. Therefore, the clinical diagnosis of MERS can be easily missed. Patients with MERS can typically present with fever, chills, rigors, headache, a non-productive cough, sore throat, arthralgia, and myalgia followed by dyspnoea. Other associated symptoms include coryza, nausea, vomiting, dizziness, sputum production, diarrhea, and abdominal pain. Some patients with MERS can present with atypical symptoms of mild respiratory illness without a fever and a gastrointestinal illness that precedes the development of pneumonia. Neuromuscular manifestations include hypersomnolence, weakness, and tingling in the extremities similar to Guillain-Barr6 syndrome or virus-related sensory neuropathy.68 Co-infection of MERS-CoV with other respiratory viruses (such as parainfluenza virus, rhinovirus, influenza A or B virus, respiratory syncytial virus, enteroviruses, and human metapneumovirus) and nosocomial bacterial infections has been reported in patients receiving intensive care.

The disclosed pharmaceutical compositions containing one or more caspase-6 inhibitors may be administered to a patient infected with MERS-CoV and/or variants thereof, in order to reduce the infection and replication of the virus at least by 50%, 60%, 70%, 80%, 90%, 95%, 97%, or 99%, thereby ameliorating the symptoms described above.

4. Common Human Coronaviruses

Unlike the highly pathogenic SARS-CoV, MERS-CoV, and 2019-nCoV, the four so-called common HCoVs generally cause mild upper-respiratory tract illness and contribute to 15%-30% of cases of common colds in human adults, although severe and life-threatening lower respiratory tract infections can sometimes occur in infants, elderly people, or immunocompromised patients. In some forms, the pharmaceutical composition containing the one or more caspase-6 inhibitor (s) may be administered to a patient in need thereof to reduce the replication and ameliorate the pathology associated with one or more of the four common HCoVs.

Human coronavirus 229E (HCoV-229E) is a species of coronavirus which infects humans and bats. HCoV-229E is a member of the genus Alphacoronavirus and subgenus Duvinacovirus. It is an enveloped, positive-sense, single-stranded RNA virus which enters its host cell by binding to the APN receptor. HCoV-229E is associated with a range of respiratory symptoms, ranging from the common cold to high-morbidity outcomes such as pneumonia and bronchiolitis. However, such high morbidity outcomes are almost always seen in cases with co-infection with other respiratory pathogens. In some forms, HCoV-229E may cause acute respiratory distress syndrome (ARDS). HCoV-229E is also among the coronaviruses most frequently co-detected with other respiratory viruses, particularly with human respiratory syncytial virus (HRSV). The disclosed pharmaceutical compositions containing one or more caspase-6 inhibitors may be administered to a patient infected with HCoV-229E and/or variants thereof, in order to reduce the infection and replication of the virus at least by 50%, 60%, 70%, 80%, 90%, 95%, 97%, or 99%, thereby ameliorating the described symptoms.

Human coronavirus NL63 (HCoV-NL63) is a species of coronavirus, specifically a Setracovirus from among the Alphacoronavirus genus. The virus is an enveloped, positive-sense, single-stranded RNA virus which enters its host cell by binding to ACE2.

The virus is found primarily in young children, the elderly, and immunocompromised patients with acute respiratory illness. It also has a seasonal association in temperate climates. The evolution of HCoV-NL63 appears to have involved recombination between an ancestral NL63-like virus circulating in African Triaenops afer bats and a CoV 229E-like virus circulating in Hipposideros bats. Recombinant viruses can arise when two viral genomes are present in the same host cell. The first cases of the infection with HCoV-NL63 were found in young children with severe lower respiratory tract infections admitted to hospitals. While the clinical presentation of the virus can be severe, it has also been found in mild cases of respiratory infection. The comorbidity of HCoV-NL63 with other respiratory infections, has made the specific symptoms of the virus difficult to pinpoint. A study of clinical symptoms in HCoV-NL63 patients without secondary infection, reported the most common symptoms to be fever, cough, rhinitis, sore throat, hoarseness, bronchitis, bronchiolitis, pneumonia, and croup. An early study investigating children with lower respiratory tract illness, found that HCoV-NL63 was more commonly found in outpatients than hospitalized patients, suggesting that it is a common cold virus similar to HCoV-229E and HCoV-OC43, which generally cause less severe symptoms. The disclosed pharmaceutical compositions containing one or more caspase-6 inhibitors may be administered to a patient infected with HCoV-NL63 and/or variants thereof, in order to reduce the infection and replication of the virus at least by 50%, 60%, 70%, 80%, 90%, 95%, 97%, or 99%, thereby ameliorating the described symptoms.

Human coronavirus OC43 (HCoV-OC43) is a member of the species Betacoronavirus 1, which infects humans and cattle. The infecting coronavirus is an enveloped, positive-sense, single-stranded RNA virus that enters its host cell by binding to the N-acetyl-9-O-acetylneuraminic acid receptor. Four HCoV-OC43 genotypes (A to D) have been identified, with genotype D most likely arising from genetic recombination. The complete genome sequencing of genotypes C and D and bootscan analysis shows recombination events between genotypes B and C in the generation of genotype D. Of 29 viral variants identified, none belong to the more ancient genotype A. Symptoms of an infection with HCoV-OC43 are as described for HCoV-229E and HCoV-NL63. The disclosed pharmaceutical compositions containing one or more caspase-6 inhibitors may be administered to a patient infected with HCoV-OC43 and/or variants thereof, in order to reduce the infection and replication of the virus at least by 50%, 60%, 70%, 80%, 90%, 95%, 97%, or 99%, thereby ameliorating the above-described symptoms.

Human coronavirus HKU1 (HCoV-HKU1) is an enveloped, positive-sense, single-stranded RNA virus which like the OC43 virus, enters its host cell by binding to the N-acetyl-9-O-acetylneuraminic acid receptor. HCoV-HKU1 has the Hemagglutinin esterase (HE) gene, which distinguishes it as a member of the genus Betacoronavirus and subgenus Embecovirus. Symptoms of an infection with HCoV-HKU1 are as described for HCoV-229E and HCoV-NL63. The disclosed pharmaceutical compositions containing one or more caspase-6 inhibitors may be administered to a patient infected with HCoV-HKU1 and/or variants thereof, in order to reduce the infection and replication of the virus at least by 50%, 60%, 70%, 80%, 90%, 95%, 97%, or 99%, thereby ameliorating the above-described symptoms.

5. Non-Human Coronaviruses

The coronavirus infection to be treated may be caused by an alpha coronavirus or beta coronavirus that can infect a non-human mammal. In some forms, the alpha coronavirus can canine enteric coronavirus (CECoV), feline coronavirus (FCoV), porcine respiratory coronavirus (PRCV), porcine epidemic diarrhea virus (PEDV), or transmissible gastroenteritis virus (TGEV). In some forms, the alpha coronavirus can be a variant derived from rhinolophus bat coronavirus HKU2 (Bat-CoV HKU2) or miniopterus bat coronavirus HKU8 (Bat-CoV HKU8). In some forms, the beta coronavirus can be canine respiratory coronavirus (CRCoV), murine coronavirus (M-CoV), porcine hemagglutinating encephalomyelitis virus (PHEV), hedgehog coronavirus 1, bovine coronavirus (B-CoV), or equine coronavirus (E-CoV). In some forms, the beta coronavirus can be a variant derived from tylonycteris bat coronavirus HKU4 (Bat-CoV HKU4), pipistrellus bat coronavirus HKU5 (Bat-CoV HKU5), or rousettus bat coronavirus HKU9 (Bat-CoV HKU9) The coronavirus infection to be treated may also be caused by a gamma coronavirus or a delta coronavirus. In some forms, the gamma coronavirus can be Avian Infectious Bronchitis (AIBV) or Beluga Whale CoV SW1. In some forms, the delta coronavirus can be Bulbul CoV HKU11 (BuCoV HKU11), Thrush CoV HKU12 (ThCoV HKU12), Munia CoV HKU13 (MunCoV HKU13), Porcine CoV HKU15 (PDCoV HKU15), White-eye CoV HKU16 (WECoV HKU16), Sparrow CoV HKU17 (SpCoV HKU17), Magpie Robin CoV HKU18 (MRCoV HKU18), Night heron CoV HKU19 (NHCoV HKU19), wigeon CoV HKU20 (WiCoV HKU20), Common moorhen CoV HKU21 (CMCoV HKU21), falcon CoV HKU27 (FalCoV UAE-HKU27), houbara bustard CoV HKU28 (HouCoV UAE-HKU28), pigeon CoV HKU29 (PiCoV UAE-HKU29), and quail CoV HKU30 (QuaCoV UAE-HKU30), which are the best characterized DCoV species. Delta coronaviruses are described in further detail in Vlasova et al. (2021) Frontiers in Veterinary Science, Vol. 10, doi: 10.3389/fvets.2020.626785. Non-human coronaviruses are described in further detail in Kenney et al. 2020 Veterinary Pathology, Vol. 58, Issue 3, pages 438-452, doi: 10.1177/0300985820980842; Alluwaimi et al. (2020) Frontiers in Veterinary Science, Vol. 7, Article number 582287, doi: 10.3389/fvets.2020.582287).

C. Methods of Administration

The methods for reducing the replication of a coronavirus infection, or methods for achieving a desired alleviation of coronavirus-associated disease symptoms, include administering to an animal, such as a mammal, especially a human being, an effective amount of a combination of a pharmaceutical composition comprising one or more caspase-6 inhibitor (s) and optionally one or more therapeutic, prophylactic or diagnostic agents, such as part of the same composition, or administered separately and independently at the same time or at different times (i.e., administration of the one or more therapeutic, prophylactic or diagnostic agents, and the one or more caspase-6 inhibitor (s) is separated by a finite period of time from each other). Therefore, the term “combination” or “combined” is used to refer to either concomitant, simultaneous, or sequential administration of the one or more caspase-6 inhibitor (s) and one or more optional therapeutic, prophylactic or diagnostic agents. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

1. Effective Amounts

Formulations of including caspase-6 inhibitor (s) and one or more therapeutic, prophylactic, and/or diagnostic agents typically include an effective amount of an admixture of the one or more caspase-6 inhibitor (s) and one or more therapeutic, prophylactic, and/or diagnostic agents. Effective amounts of the combined caspase-6 inhibitor (s) are provided herein. It will be appreciated that in some forms the effective amount of the one or more caspase-6 inhibitor (s) and one or more therapeutic, prophylactic, and/or diagnostic agents is different from the amount that would be effective for the one or more therapeutic, prophylactic, and/or diagnostic agents to achieve the same result when administered in the absence of the caspase-6 inhibitor (s).

When used for treating a coronavirus infection in a subject, the amount of caspase-6 inhibitor (s) present in a pharmaceutical dosage unit, or otherwise administered to a subject, can be the amount effective to reduce the production, inhibit the activation, or inhibit a signaling pathway of caspase-6. In some forms, the amount of caspase-6 inhibitor (s) present in a pharmaceutical dosage unit, or otherwise administered to a subject, can be the amount effective to reduce coronavirus replication in small airways and alveoli of the lungs. Preferably, the amount of caspase-6 inhibitor (s) present in the pharmaceutical dosage unit, is administered to the subject in an amount effective to reduce coronavirus replication by 50% or more 24 hours following administration.

In some forms, the amount of caspase-6 inhibitor (s) present in a pharmaceutical dosage unit, or otherwise administered to a subject, can be the amount effective to reduce bronchiolitis, alveolitis, and vasculitis. In some forms, the amount of caspase-6 inhibitor (s) present in a pharmaceutical dosage unit, or otherwise administered to a subject, can be the amount effective to reduce to reduce bronchiolar epithelial cell death and desquamation, alveolar space mononuclear cell infiltration, protein rich fluid exudation, alveolar hemorrhage, damage to alveolar structure, pulmonary blood vessel wall inflammation and endothelium infiltration, focal alveolar septal congestion and perivascular infiltration, and lower alveolar space immune cells. In some forms, the amount of caspase-6 inhibitor (s) present in a pharmaceutical dosage unit, or otherwise administered to a subject, can be the amount effective to reduce body weight loss and improve survival.

In some forms, the amount of caspase-6 inhibitor (s) present in a pharmaceutical dosage unit, or otherwise administered to a subject, can be the amount effective to reduce the expression of one or more pro-inflammatory cytokines and/or chemokines selected from the group comprising IL6, IL1β, TNFα, IFNγ, IP10, IP6, MCP1, CXCL3, CXCL5, and/or CXCL9. In some forms, the amount of caspase-6 inhibitor (s) present in a pharmaceutical dosage unit, or otherwise administered to a subject, can be the amount effective to upregulates the expression of one or more IFN-signaling genes selected from the group comprising IFIT1, IFIT2, IFIT3, IFITM3, TRIM22, and/or OAS1 following treatment.

In some forms, the pharmaceutical composition containing the caspase-6 inhibitor (s) is administered to a subject in need thereof, to deliver the caspase-6 inhibitor in an amount between about 0.1 mg and about 1,000 mg, inclusive, preferably between about 0.5 mg and about 100 mg, inclusive, more preferably between about 1 mg and about 15 mg, inclusive, for example, 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, and/or 15 mg.

2. Dosage Regimens

A dosage regimen of the pharmaceutical composition containing the one or more caspase-6 inhibitor (s) and optionally one or more therapeutic, prophylactic, and/or diagnostic agents, can include one or multiple administrations of the pharmaceutical composition.

In some forms, the pharmaceutical compositions are administered at least 1, 2, 3, 4, 5, 6, 7, 8, 9, 10, 11, 12, 13, 14, 15, 16, 17, 18, 19, 20, 21, 22, 23, 24 or 30 hours, or more than 30 hours, up to 36 or 48 hours prior to or after the detection of the coronavirus in the patient. In other forms, the pharmaceutical compositions are administered at least 1, 2, 3, 4, 5, 6, 7, 8, 9, 10, 11, 12, 13, 14, 15, 16, 17, 18, 19, 20, 21, 22, 23, 24 or 30 hours, or more than 30 hours, up to 36 or 48 hours prior to or after administering a separate therapeutic, prophylactic, or diagnostic agent. In certain forms, additive or more than additive effects of the administration of the pharmaceutical composition containing the one or more caspase-6 inhibitor (s) in combination with one or more therapeutic and/or prophylactic agent (s) is evident after one day, two days, three days, four days, five days, six days, one week, two weeks, three weeks, or more than three weeks following administration.

An effective amount of the pharmaceutical compositions and optionally one or more therapeutic and/or prophylactic agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

D. Subjects to Be Treated

A subject in need of treatment is a subject having coronavirus disease or a subject having or at risk of having a coronavirus-associated disease. Exemplary diseases associated with coronavirus infections include respiratory diseases, such as coronavirus-induced pneumonia, coronavirus-induced bronchitis, acute respiratory distress syndrome (ARDS), acute lung injury (ALI), multisystem inflammatory syndrome in children (MIS-C), and/or multisystem inflammatory syndrome in adults (MIS-A). In some forms, the subject is a mammal, including, but not limited to, murines, simians, humans, mammalian farm animals and livestock, mammalian sport animals, and mammalian pets. Preferably, the subject is a human.

A subject having a coronavirus infection is a subject that has been exposed to a coronavirus and has acute or chronic detectable levels of the coronavirus in his/her body or has signs and symptoms associated with infection of the coronavirus. Methods of assessing and detecting coronavirus infections in a subject are known by those of ordinary skill in the art. A subject at risk of having a coronavirus infection is a subject that may be expected to come in contact with a coronavirus as described above. Examples of such subjects are medical workers or those traveling to parts of the world where the incidence of infection is high. In some forms, the subject is at an elevated risk of an infection because the subject has one or more risk factors to have an infection. Examples of risk factors to be infected and/or develop mild, moderate, and/or severe symptoms include immunosuppression, immunocompromised, age (advanced or very young), and surgery. The degree of risk of infection depends on the multitude and the severity or the magnitude of the risk factors that the subject has. Risk charts and prediction algorithms are available for assessing the risk of an infection in a subject based on the presence and severity of risk factors. Other methods of assessing the risk of infection in a subject are known by those of ordinary skill in the art. In some forms, the subject who is at an elevated risk of an infection may be an apparently healthy subject. An apparently healthy subject is a subject who has no signs or symptoms of disease.

The effect of the pharmaceutical compositions including one or more caspase-6 inhibitor (s) can be compared to a control. Suitable controls are known in the art and include, for example, an untreated subject, or a placebo-treated subject. A typical control is a comparison of a condition or symptom of a subject prior to and after administration of the pharmaceutical compositions including one or more caspase-6 inhibitor (s). The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some forms, the symptom, pharmacologic, or physiologic indicator is measured in a subject prior to treatment, and again one or more times after treatment is initiated. In some forms, the control is a reference level, or average determined based on measuring the symptom, pharmacologic, or physiologic indicator in one or more subjects that do not have the disease or condition to be treated (e.g., healthy subjects). In some forms, the effect of the treatment is compared to a conventional treatment that is known the art. Suitable control subjects are unvaccinated subjects, or subjects receiving the same amount of a therapeutic, prophylactic and/or diagnostic agent in the absence of pharmaceutical compositions including one or more caspase-6 inhibitor(s).

E. Routes of Administration

The disclosed pharmaceutical compositions containing one or more caspase-6 inhibitors in an amount sufficient to reduce the replication of a coronavirus in a subject and ameliorate symptoms associated with a coronavirus infection are typically administered according to methods known for administering vaccines to subjects.

In some forms, the disclosed pharmaceutical composition containing one or more caspase-6 inhibitors are administered parenterally. The phrases “parenteral administration” and “administered parenterally” are art-recognized terms, and include modes of administration other than enteral and topical administration, such as injections, and include without limitation intravenous, intramuscular, intrapleural, intravascular, intrapericardial, intraarterial, intrathecal, intratracheal, intranasal intracapsular, intraorbital, intracardiac, intradennal, intraperitoneal, transtracheal, subcutaneous, subcuticular, intraarticular, subcapsular, subarachnoid, intraspinal and intrastemal injection and infusion. The disclosed pharmaceutical composition containing one or more caspase-6 inhibitors can be administered parenterally, for example, by subdural, intravenous, intrathecal, intraventricular, intraarterial, intra-amniotic, intraperitoneal, or subcutaneous routes. In preferred forms, the disclosed pharmaceutical composition containing one or more caspase-6 inhibitors are administered via oral, intranasal, intraperitoneal, intratracheal, or intrathecal administration.

### IV. Kits

Medical kits are also disclosed. The medical kits can include, for example, a dosage supply of pharmaceutical composition containing one or more caspase-6 inhibitors, and optionally one or more therapeutic, prophylactic or diagnostic agents, either separately or together in the same admixture. The active agents can be supplied alone (e.g., lyophilized), or in a pharmaceutical composition. The active agents can be in a unit dosage, or in a stock that should be diluted prior to administration. In some forms, the kit includes a supply of pharmaceutically acceptable carrier. The kit can also include devices for administration of the active agents or compositions, for example, syringes. The kits can include printed instructions for administering the compound in a use as described above.

The disclosed compositions and methods can be further understood through the following numbered paragraphs.

1. A method of treating a coronavirus infection in a subject comprising administering a pharmaceutical composition comprising an effective amount of a caspase-6 inhibitor to the subject in need thereof, wherein the amount of the caspase-6 inhibitor in the pharmaceutical composition when administered to the subject in need thereof, is effective to reduce replication of the coronavirus in the subject.

2. A method of preventing or treating a coronavirus-associated disease in a subject comprising administering to the subject, an effective amount of the pharmaceutical composition of paragraph 1.

3. The method of paragraphs 1 or 2, wherein the amount of the caspase-6 inhibitor in the pharmaceutical composition is effective, when administered to the subject, to reduce the replication of the coronavirus in the subject compared to an untreated subject with a coronavirus infection.

4. The method of any of paragraphs 1-3, wherein the caspase-6 inhibitor comprised in the pharmaceutical composition does not inhibit one or more other caspases.

5. The method of any of paragraphs 1-4, wherein the caspase-6 inhibitor comprised in the pharmaceutical composition is not a pan caspase inhibitor.

6. The method of any of paragraphs 1-5, wherein the caspase-6 inhibitor comprised in the pharmaceutical composition is Z-VEID-FMK or Ac-VEID-CHO.

7. The method of any of paragraphs 1-6, wherein the caspase-6 inhibitor comprised in the pharmaceutical composition is Z-VEID-FMK.

8. The method of any of paragraphs 1-5, wherein the caspase-6 inhibitor comprised in the pharmaceutical composition is a nucleic acid molecule selected from the group comprising a single stranded antisense nucleic acid (ssRNA), a small interfering NA (siRNA), a short hairpin RNA (shRNA), and a microRNA (miRNA).

9. The method of any of paragraphs 1-7, wherein the amount of the caspase-6 inhibitor in the pharmaceutical composition is effective, when administered to the subject, to reduce coronavirus replication by 50% or more 24 hours following administration.

10. The method of any of paragraphs 1-9, wherein the pharmaceutical composition is effective, when administered to the subject, to deliver the caspase-6 inhibitor at a dose from about 7.5 mg/kg/day or more.

11. The method of any of paragraphs 1-10, wherein the pharmaceutical composition is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof following the detection of the coronavirus in the subject.

12. The method of any of paragraphs 1-11, wherein the pharmaceutical composition is administered via oral, intranasal, intraperitoneal, intratracheal, or intrathecal administration.

13. The method of any of paragraphs 1-12, wherein the coronavirus is an alpha coronavirus, a beta coronavirus, a gamma coronavirus, or a delta coronavirus.

14. The method of any of paragraphs 1-13, wherein the coronavirus is selected from the group comprising Human Coronavirus 229E (HCoV-229E), Human Coronavirus OC43 (HCoV-OC43), Human Coronavirus NL63 (HCoV-NL63), Human Coronavirus HKU1 (HCoV-HKU1), Middle East Respiratory Syndrome Coronavirus (MERS-CoV), Severe Acute Respiratory Syndrome Coronavirus 1 (SARS-CoV-1), and SARS-CoV-2.

15. The method of any of paragraphs 1-12, wherein the coronavirus is a non-human coronavirus.

16. The method of any of paragraphs 1-15, wherein the coronavirus is a pathogenic coronavirus.

17. The method of paragraph 16, wherein the pathogenic coronavirus is selected from the group comprising Middle East Respiratory Syndrome Coronavirus (MERS-CoV), Severe Acute Respiratory Syndrome Coronavirus 1 (SARS-CoV-1), and SARS-CoV-2.

18. The method of any of paragraphs 14-17, wherein the SARS-CoV-2 variant is the alpha variant, beta variant, gamma variant, delta variant, epsilon variant, eta variant, iota variant, kappa variant, mu variant, omicron variant, zeta variant, 1.617.3 variant and/or lambda variant.

19. The method of any of paragraphs 14 or 16-18, wherein the SARS-CoV-2 variant is a sub-variant of the alpha variant, beta variant, gamma variant, delta variant, epsilon variant, eta variant, iota variant, kappa variant, mu variant, omicron variant, zeta variant, 1.617.3 variant and/or lambda variant.

20. The method of any of paragraphs 1-19, wherein the subject is a human.

21. The method of any of paragraphs 1-20, wherein the subject has a coronavirus-induced pneumonia, coronavirus-induced bronchitis, acute respiratory distress syndrome (ARDS), acute lung injury (ALI), multisystem inflammatory syndrome in children (MIS-C), and/or multisystem inflammatory syndrome in adults (MIS-A).

22. The method of any of paragraphs 1-21, wherein the subject is immunocompromised.

23. A pharmaceutical composition for the treatment of a coronavirus infection in a subject comprising an effective amount of a caspase-6 inhibitor, wherein the amount of caspase-6 inhibitor in the composition is effective, when administered to the subject, to reduce replication of coronavirus in the subject.

24. The pharmaceutical composition of paragraph 23, wherein the amount of caspase-6 inhibitor in the composition is effective, when administered to the subject, to reduce replication of coronavirus in the subject compared to an untreated subject with a coronavirus infection.

25. The pharmaceutical composition of paragraphs 23 or 24, wherein the caspase-6 inhibitor does not inhibit one or more other caspases such as caspase-8.

26. The pharmaceutical composition of any of paragraphs 23-25, wherein the caspase-6 inhibitor is not a pan caspase inhibitor.

27. The pharmaceutical composition of any of paragraphs 23-26, wherein the caspase inhibitor is Z-VIED-FMK or Ac-VEID CHO.

28. The pharmaceutical composition of any of paragraphs 23-27, wherein the caspase inhibitor is Z-VIED-FMK.

29. The pharmaceutical composition of any of paragraphs 23-28, wherein the amount of the caspase-6 inhibitor in the composition is effective, when administered to the subject, to reduce coronavirus replication by 50% or more 24 hours following administration.

### EXAMPLES

### Example 1: Inhibition of Caspase-6 Limits Coronavirus Replication

Materials and Methods

Cell Lines

A549, BSC-1, Caco2, Huh7, VeroE6, and 293T cells were maintained in Dulbecco's Modified Eagle medium (DMEM) supplemented with 10% heat-inactivated fetal bovine serum (FBS), 100 unit/ml penicillin, and 100 g/ml streptomycin (Chu, H. et al. (2020) Lancet Microbe vol. 1(1), e14-e23, doi:10.1016/S2666-5247(20)30004-5; Chan, J. F. et al. (2013) Journal of Infectious Diseases vol. 207(11), pages 1743-1752 doi:10.1093/infdis/jitl23). BEAS2B and Calu3 cells were maintained in DMEM/F12 supplemented with 10% heat inactivated FBS, 100 unit/ml penicillin, and 100 μg/ml streptomycin. HFL (primary human embryonic lung fibroblast) cells were maintained in Minimum Essential Medium (MEM) supplemented with 10% heat inactivated FBS, 100 unit/ml penicillin and 100 μg/ml streptomycin. Human primary monocytes were obtained from human peripheral blood mononuclear cells (PBMCs) taken from healthy donors, collected from the Hong Kong Red Cross Blood Transfusion Service according to a protocol approved by the Institutional Review Board of the University of Hong Kong (Chu, H. et al. (2014) Virology Vol. 454-455, pages 197-205, doi: 10.1016/j.virol.2014.02.018). Primary human monocyte-derived macrophages (MDMs) were differentiated from monocytes in Roswell Park Memorial Institute (RPMI)—1640 media supplemented with 10% heat-inactivated FBS, 100 unit/ml penicillin, 100 μg/ml streptomycin, 2 mM glutamine, 1% sodium pyruvate, 1% non-essential amino acids, and 10 ng/ml recombinant human granulocyte macrophage colony-stimulating factor (GM-CSF) (R&D Systems) as described in previous studies (Chu, H. et al. (2012) Cell Host Microbe vol. 12(3), pages 360-372, doi: 10.1016/j.chom.2012.07.011).

Viruses

The MERS-CoV (EMC/2012) strain of MERS-CoV was obtained from Erasmus Medical Center. The mouse-adapted MERS-CoV (MERS-CoV MA) was obtained from University of Iowa. SARS-CoV-2 wildtype HKU-001a50, Omicron BA.1 (GenBank: OM212472), and Omicron BA.2 (GISAID: EPI_ISL_9845731) were isolated from a laboratory-confirmed COVID-19 patient in Hong Kong. SARS-CoV-1 GZ50, HCoV-229E, HCoV-OC43, enterovirus A71, and influenza A virus strain A/Hong Kong/415742/2009(H1N1)pdm09 were archived clinical isolates at Department of Microbiology, University of Hong Kong (HKU, Yuan, S. et al. (2019) Nature Communications, Vol. 10(1), Article 120, doi: 10.1038/s41467-018-08015-x; Yuan, S. et al. (2020) Science Advances 6, Article 35, eaba7910, doi: 10.1126/sciadv.aba7910). All infectious experiments involving MERS-CoV, SARS-CoV-2, and SARS-CoV-1 followed the approved standard operating procedures of the Biosafety Level 3 facility at the Department of Microbiology, HKU.

Chemical Modulators

The pan-caspase inhibitor, z-VAD-fmk, was obtained from InvivoGen (Catalog #tlrl-vad). The caspase-1-to-caspase-10 inhibitor sampler kit was purchased from R&D Systems. The caspase-6 inhibitors, z-VEID-fmk, used for in vitro and in vivo experiments, were obtained from R&D Systems and APExBIO, respectively. The apoptosis enhancer, Staurosporine, was obtained from Sigma-Aldrich. Filgotinib (JAK1 inhibitor), Ruxolitinib (JAK1/2 inhibitor), and IFN alpha-IFNAR-IN-1 hydrochloride were obtained from MedChemExpress.

Antibodies

MERS-CoV N, MERS-CoV Spike, SARS-CoV-1 N and SARS-CoV-2 N were detected with specific in-house immune serum. Primary antibodies including rabbit anti-caspase-3, rabbit anti-caspase-6, rabbit anti-HA, mouse anti-Flag, mouse anti-His, and mouse anti-3-actin were from Abcam. Secondary antibodies including goat anti-mouse horseradish peroxidase (HRP), goat anti-rabbit HRP and goat anti-guinea pig HRP from ThermoFisher Scientific were used for Western blots. Alexa Fluor 488 goat anti-guinea pig, Alexa Fluor 488 goat anti-rabbit and Alexa Fluor 568 goat anti-rabbit from ThermoFisher Scientific were used for immunohistochemistry staining.

Ex Vivo Human Lung Tissues

Ex vivo human lung tissues were processed and infected with MERS-CoV as described in previous studies (Chu, H. et al. (2020) Clinical Infectious Diseases Vol. 71(6), pages 1400-1409, doi: 10.1093/cid/ciaa410; Zhou, J. et al. (2014) Journal of Infectious Diseases Vol. 209, pages 1331-1342, doi: 10.1093/infdis/jit504). Human lung tissues for ex vivo studies were retrieved from patients who underwent surgical operations. All donors gave written consent as approved by the Institutional Review Board. Normal non-malignant lung tissue fragments in excess for clinical diagnosis were used. The freshly obtained lung tissues were processed into small rectangular pieces and were rinsed with the primary tissue culture medium, which contained the advanced DMEM/F12 medium supplemented with 2 mM HEPES (Gibco™), 1× GlutaMAX (Gibco™), 100 unit/ml penicillin, 100 μg/ml streptomycin, 20 μg/ml vancomysin, 20 g/ml ciprofloxacin, 50 μg/ml amikacin, and 50 μg/ml nystatin. The specimens were infected with MERS-CoV at a titer of 1×10⁸ PFU/ml. After 2 hours, the inoculum was removed, and the specimens were washed thoroughly with the primary tissue culture medium. The infected tissues were then incubated with primary tissue culture medium supplemented with 100 μM caspase-6 inhibitor z-VEID-fmk dissolved in DMSO or DMSO only. Tissues were harvested at 24 hpi (human protein index) with either 10% neutral-buffered formalin for immunofluorescence staining or with RL buffer for RT-qPCR analysis.

Human Intestinal Organoids

Human Intestinal organoids were established using biopsied human intestinal tissues from patients who underwent surgical operations at the Queen Mary Hospital, Hong Kong (Zhou, J. et al. (2020) Nature Medicine Vol. 26(7), pages 1077-1083, doi:10.1038/s41591-020-0912-6). All donors had written consent as approved by the Institutional Review Board. Human intestinal organoids were maintained in expansion medium and induced differentiation by incubating with differentiation media for 5 days described in previous studies (Zhou, J. et al. (2020) Nature Medicine Vol. 26(7), pages 1077-1083, doi:10.1038/s41591-020-0912-6). Differentiated intestinal organoids were sheared mechanically and inoculated with MERS-CoV at 1 MOI (multiplicity of infection) for 2 hours. After the inoculum was removed, the intestinal organoids were rinsed with PBS, embedded in Matrigel, and maintained in differentiation medium containing 100 μM z-VEID-fmk. At the indicated time points following inoculation, intestinal organoids were harvested for the quantification of intracellular viral load and immunofluorescence staining, whereas the cell-free Matrigel and culture medium were combined for viral titration of extracellular virions using standard plaque assays.

Human DPP4 Mouse Model

The human dipeptidyl peptidase 4 Knock-In (hDPP4 KI) mice were obtained from the University of Iowa (Li, K. et al. (2017) Proceedings of the National Academy of Sciences of the United States of America Vol. 114(15), E3119-E3128, doi:10.1073/pnas.1619109114). The use of animals complied with all relevant ethical regulations and was approved by the Committee on the Use of Live Animals in Teaching and Research of The University of Hong Kong. On the day of infection, hDPP4 KI mice were intranasally (i.n.) inoculated with 2.5×10³ PFU of mouse-adapted MERS-CoV (MERS-CoV MA) pre-diluted in 20 μl DMEM, followed by intraperitoneal (i.p.) injection with 12.5 mg/kg/day z-VEID-fmk or DMSO diluted in 200 μl 0.3% methylcellulose/0.1% tween-80/PBS once per day for 6 days or until sample harvest. The health status and body weight of the mice were monitored daily for 14 days or until the animal is sacrificed or euthanized to facilitate a humane endpoint in the experiment. Mice were sacrificed at the designated time points and lung tissue from mice of both treatment and control groups were harvested for immunofluorescence staining, RT-qPCR, and plaque assay analysis.

Golden Syrian Hamster Model

Golden Syrian hamsters were infected using protocols described in previous studies (Chan, J. F. et al. (2020) Clinical Infectious Diseases Vol. 71(9), pages 2428-2446, doi:10.1093/cid/ciaa325). Golden Syrian hamsters aged 6-8 weeks old were obtained from the Chinese University of Hong Kong Laboratory Animal Service Centre through the HKU Centre for Comparative Medicine Research (CCMR). The use of animals complied with all relevant ethical regulations and was approved by the Committee on the Use of Live Animals in Teaching and Research of The University of Hong Kong. On the day of infection, each hamster was intranasally inoculated with 3×10³ PFU SARS-CoV-2 pre-diluted in 50 μl DMEM under intraperitoneal ketamine (100 mg/kg) and xylazine (10 mg/kg) induced-anesthesia. Infected hamsters were treated with 12.5 mg/kg/day z-VEID-fmk or DMSO diluted in 600 μl 0.3% methylcellulose/0.1% tween-80/PBS once per day for 4 days. The health status and body weight of the hamsters were monitored daily or until the animal is sacrificed or euthanized. Hamsters were sacrificed at day 4 post infection, and lung tissues were harvested for immunofluorescence staining, histopathology examination, RT-qPCR, and Tissue Culture Infectious Dose 50 (TCID50) assay analysis (Chan, J. F. et al. (2020) Clinical Infectious Diseases Vol. 71(16), pages 2139-2149, doi:10.1093/cid/ciaa644 (2020); Yuan, S. et al. (2020) Nature Microbiology Vol. 5(11), pages 1439-1448, doi:10.1038/s41564-020-00802-x).

Generation and Infection of Caspase-6 Knockout Mice

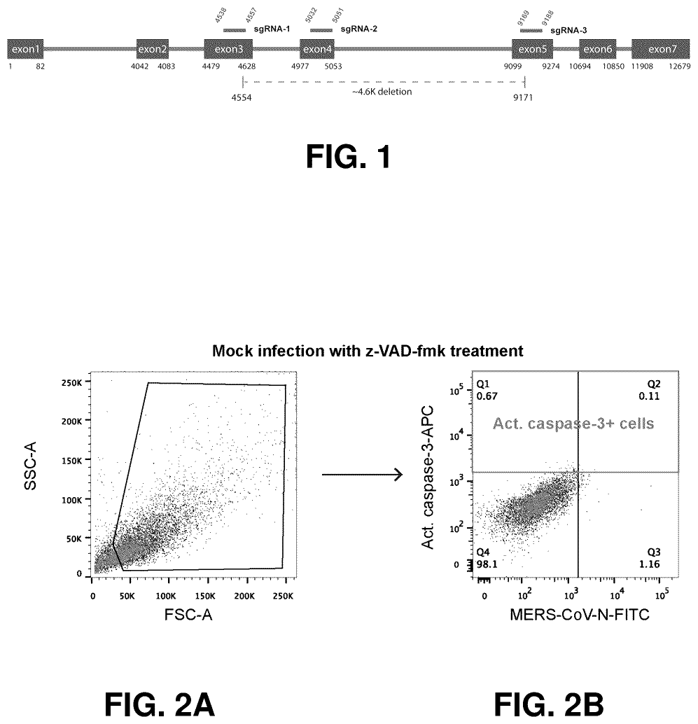

Generation of caspase-6 KO mice in the background of hDPP4 KI mice were bred. Briefly, the following three sgRNA were designed to target caspase-6 exon 3, exon 4, and exon 5 respectively, and were purchased from Synthego (FIG. 1):

(SEQ ID NO: 1)
1) 5′-CCCTCATCTTCAATCACGAG AGG-3′,
(SEQ ID NO: 2)
2) 5′-CTCCTGCTCAAAATTCACGA GGG-3′,
and
(SEQ ID NO: 3)
3) 5′-TGGCGTCGTATGCGTAAACG TGG-3′

The Cas9 Protein was purchased from Invitrogen (TrueCut Cas9 Protein version 2). First, the sgRNAs (0.5 μg/μl) and Cas9 protein (0.4 μg/μl) were diluted in Opti-MEM (Gibco™). Formation of the ribonucleoprotein (RNP) was facilitated via vortexing the reagent mixture, followed by 20 minutes of incubation at room temperature. Second, in vitro fertilization (IVF) was performed to generate homozygous hDPP4 embryos required for generating double mutant by electroporation. Five-week-old homozygous hDPP4 females were super-ovulated using an i.p. injection of 7.5 international units (IU) pregnant mare serum gonadotropin (PMSG), followed by an i.p injection of 7.5 IU human chorionic gonadotropin (hCG) 48 hours later. 12 hours following the hCG injection, 3-month-old homozygous hDPP4 stud males were sacrificed. Sperm was released from the cauda epididymis into one well of a 4-well plate. The sperm was allowed to capacitate in modified human tubal fluid medium (mHTF) for 45 minutes in an incubator (5% CO2, 37° C.). Oocytes cumuli were collected from the oviducts of the super-ovulated females and were added into the sperm well. Four hours following the IVF, embryos were washed twice with mHTF medium and twice with potassium(K)-supplemented simplex optimized medium (KSOM). Embryos were cultured in KSOM in an incubator until the electroporation. Third, the RNP was delivered into pronuclear stage embryos using a square wave electroporator (CUY21 EDIT II, Bex Co. Ltd). 5 μl of the RNP was pipetted into the chamber of the platinum electrode with a 1 mm gap (LF501PT1-5, Bex Co. Ltd.). The parameters were set as followed, (i) five poring Pulses: 200 V, 2 milliseconds length, 50 milliseconds interval, ×4 times, 10% voltage decay, +polarity, followed by (ii) five transfer Pulses of 20 V, 50 milliseconds length, 50 milliseconds interval, ×40% voltage decay, alternating +/−polarity. The impedance was adjusted below 0.17Ω by adding a small volume of Opti-mem into the gap. The electroporated embryos were immediately transferred back to KSOM and were incubated for 30 minutes. The embryos were washed twice with fresh KSOM and were incubated overnight. The following morning, two-cell embryos were transferred into the oviduct of 0.5-day post coitum (dpc) pseudo-pregnant ICR (CD-1) females. 20 days after the embryo transfer, caspase-6 KO pups were born. The pups were weaned at 3 weeks of age. Ear punch samples were collected for caspase 6 genotyping. On the day of infection, hDPP4 KI caspase-6 KO mice and hDPP4 KI caspase-6 WT mice were intranasally (i.n.) inoculated with 2.5×10³ PFU MERS-CoV MA pre-diluted in 20 l DMEM. Mice were sacrificed at 2 dpi (days post inoculation) and 4 dpi, and lung tissues from mice of both groups were harvested for RT-qPCR, plaque assay analysis, H&E (hematoxylin and eosin) staining, and immunofluorescence staining.

Generation and Infection of MERS-CoV Recombinant Virus

The cDNA from the MERS-CoV strain EMC/2012 (GenBank accession number JX869059), assembled into the pBAC (pBAC-SA-FL), was used as the background to generate the transketoase A (TKKA) mutant (rMERS-CoV/TKKA) with a D to A change at the TKKD motif of the MERS-CoV N gene (Almazan, F. et al. (2013) American Society for Microbiology Vol. 4(5), e00650-00613, doi:10.1128/mBio.00650-13). The mutation was introduced into the pBAC by red recombineering (Almazan, F., et al. (2015) Methods in Molecular Biology Vol. 1282, pages 135-152, doi:10.1007/978-1-4939-2438-7-13). The mutation was confirmed by Sanger sequencing. The recombinant clones with the mutant site were transformed into DH10B electrocompetent cells, and the plasmids were extracted to acquire ultrapure and high-quality full-length cDNA clones. The infectious virus was recovered via transfection of BHK21 cells with 5 μg of the full-length cDNA clone using Lipofectamine 3000 as a transfection reagent. Six hours post-transfection, the transfected BHK21 cells were re-seeded and co-cultured with Huh7 cells. After 72 hours, the supernatant was used to inoculate Huh7 cells for viral passage. The recombinant virus sequenced using next generation sequencing (NGS) to confirm the desired mutation and the absence of undesired mutations in the viral genome. For in vitro infection, Huh7, A549, and VeroE6 cells were infected with rMERS-CoV/TKKA and rMERS-CoV/TKKD. Cell lysate and supernatant samples were harvested from the infected cells at 2 hours post infection (hpi), 24 hpi, and 48 hpi for RT-qPCR and TCID50 assays, respectively. Additionally, rMERS-CoV/TKKA- and rMERS-CoV/TKKD-infected Huh7 cells were harvested at 1 hpi, 24 hpi, and 48 hpi for Western blot analysis of N protein expression. For in vivo infection, hDPP4 KI mice were intranasally inoculated with 3.5×10⁴ PFU rMERS-CoV/TKKA or rMERS-CoV/TKKD pre-diluted in 20 l DMEM. Mice were sacrificed at 2 dpi and 4 dpi and lung tissues from mice of both groups were harvested for RT-qPCR, plaque assay analysis, H&E staining, and immunofluorescence staining.

Caspase-6 CRISPR/Cas9 Knockout In Vitro

Caspase-6 CRISPR/Cas9 KO plasmids were purchased from Santa Cruz Biotechnology. Knockout of caspase-6 in Huh7 cells was achieved by co-transfection with HDR plasmid according to company's protocol (datasheets.scbt.com/protocols/CRISPR_protocol). In brief, Huh7 cells were seeded in 6-well plate, followed by transfected with 3 μg caspase-6 CRISPR/Cas9 KO plasmids and 3 μg HIDR plasmid, or non-targeting control plasmids using Lipofectamine 3000. At 72 hours post-transfection, caspase-6 knockout targeting cells were selected by medium containing 1.2 μg/mL puromycin. Caspase-6 KO was verified with Western blots. On the day of infection, Huh7 cells were infected by MERS-CoV at 1 MO. At 24 hpi and 48 hpi, the cell lysates and supernatants were harvested for RT-qPCR quantification and TCID50 assays.

RNA Extraction and Quantitative RT-PCR

Cells were lysed in RL buffer and extracted with the MiniBEST Universal RNA Extraction Kit (TaKaRa). Viral RNA in the supernatant was extracted with the MiniBEST Viral RNA/DNA Extraction Kit (TaKaRa). Reverse transcription (RT) and quantitative polymerase chain reaction (qPCR) were performed with Transcriptor First Strand cDNA Synthesis Kit and LightCycler 480 master mix from Roche. All primer and probe sequences are provided in Table 2.

TABLE 2
List of RT-qPCR Primers

| | | Sequence | |
|---|---|---|---|
| Species | Gene | (5′-3′) | |
| EV-A71 | VP1 | F | GCCCCTGAATGCGGCTAAT |
| | | | (SEQ ID NO: 9) |
| | | R | ATTGTCACCATAAGCAGYCA |
| | | | (SEQ ID NO: 10) |
| | | Probe 5′-FAM-CGGACACCCAAAGTAGTCG | |
| | | GTTCCG- 1ABKFQ-3′ | |
| | | (SEQ ID NO: 11) | |
| H1N1 | M | F | CTTCTAACCGAGGTCGAAACG |
| | | | (SEQ ID NO: 13) |
| | | R | GGCATTTTGGACAAAKCGTCTA |
| | | | (SEQ ID NO: 14) |
| HCoV- | N | F | CGTACTCCTAAGCCTTCTCG |
| 229E | | | (SEQ ID NO: 15) |
| | | R | TCGACTAGGGTTAAGAAGAGG |
| | | | (SEQ ID NO: 16) |
| HCoV- | N | F | CGATGAGGCTATTCCGACTAGGT |
| OC43 | | | (SEQ ID NO: 17) |
| | | R | CCTTCCTGAGCCTTCAATATAGTAA |
| | | | CC |
| | | | (SEQ ID NO: 18) |
| MERS- | N | F | CAAAACCTTCCCTAAGAAGGAAAAG |
| CoV | | | (SEQ ID NO: 19) |
| | | R | GCTCCTTTGGAGGTTCAGACAT |
| | | | (SEQ ID NO: 20) |
| SARS- | N | F | ACCAGAATGGAGGACGCAATG |
| CoV-1 | | | (SEQ ID NO: 21) |
| | | R | GCTGTGAACCAAGACGCAGTATTAT |
| | | | (SEQ ID NO: 22) |
| SARS- | RdRp | F | CGCATACAGTCTTRCAGGCT |
| CoV-2 | | | (SEQ ID NO: 23) |
| | | R | GTGTGATGTTGAWATGACATGGTC |
| | | | (SEQ ID NO: 24) |
| | | Probe 5′- FAM-TTAAGATGTGGTGCTTGCA | |
| | | TACGTAGAC- 1ABKFQ-3′ | |
| | | (SEQ ID NO: 25) | |
| Human | 2′5′- | F | AGGAAAGGTGCTTCCGAGGTAG |
| | OAS1 | | (SEQ ID NO: 12) |
| | | R | GGACTGAGGAAGACAACCAGGT |
| | | | (SEQ ID NO: 26) |
| | GAPDH | F | ATTCCACCCATGGCAAATTC |
| | | | (SEQ ID NO: 27) |
| | | R | CGCTCCTGGAAGATGGTGAT |
| | | | (SEQ ID NO: 28) |
| | IFIT1 | F | AGAAGCAGGCAATCACAGAAAA |
| | | | (SEQ ID NO: 29) |
| | | R | CTGAAACCGACCATAGTGGAAAT |
| | | | (SEQ ID NO: 30) |
| | IFIT2 | F | CACATGGGCCGACTCTCAG |
| | | | (SEQ ID NO: 31) |
| | | R | CCACACTTTAACCGTGTCCAC |
| | | | (SEQ ID NO: 32) |
| | IFIT3 | F | GAACATGCTGACCAAGCAGA |
| | | | (SEQ ID NO: 33) |
| | | R | CAGTTGTGTCCACCCTTCCT |
| | | | (SEQ ID NO: 34) |
| | IFITM3 | F | ATGTCGTCTGGTCCCTGTTC |
| | | | (SEQ ID NO: 35) |
| | | R | GTCATGAGGATGCCCAGAAT |
| | | | (SEQ ID NO: 36) |
| | IFNß | F | CGCCGCATTGACCATCTA |
| | | | (SEQ ID NO: 37) |
| | | R | GACATTAGCCAGGAGGTTCT |
| | | | (SEQ ID NO: 38) |
| | TRIM22 | F | ACTGTCTCAGGAACACCAAGGTCA |
| | | | (SEQ ID NO: 39) |
| | | R | CCAGGTTATCCAGCACATTCACCTCA |
| | | | (SEQ ID NO: 40) |
| hDPP4 | CXCL5 | F | CGGTTCCATCTCGCCATTCA |
| KI | | | (SEQ ID NO: 41) |
| mice | | R | GCGGCTATGACTGAGGAAGG |
| | | | (SEQ ID NO: 42) |
| | CXCL9 | F | GAGTGGAAGTGTGGACAGGG |
| | | | (SEQ ID NO: 43) |
| | | R | TCAAGACGGAACTTCTGCCC |
| | | | (SEQ ID NO: 44) |
| | GAPDH | F | CGACTTCAACAGCAACTCCCACTCT |
| | | | TCC |
| | | | (SEQ ID NO: 45) |
| | | R | TGGGTGGTCCAGGGTTTCTTACTCC |
| | | | TT |
| | | | (SEQ ID NO: 46) |
| | IL6 | F | TGGAGTCACAGAAGGAGTGGCTAAG |
| | | | (SEQ ID NO: 47) |
| | | R | TCTGACCACAGTGAGGAATGTCCAC |
| | | | (SEQ ID NO: 48) |
| | IP10 | F | ATGACGGGCCAGTGAGAATG |
| | | | (SEQ ID NO: 49) |
| | | R | GAGGCTCTCTGCTGTCCATC |
| | | | (SEQ ID NO: 50) |
| | MCP1 | F | GGCTCAGCCAGATGCAGTTAA |
| | | | (SEQ ID NO: 51) |
| | | R | CCTACTCATTGGGATCATCTTGCT |
| | | | (SEQ ID NO: 52) |

Plaque Assays and TCID50 Assays

Infectious titers of MERS-CoV and SARS-CoV-2 were determined with standard plaque assays (Chu, H. et al. (2020) Lancet Microbe Vol. 1(1), e14-e23, doi:10.1016/52666-5247(20)30004-5). Briefly, VeroE6 cells were seeded in 24-well plates 1 day before the experiment. The harvested supernatant samples were serially diluted and inoculated to the cells for 2 hours at 37° C. After inoculation, the cells were washed with PBS 3 times, and covered with 2% agarose/PBS mixed with 2×DMEM/2% FBS at 1:1 ratio. The cells were fixed after incubation at 37° C. for 72 hours. Fixed samples were stained with 0.5% crystal violet in 25% ethanol/distilled water for 10 minutes for plaque visualization. In some experiments, infectious titers of coronaviruses were determined with standard TCID50 assays. Briefly, VeroE6 cells were seeded in 96-well plates 1 day before the experiment. The harvested supernatant samples were serially diluted and inoculated to the cells for 2 hours at 37° C. After inoculation, the cells were washed with PBS 3 times and incubation at 37° C. After 72 hpi, virus titer was calculated using the Muench and Reed method.

siRNA and shRNA Knockdown

On-Targetplus caspase-6 siRNA was obtained from Dharmacon. Transfection of siRNA on MDMs was performed using Lipofectamine RNAiMAX (ThermoFisher Scientific) as described in previous studies (Chan, C. M. et al. (2016) Journal of Virology Vol. 90(20), pages 9114-9127, doi:10.1128/JVI.01133-16). Briefly, the cells were transfected with 50 nM caspase-6 siRNA for two consecutive days. At 24 hours after the second siRNA transfection, the cells were harvested in RIPA lysis buffer for Western blot analysis. In parallel, siRNA-transfected cells were challenged with MERS-CoV at 1 MOI for 1 hour at 37° C. Following the inoculation, the cells were washed with PBS and incubated for 24 hours. The virus copy number at 24 hpi was determined with RT-qPCR. pLKO.1 lentiviral caspase-6 shRNA plasmid was obtained from Dharmacon. Transfection of caspase-6 shRNA plasmid, psPAX2 packaging plasmid and pMD2.G envelope plasmid on 293T cells was performed using Lipofectamine 3000 (Thermofisher Scientific) following the manufacturer's instructions. Briefly, 293T cells in 10 cm dishes were transfected with 6 μg caspase-6 shRNA plasmid, 4.5 μg packaging plasmid, and 1.5 g envelope plasmid in FBS-supplemented DMEM medium. The supernatant was aspirated 6 hours post transfection and replaced with FBS-free medium. The following day, the supernatant containing caspase-6 shRNA lentivirus particles was harvested and was used to transduce A549 and BEAS2B cells. A549 and BEAS2B caspase-6 stable knockdowns cells were selected by 0.5 μg/mL and 0.7 μg/mL puromycin, respectively. The selected cells were challenged with MERS-CoV at 0.1 MOI for 1 hour at 37° C. The virus copy number at 1 hpi and 24 hpi was determined with RT-qPCR.

Caspase-6 Activity Assay

Huh7 cells were infected with MERS-CoV at 1 MOI for 12 hours. In parallel, Huh7 cells were stimulated with STS at 1 μM for 6 hours. Caspase-6 activity in the cell lysate was determined with the caspase-Glo-6 assay kit (Promega). The luminescence signal of caspase-6 activity was measured following the manufacturer's instructions with a multilabel plate reader Victor X3 (Perkin-Elmer).

Immunofluorescence and Histology

Immunofluorescence staining was performed as previously described with slight modifications (Chu, H. et al. (2021) Cellular and Molecular Gastroenterology and Hepatology Vol. 11(3), pages 771-781, doi: 10.1016/j.jcmgh.2020.09.017). Briefly, infected human and animal lung tissues were fixed overnight in 10% formalin. The fixed samples were then embedded in paraffin with a TP1020 Leica semi-enclosed bench top tissue processor and sectioned at 5 μm. Tissue sections were fixed, prepared on ThermoFisher Scientific Superfrost Plus slides, and allowed to dry at 37° C. overnight. Antigen retrieval was performed by heating the slides in antigen unmasking solution (Vector Laboratories) for 90 seconds. MERS-CoV and SARS-CoV-2 were detected with an in-house guinea pig anti-MERS-CoV-N immune serum and an in-house rabbit anti-SARS-CoV-2-N immune serum, respectively. Cell nuclei were labeled with the DAPI nucleic acid stain (ThermoFisher Scientific). Alexa Fluor secondary antibodies were obtained from ThermoFisher Scientific. Mounting was performed with the Diamond Prolong Antifade Mountant from ThermoFisher Scientific. Images were captured with an Olympus BX53 fluorescence microscope (Olympus Life Science, Tokyo, Japan) or a Carl Zeiss LSM 780 confocal microscope. For H&E staining, hamster lung tissue sections were stained with Gill's hematoxylin and eosin Y (ThermoFisher Scientific). The identification of the experimental conditions for H&E-stained hamster lung tissue sections were blinded and examined by a trained histopathologist. Lung pathology was graded on a scale of 0 (normal) to 4 (most severe) according to a grading system previously described (Lee, A. C. et al. (2020) Cell Rep Medicine Vol. 1(7), Article 100121, doi: 10.1016/j.xcrm.2020.100121).

Western Blot

Cells were lysed by RIPA buffer (ThermoFisher Scientific) with protease inhibitor (Roche, Basel, Switzerland). Proteins were separated with SDS-PAGE and transferred to PVDF membranes (ThermoFisher Scientific). Specific primary antibodies were incubated with the blocked membranes at 4° C. overnight, followed by horseradish peroxidase (HRP) conjugated secondary antibodies (Thermo Fisher Scientific) for 1 hour at room temperature. The signal was developed by Immobilon Crescendo Western HRP Substrate (Merck Millipore, MA, USA) and detected using automatic x-ray film processor (Advansta) or an Alliance Q9 Advanced imager (Uvitec, Cambridge, UK).

Flow Cytometry

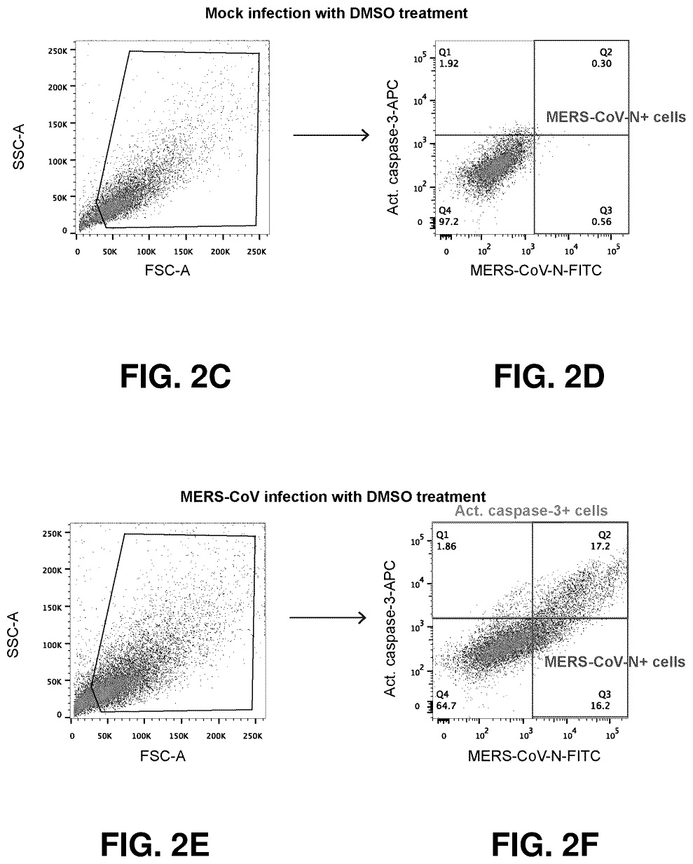

BEAS2B cells were infected with MERS-CoV at 1 MOI. At 24 hpi, the cells were detached with 10 mM EDTA in PBS, fixed in 4% paraformaldehyde, followed by immunolabeling with an in-house guinea pig anti-MERS-CoV-N immune serum and a rabbit anti-active caspase-3 antibody (BD). Flow cytometry was performed using a BD FACSCanto II flow cytometer (BD) and data was analyzed using FlowJo X 10.0.7 (BD) as previously described (Chu, H. et al. (2018) Journal Biological Chemistry Vol. 293(30), 11709-11726, doi: 10.1074/jbc.RA118.001897. The gating strategy is demonstrated in FIGS. 2A-2F.

IFN-β-Luciferase Reporter Assays

IFN-β-luciferase reporter assays were performed described previously described (Lui, P. Y. et al. (2016) Emerging Microbes and Infections Vol. 5(4), Article e39, doi: 10.1038/emi.2016.33; Siu, K. L. et al. (2014) Journal of Virology Vol. 88(9), pages 4866-4876, doi: 10.1128/JVI.03649-13). Briefly, 500 ng IFN-β-luciferase reporter plasmid, 10 ng transfection efficiency control plasmid (pNL1.1.TK, Promega), 1 g coronavirus N plasmids, 3 g caspase-6 expression plasmid, together with or without 5 g Poly(I:C) were co-transfected into 293T cells for 24 hours. On the next day, the cells were harvested for luciferase measurement with the dual-luciferase reporter assay system kit (Promega) according to the manufacturer's protocol using a multilabel plate reader Victor X3 (Perkin-Elmer). To investigate the point of action of the N fragments, IFN-β-luciferase reporter plasmid pNL1.1.TK and MERS-CoV N(1-241) or MERS-CoV N(242-413) were co-transfected into 293T cells together with expression plasmids for RIG-IN, MAVS, or TBK1. At 24 hours post transfection, the cells were harvested for luciferase measurement.

Alignment of Human, Mouse, and Hamster Caspase-6

Caspase-6 protein sequences of Homo sapiens (Uniprot ID: P55212), Mus musculus (Uniprot ID: 008738) and mesocricetus auratus (Uniprot ID: A0A1U7QNN7) were downloaded from UniProt66. Multiple sequence alignment was performed with MUSCLE (MUltiple Sequence Comparison by Log-Expectation; Edgar, R. C. (2004) Nucleic Acids Research Vol. 32(5), pages 1792-1797, doi: 10.1093/nar/gkh340). The crystal structure of caspase-6 and VEID complex was retrieved from the Protein Data Bank (PDB code: 30D5; Berman, H. M. et al. (2000) Nucleic acids research Vol. 28(1), pages 235-242, doi: 10.1093/nar/28.1.235). Caspase-6 residues within 4 Å of VEID were defined as the binding sites and visualized with Pymol.

Prediction of Potential Caspase-6 Cleavage Sites

The protein sequence of MERS-CoV N (NC_019843) was used for caspase-6 cleavage site analysis. Potential caspase-6 cleavage motifs on MERS-CoV N were determined based on published substrate specificity of caspase-629. The amino acid pattern “[TVILENYF].D” was searched against the N sequence, where “.” represented any amino acid.

Statistical Analysis

Data on figures represents means and standard deviations. Statistical comparison between different groups was performed by one-way ANOVA, two-way ANOVA, Student's t-test, or Log-rank (Mantel-Cox) test using GraphPad Prism version 9. Differences were considered statistically significant when p<0.05.

Results

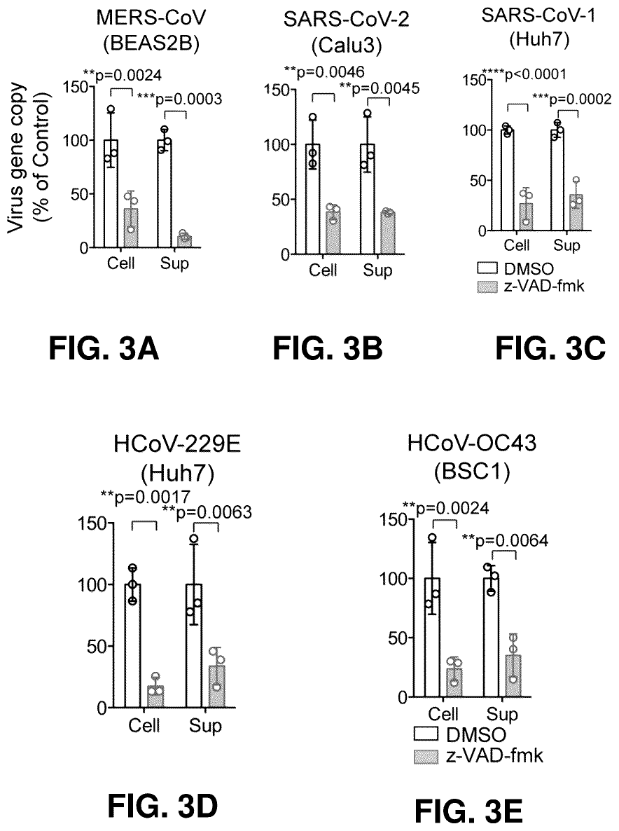

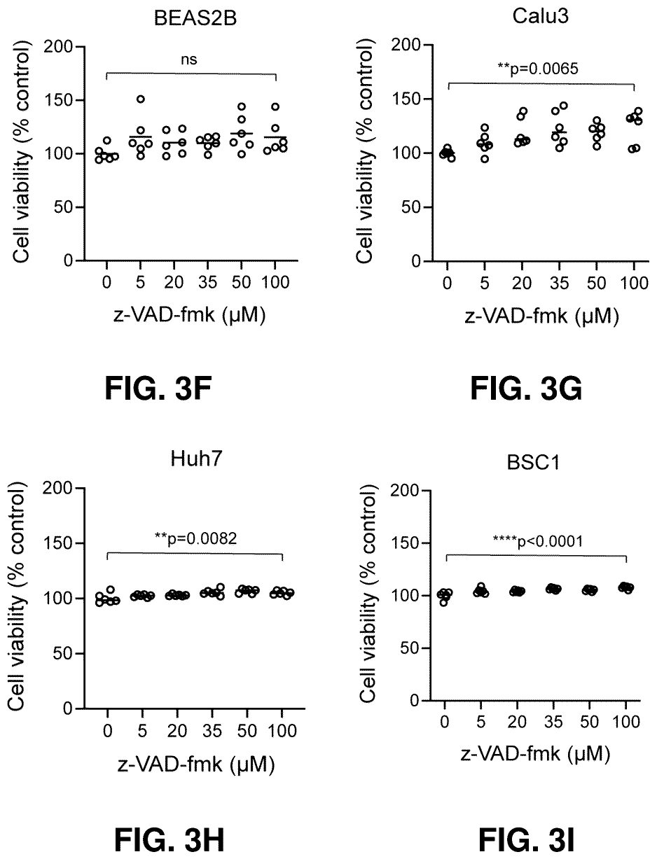

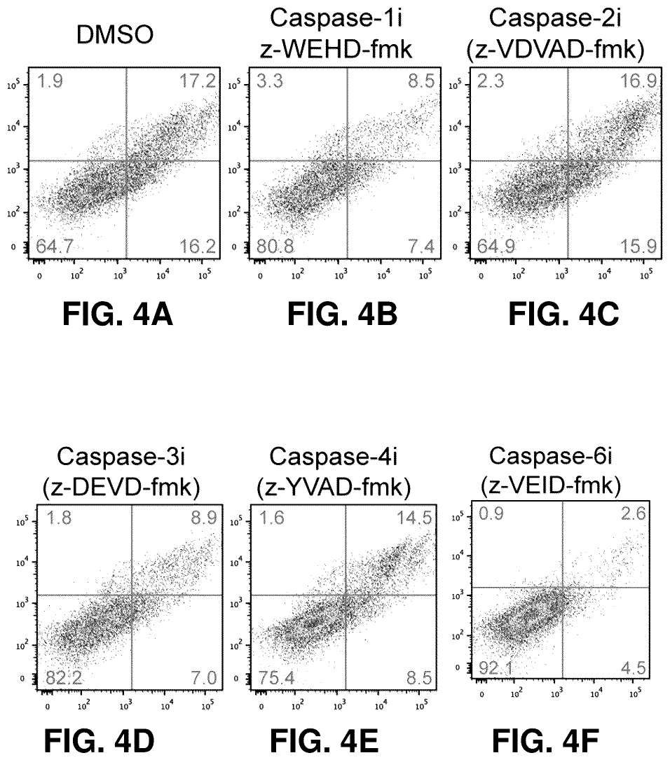

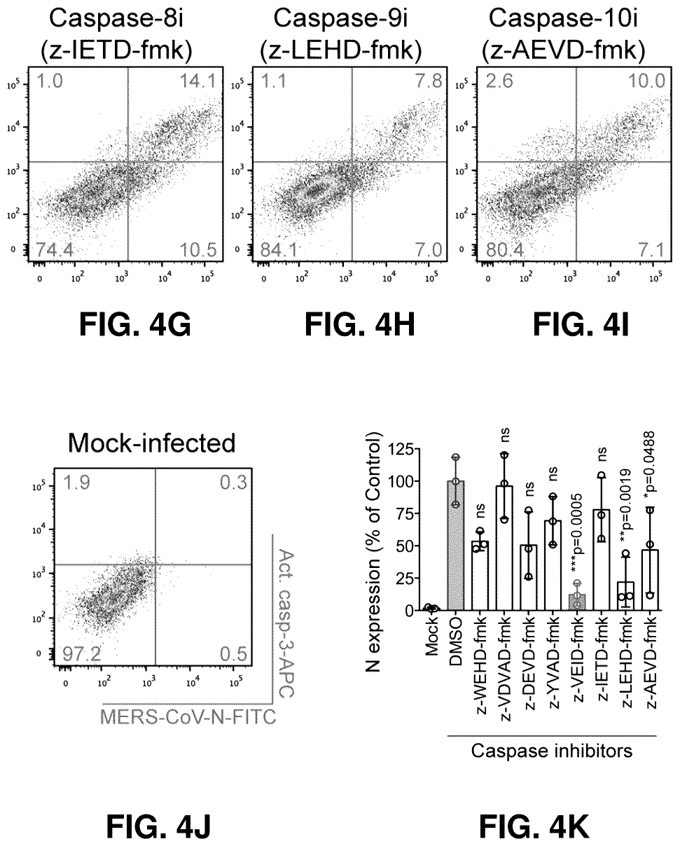

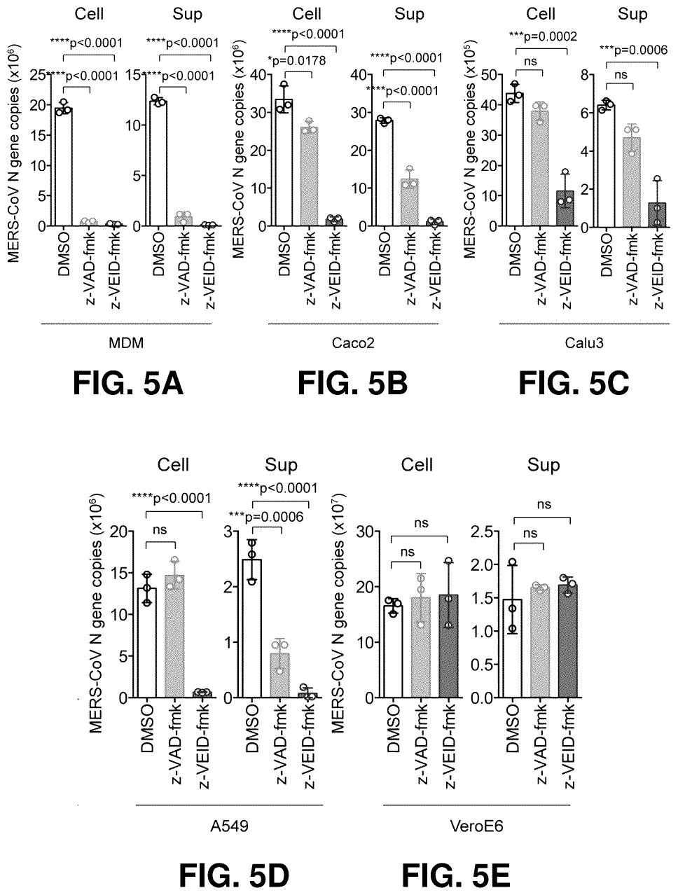

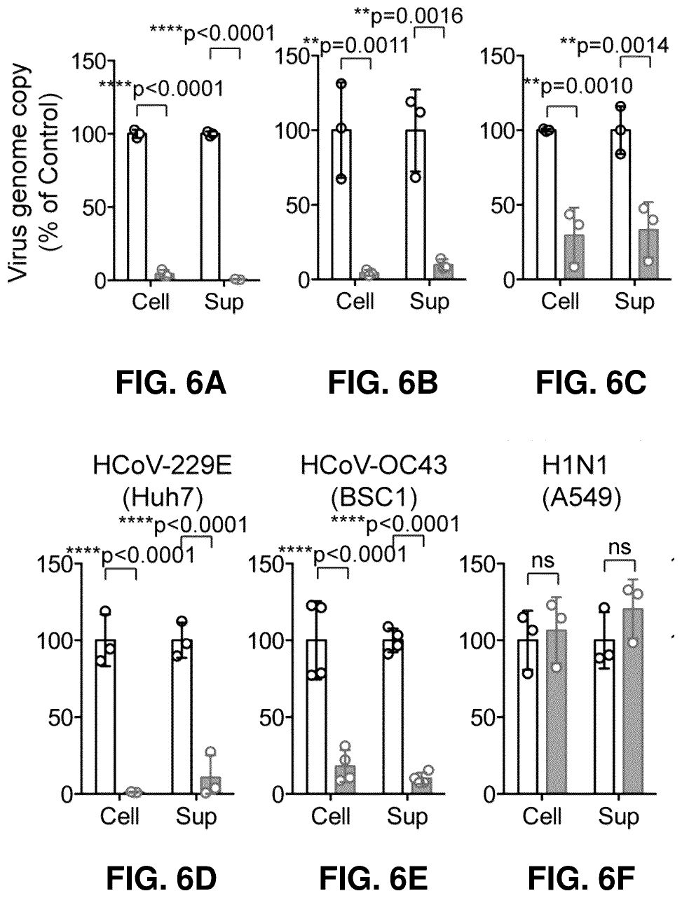

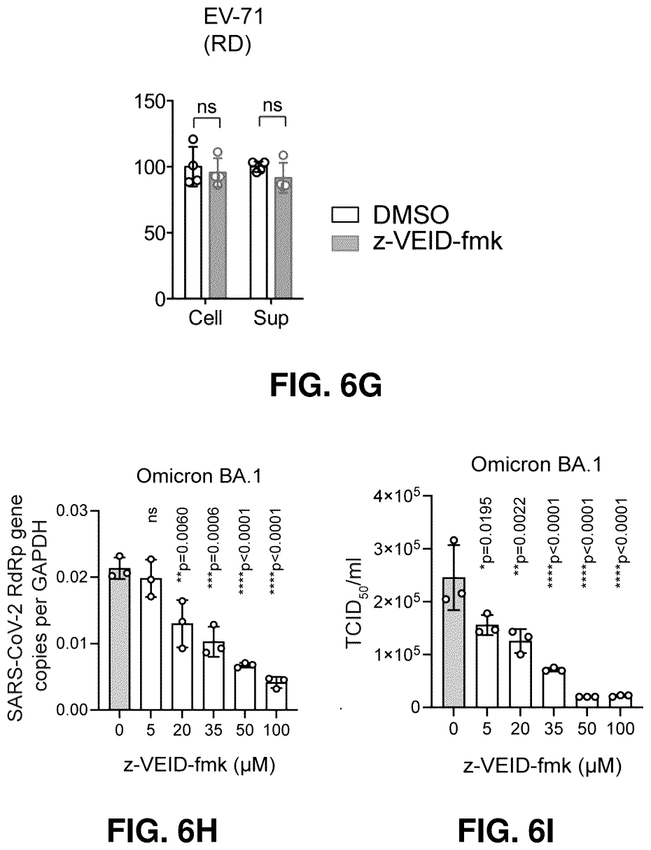

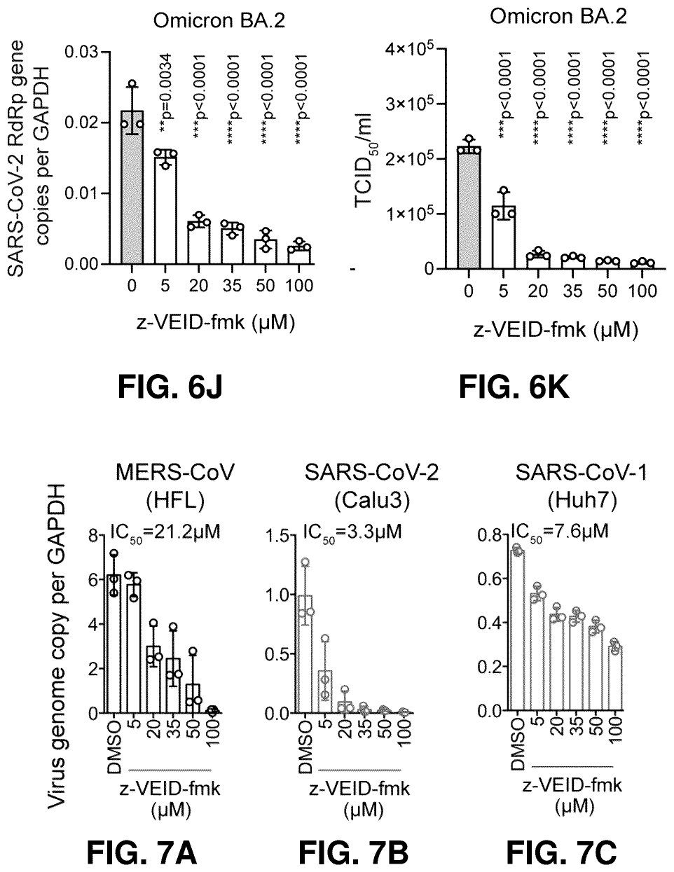

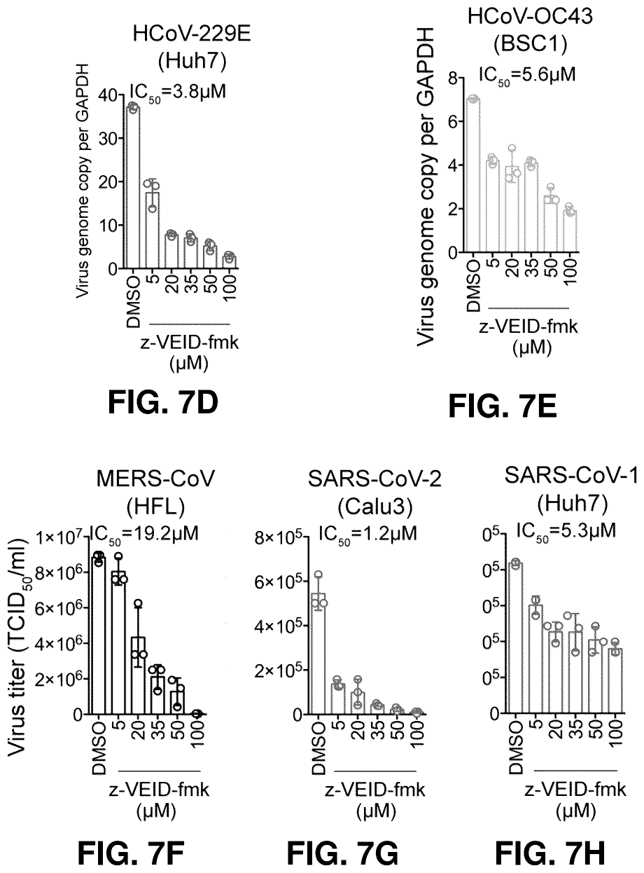

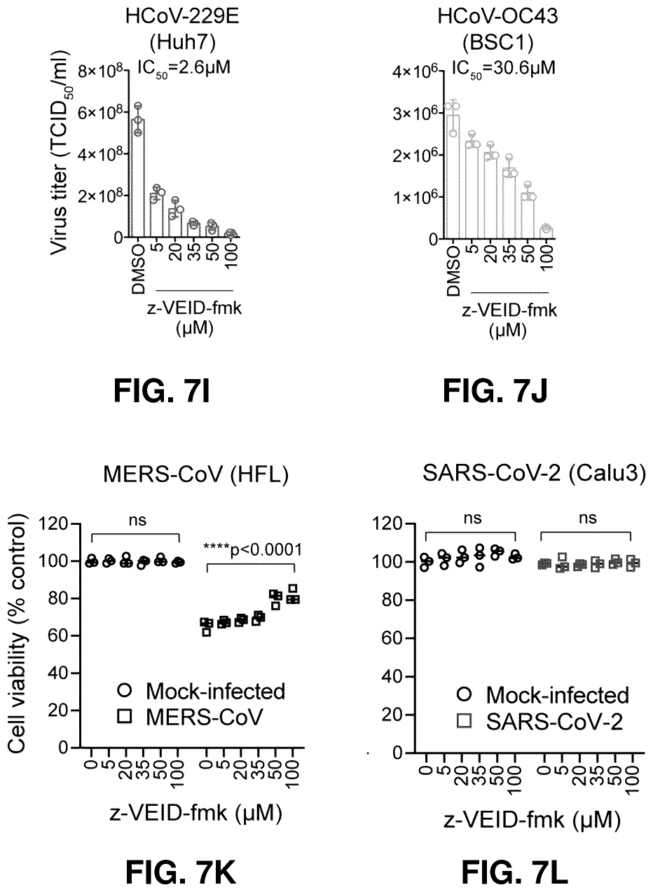

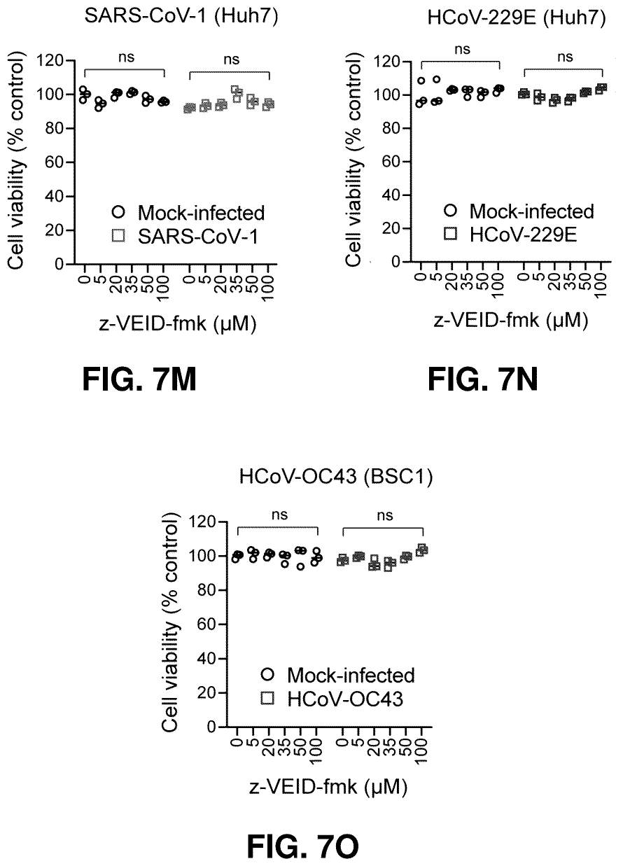

The inventors have shown that MERS-CoV infection triggers substantial apoptosis while inhibition of apoptosis with the pan-caspase inhibitor, z-VAD-fmk, significantly limited MERS-CoV replication (FIGS. 3A-3E; Yeung, M. L. et al. (2016) Nature Microbiology Vol. 1, Article 16004, doi: 10.1038/nmicrobiol.2016.4; Chu, H. et al. (2021) Science Advances Vol. 7(25), doi: 10.1126/sciadv.abf8577). Interestingly, z-VAD-fnk similarly limited the replication of other human pathogenic coronaviruses including SARS-CoV-2, SARS-CoV-1, HCoV-229E, and HCoV-OC43 (FIGS. 3A-3I), suggesting that the dependency on apoptosis or caspase activity for efficient virus replication is a conserved mechanism for coronaviruses. Caspases are cysteine-aspartic proteases that regulate the host apoptosis cascade (McIlwain, D. R., et al. (2013). Cold Spring Harbor Perspectives in Biology Vol. 5(4), Article a008656, doi: 10.1101/cshperspect.a008656). To investigate which caspase is most responsible for modulating coronavirus replication, MERS-CoV was used as a model virus and virus replication was evaluated in the presence of specific inhibitors against individual caspases. The results revealed that caspase-6 inhibition most dramatically limited MERS-CoV replication (FIGS. 4A-4K). The inhibitory effect on MERS-CoV replication by caspase-6 inhibition was conserved across different cell types exceptVeroE6 cells (FIGS. 5A-5E). Importantly, inhibition of caspase-6 with its specific inhibitor, z-VEID-fmk, attenuated the replication of all evaluated coronaviruses including that of SARS-CoV-2 and SARS-CoV-1, but did not impact the replication of influenza virus (H1N1) or enterovirus (enterovirus A71) (FIGS. 6A-6K). According to RT-qPCR and TCID50 assays, the IC50 of z-VEID-fmk against coronaviruses ranged from 3.3 μM for SARS-CoV-2 to 21.1 μM for MERS-CoV in the cell lysate samples, and 1.2 μM for SARS-CoV-2 to 30.6 μM for HCoV-OC43 in the supernatant samples, respectively (FIGS. 7A-7O).

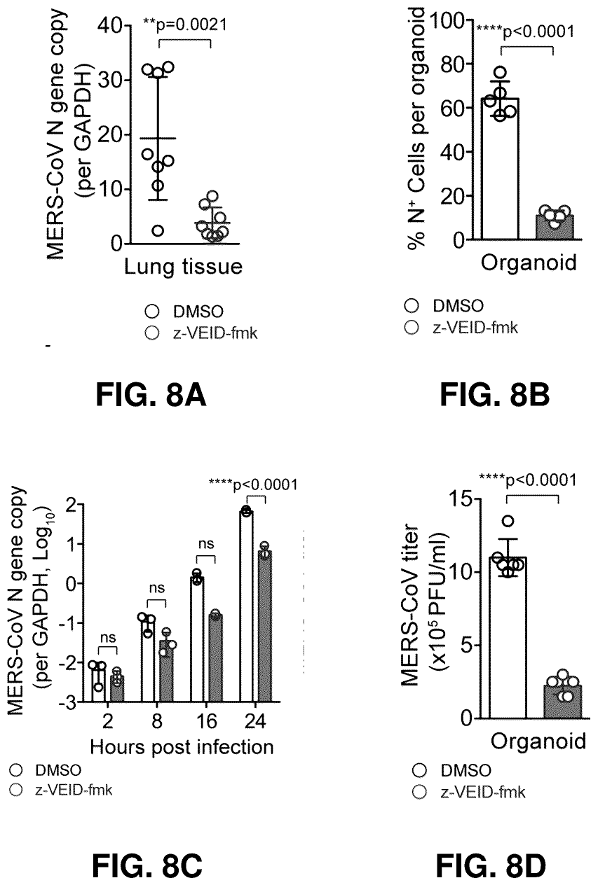

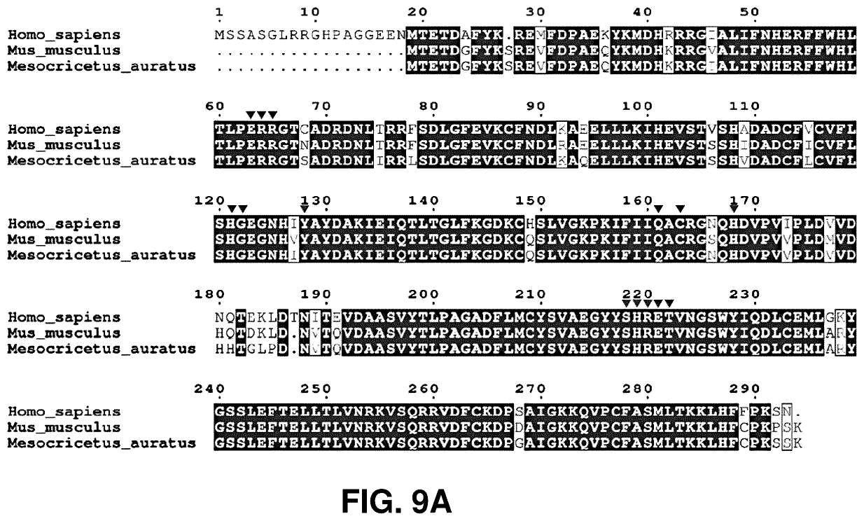

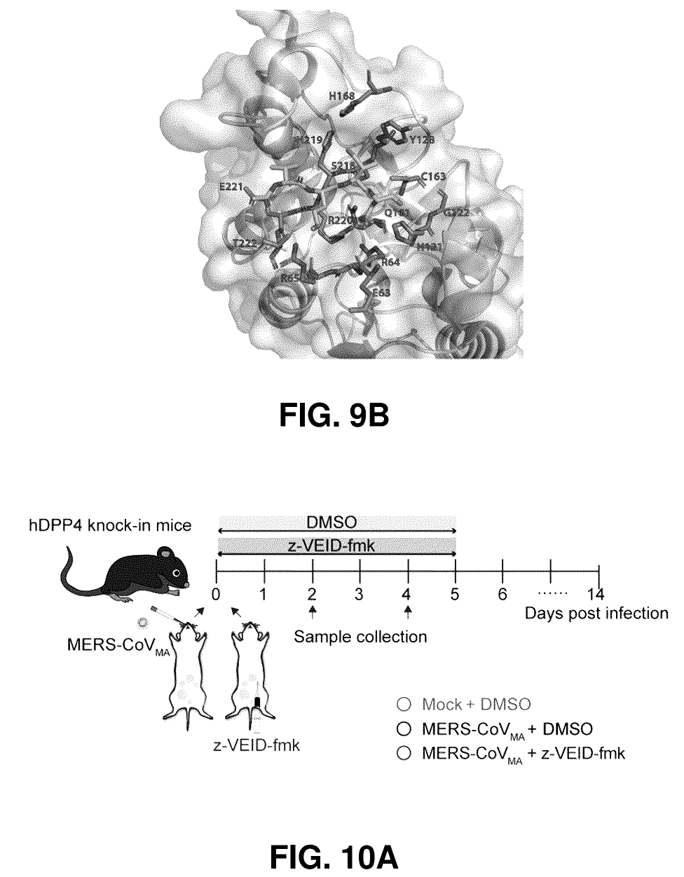

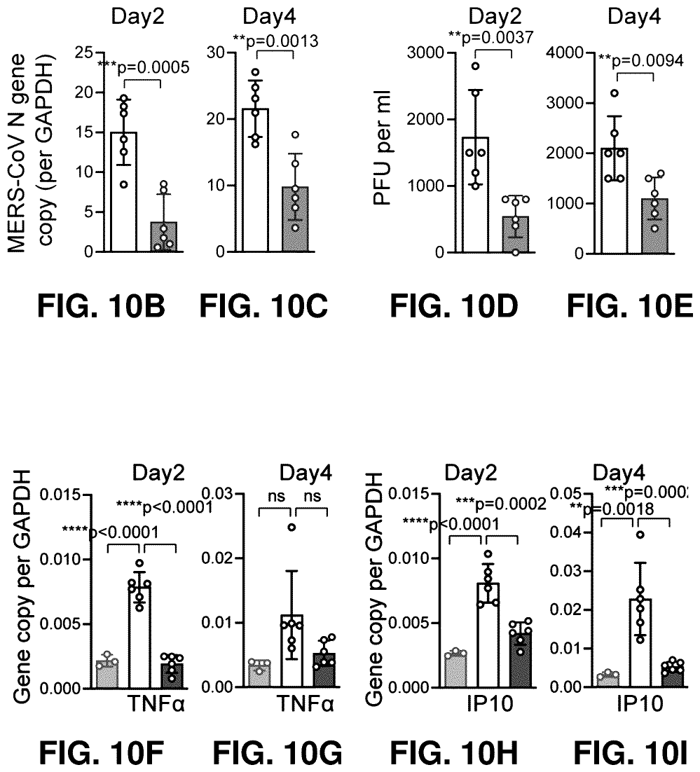

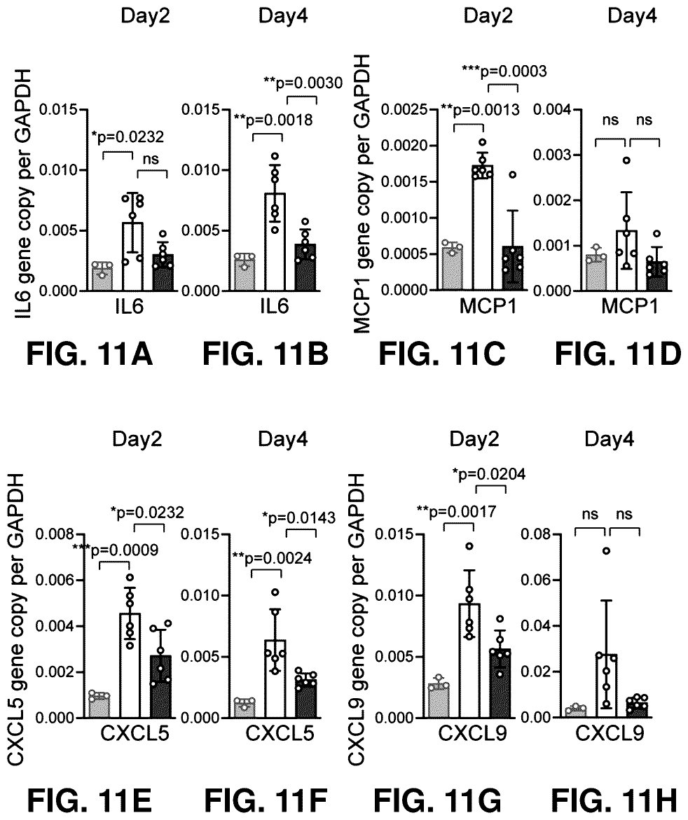

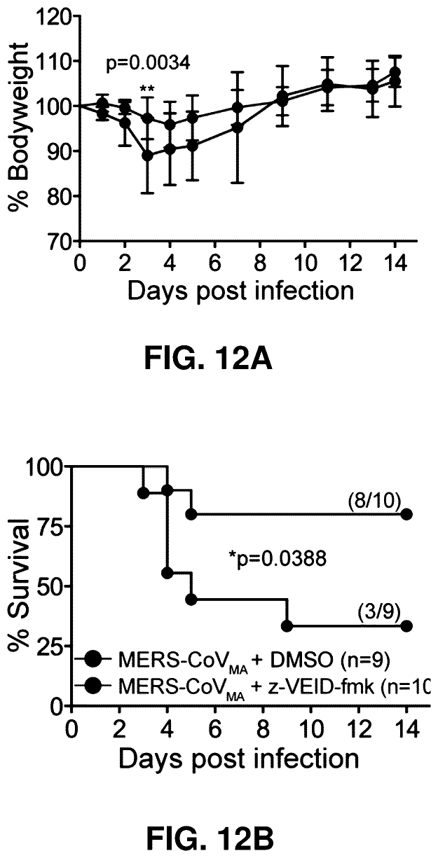

MERS-CoV and SARS-CoV-2 were used as model coronaviruses and the impact of caspase-6 inhibition on coronavirus replication in infected human ex vivo lung tissues, human intestinal organoids, and animals was evaluated. In human lung tissues, caspase-6 inhibition with z-VEID-fnk significantly reduced MERS-CoV nucleocapsid (N) protein and N gene expression (FIG. 8A). Similarly, z-VEID-fmk inhibited MERS-CoV replication in human intestinal organoids and inhibited the production of infectious virus particles by approximately 80% (p<0.0001) at 24 hours post infection (hpi) (FIGS. 8B-8D). Caspase-6 is largely conserved among mammals and the z-VEID-fmk binding pocket is conserved among humans, mice, and hamsters, allowing evaluation of the effect of caspase-6 inhibition with z-VEID-fmk in these animal models (FIG. 9A). To evaluate the impact of caspase-6 inhibition on MERS-CoV replication in vivo, human DPP4 knock-in (hDPP4 KI) mice were infected with mouse adapted MERS-CoV (MERS-CoV MA; Li, K. et al. (2017) Proceedings of the National Academy of Sciences of the U.S.A. Vol. 114(15), pages E3119-E3128, doi: 10.1073/pnas.1619109114) and then treated the mice with z-VEID-fmk or DMSO (FIG. 10A). A subset of the mice was harvested at day 2 and day 4 post infection. The mouse lungs were immunolabelled to detect MERS-CoV N expression. The results demonstrated that z-VEID-fmk effectively reduced MERS-CoV MA replication in the lungs of the infected mice at both day 2 and day 4 post infection (FIGS. 10B-10E), and significantly attenuated the expression of pro-inflammatory cytokines and chemokines (FIGS. 10F-10I and FIGS. 11A-11H). Importantly, the z-VEID-fmk treatment largely inhibited body weight loss and significantly improved the survival of the infected hDPP4 KI mice from 33.3% to 80% (3/9 vs 8/10; p=0.0388) (FIGS. 12A and 12B).

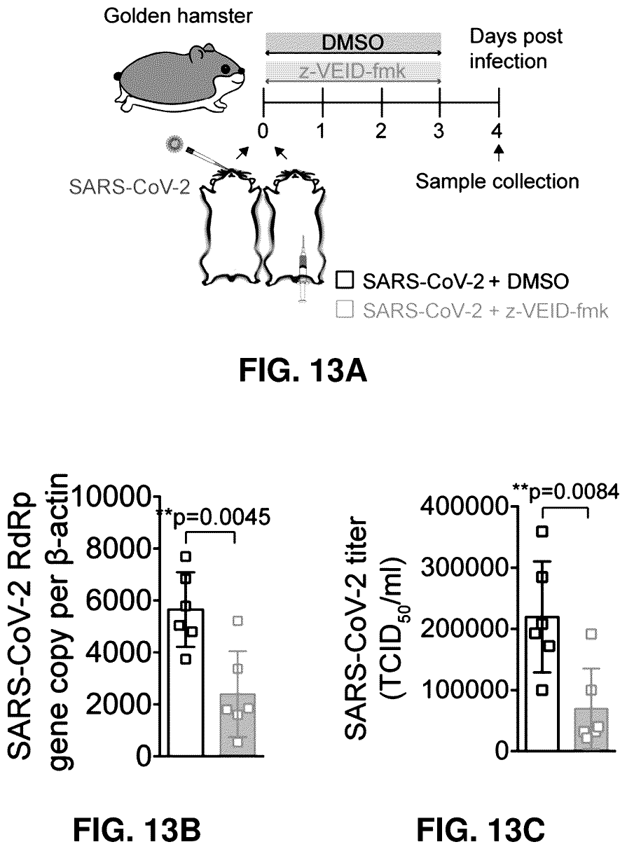

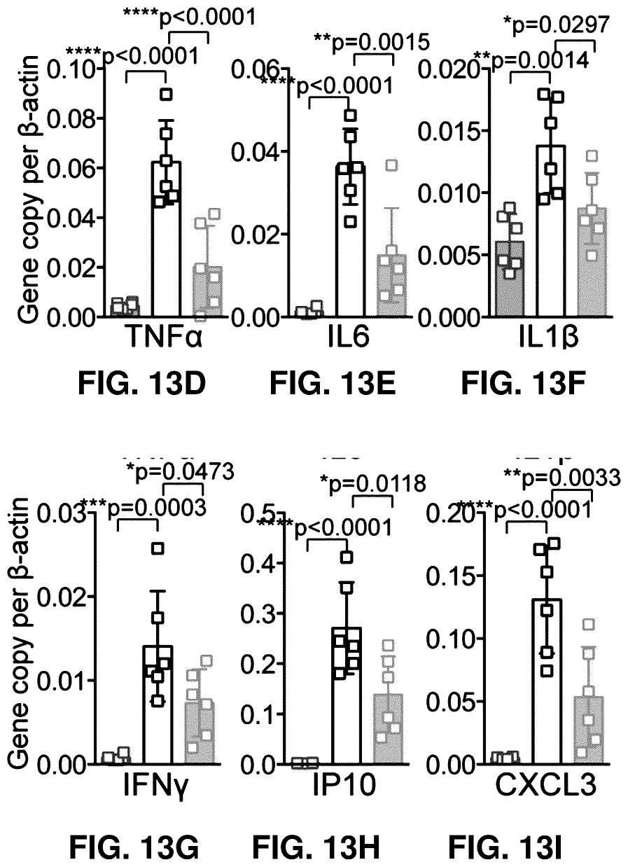

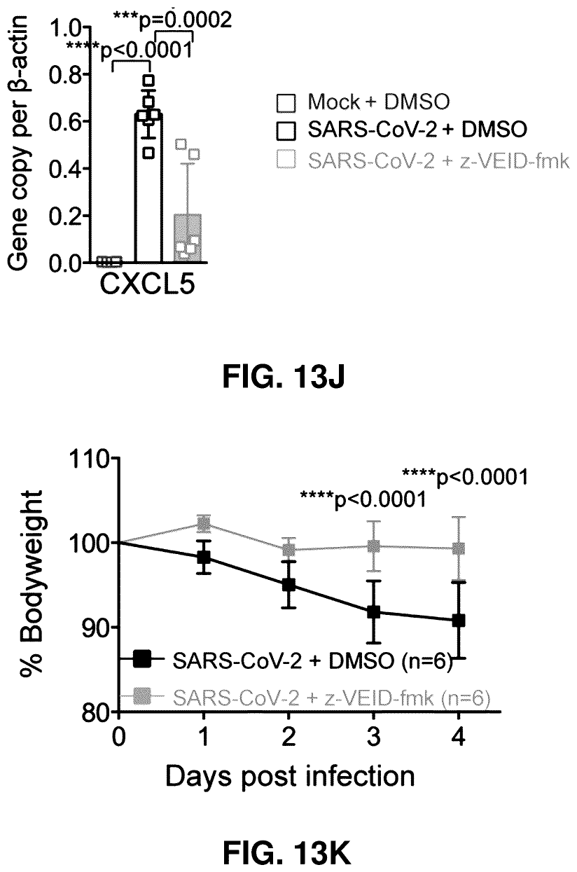

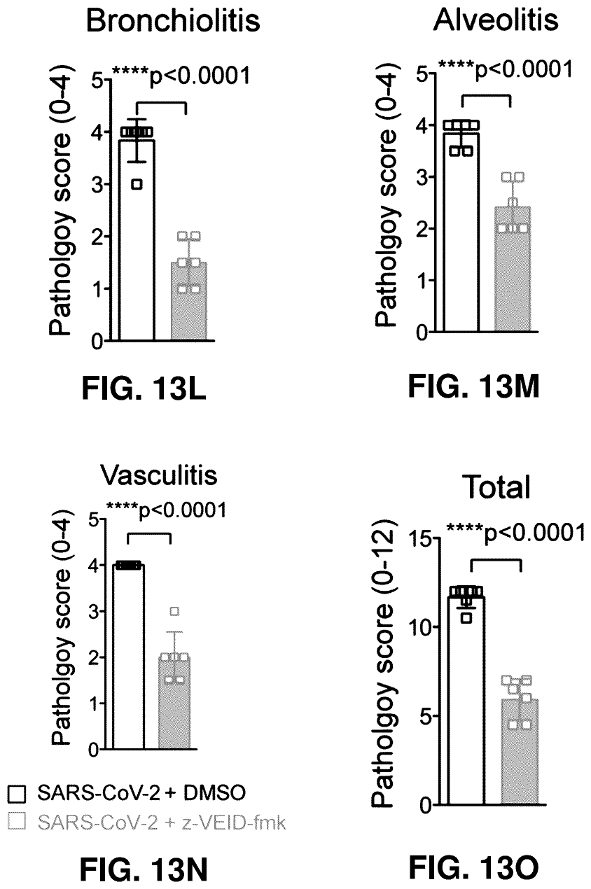

It was then explored whether z-VEID-fmk could similarly inhibit SARS-CoV-2 replication in vivo. To this end, golden hamsters were infected with SARS-CoV-2 through the intranasal route and treated the hamsters with z-VEID-fmk or DMSO (FIG. 13A). The results demonstrated that caspase-6 inhibition with z-VEID-fmk significantly reduced SARS-CoV-2 replication in the hamster lungs (FIGS. 13B and 13C) including small airways and alveoli. Viral N protein expression in the small airways and alveoli of infected hamster lungs with or without z-VEID-fmk treatment was revealed with immunofluorescence staining with the in-house rabbit immune serum against SARS-CoV-2 N. The results revealed that caspase-6 inhibition reduced the number of viral N-protein positive cells and ameliorated the lung pathology of SARS-CoV-2-infected golden Syrian hamsters. Also, caspase-6 inhibition also ameliorated the expression of virus-induced pro-inflammatory cytokines and chemokines (FIGS. 13D-13J). The attenuated virus replication and expression of pro-inflammatory markers resulted in significant improvements of the body weight of the infected hamsters (FIG. 13K). In addition, the lung tissues were harvested from the hamsters at day 4 post infection to evaluate histological changes. Haematoxylin and eosin (H&E) staining was performed on the hamster lungs. Pulmonary blood vessel wall inflammation and endothelium infiltration were frequently observed. Mock-infected hamster lung sections demonstrated normal histology showing (i) intact bronchiolar epithelium lining, (ii) thin alveolar wall and clear air sac, and (iii) a normal structure of pulmonary blood vessel section. In SARS-CoV-2-infected hamsters, lung tissues had diffused inflammatory infiltration and exudation with disappearing air-exchange structures. The characteristic histopathological changes included (i) peribronchiolar infiltration and bronchiolar epithelium desquamation, (ii) alveolar infiltration and haemorrhage with alveolar space filled with infiltrated immune cell and protein-rich exudate, and (iii) pulmonary blood vessel showed immune cells infiltration in the vessel wall, endothelium, and perivascular connective tissue. The hamster lung pathology was markedly improved with z-VEID-fmk treatment demonstrating (i) a milder degree of immune cell infiltration in bronchiolar epithelium and peribronchiolar tissue, (ii) thickened alveolar wall with red blood cells but alveolar space showing no immune cell infiltration nor exudation, and (iii) pulmonary vessel wall showing a few immune cells attached to the endothelium.

Infected lungs from hamsters in the mock treatment group showed severe bronchiolar epithelial cell death and desquamation, extensive alveolar space mononuclear cell infiltration, protein rich fluid exudation, alveolar hemorrhage, and severe destruction of alveolar structure. These histopathological changes were consistent with inventors' previous studies in SARS-CoV-2-infected golden hamsters (Chan, J. F. et al. (2020) Clinical Infectious Diseases Vol. 71(9), pages 2428-2446, doi: 10.1093/cid/ciaa325).

In contrast, in the lungs of z-VEID-fmk-treated hamsters, all categories of tissue damage, including bronchiolitis, alveolitis and vasculitis, were significantly ameliorated. In these animals, a mild degree of bronchiolar epithelium desquamation, focal alveolar septal congestion, and mild perivascular infiltration were observed. At the same time, the z-VEID-fmk treatment dramatically inhibited alveolar space immune cells infiltration, albeit some degree of hemorrhaging was still visible in the treatment group. To quantitatively evaluate the severity of lung damage, semi-quantitative histopathological examination of the bronchioles, alveoli, and blood vessels using inventors' previously described methods were performed (Lee, A. C. et al. (2020) Cell Reports Medicine Vol. 1(7), Article 100121, doi: 10.1016/j.xcrm.2020.100121). The histopathological scores demonstrated that the z-VEID-fmk treatment significantly ameliorated lung damage in the infected hamsters (FIGS. 13L-13O). Taken together, these results demonstrates that caspase-6 inhibition attenuates coronavirus replication in cell culture, human lung tissue, organoid, and animal settings.

### Example 2: Caspase-6 Cleaves Nucleocapsid (N) Protein and Modulates Coronavirus Replication at a Post-Entry Step

Materials and Methods

The materials and methods are the same as described for Example 1.

Results

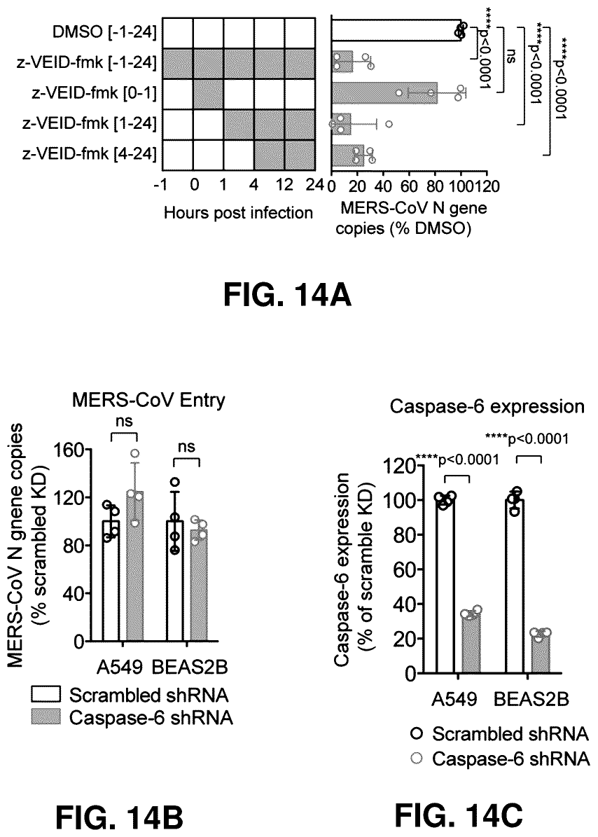

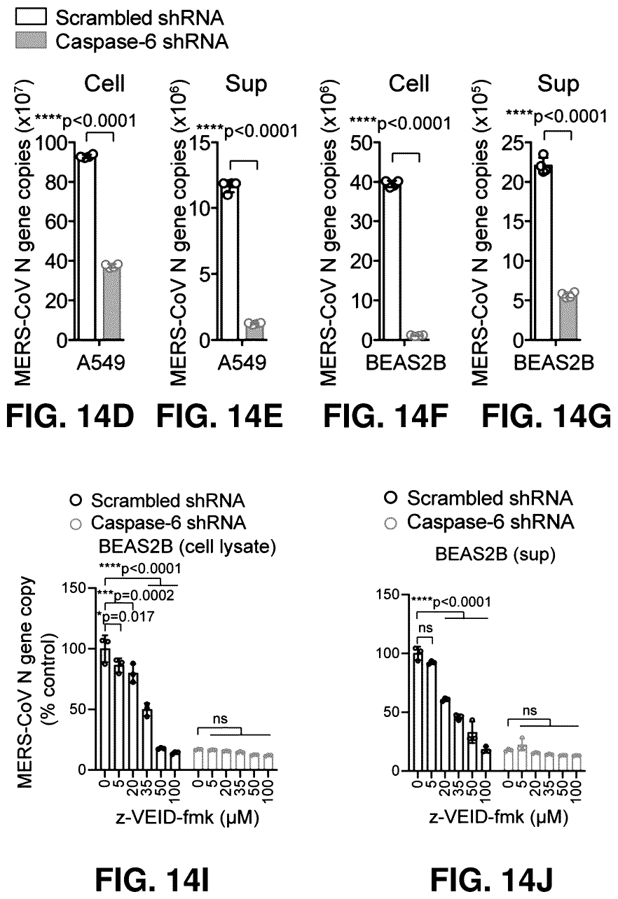

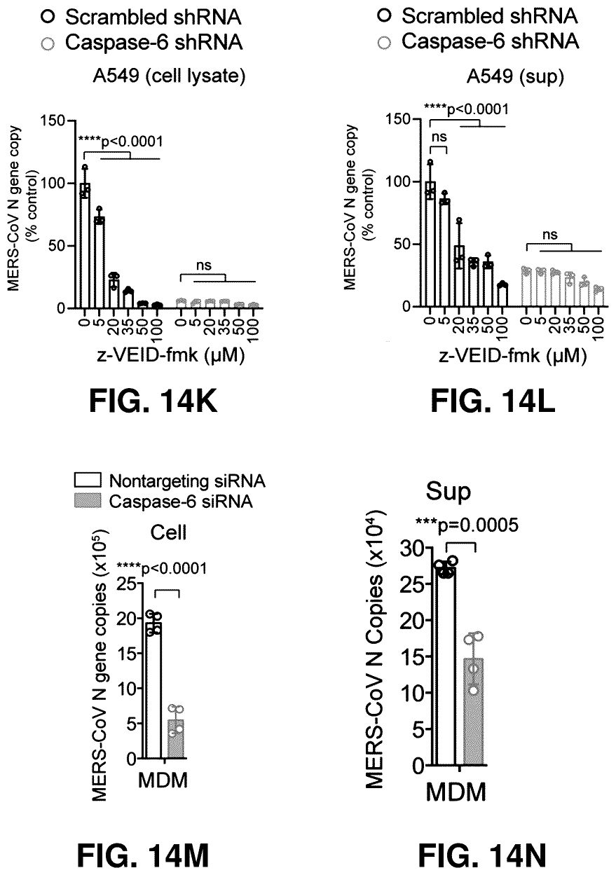

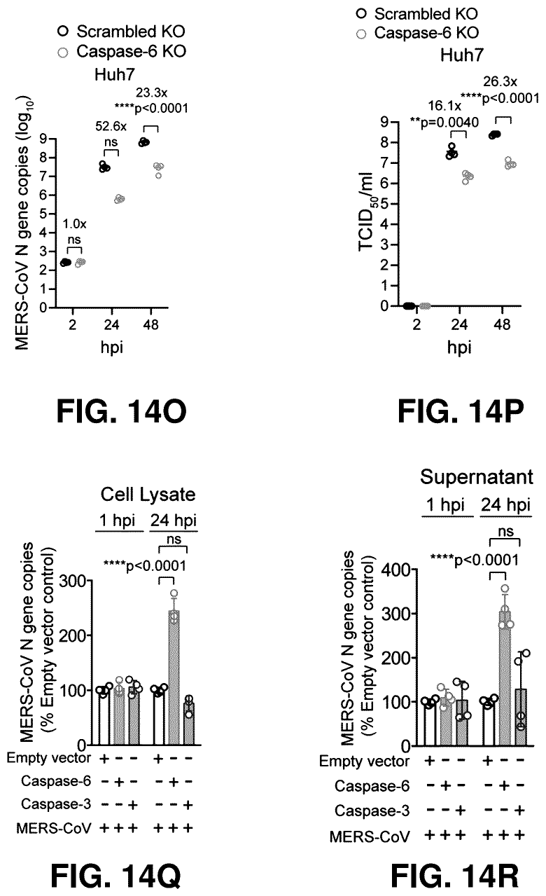

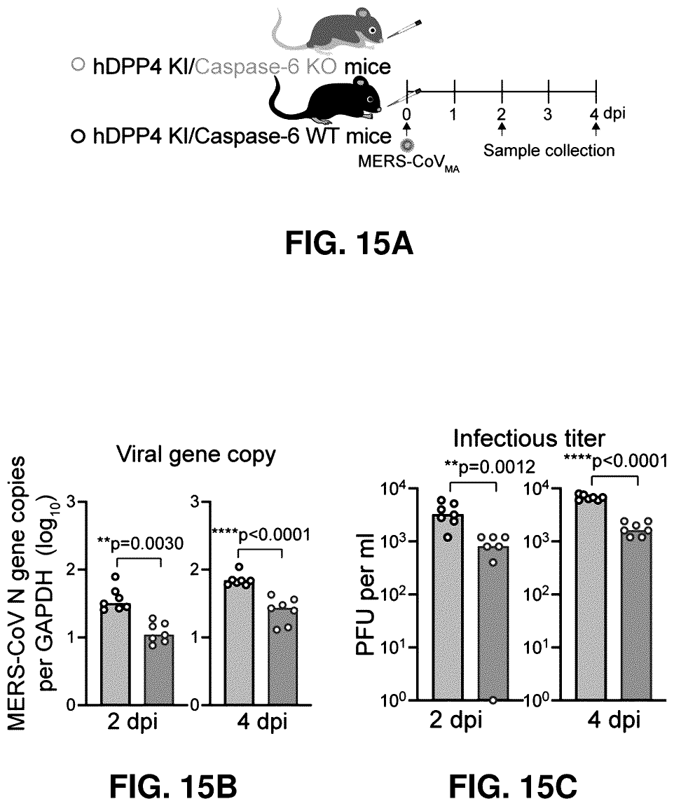

The role of caspase-6 in coronavirus replication was explored. Using MERS-CoV as a model, the effect of z-VEID-fmk was assessed using a time-of-addition assay. The results showed that z-VEID-fmk added during MERS-CoV inoculation did not reduce virus replication (FIG. 14A). Consistent with this finding, the entry of MERS-CoV in caspase-6-stable knockdown A549 and BEAS2B cells was not compromised (FIGS. 14B and 14C), confirming the notion that caspase-6 did not play a role in MERS-CoV entry. Next, MERS-CoV N gene expression was evaluated in caspase-6 stable knockdown A549 and BEAS2B cells harvested at 24 hpi. Among these samples, MERS-CoV replication was significantly reduced in the presence of caspase-6 knockdown (FIGS. 14D-14G). The stable scrambled knockdown or caspase-6 knockdown A549 and BEAS2B cells were treated with the caspase-6 inhibitor, z-VEID-fmk. The results showed that the viral inhibitory effect of z-VEID-fnk was largely diminished among the stable caspase-6 knockdown A549 and BEAS2B cells (FIGS. 14I-14L). The role of caspase-6 on MERS-CoV replication was further investigated in human primary monocyte-derived macrophages (MDMs). Caspase-6 protein expression was measured from caspase-6 siRNA transfected MDMs. In these cells, transient depletion of caspase-6 with siRNA markedly reduced MERS-CoV replication in both cell lysates and supernatant samples (FIGS. 14M and 14N). For a more complete depletion of caspase-6, caspase-6 CRISPR knockout (KO) was performed in Huh7 cells. The results demonstrated that MERS-CoV replication is significantly compromised in the caspase-6 KO Huh7 cells in comparison to the caspase-6 intact cell controls (FIGS. 14O and 14P). In addition to the gene depletion studies, MERS-CoV replication was tested in caspase-6-overexpressed cells. This was done by infecting Caspase-6- or caspase-3-overexpressed 293T cells with MERS-CoV at 1 MOI. MERS-CoV replication was quantified at 1 hpi and 24 hpi with RT-qPCR against the MERS-CoV N gene. The results demonstrated that caspase-6 but not caspase-3 overexpression efficiently promoted MERS-CoV replication (FIGS. 14Q and 14R). The caspase-6 inhibitor (z-VEID-fmk) may have cross-reactivity against other caspases (Groborz, K. et al. (2020) Cell Death Differentiation Vol. 27(2), pages 451-465, doi:10.1038/s41418-019-0364-z; McStay, G. P. et al. (2008) Cell Death Differentiation Vol. 15(2), pages 322-331, doi: 10.1038/sj.cdd.4402260). To further validate the in vivo importance of caspase-6 on coronavirus replication, caspase-6 KO was performed in the background of hDPP4 KI mice (FIG. 1A). Knock-out of caspase-6 expression was confirmed via western blot analysis. The mice were challenged with MERS-CoV MA (FIG. 15A). hDPP4 KI/caspase-6 KO and hDPP4 KI/caspase-6 WT mice were intranasally inoculated with 2.5×10³ PFU MERS-CoV MA. Mouse lung samples were harvested at day 2 and day 4 post infection and stained with haematoxylin and eosin (FIG. 15A). The results demonstrated that MERS-CoV MA replication and MERS-CoV MA-induced lung damage were significantly reduced in the lungs of hDPP4 KI/caspase-6 KO mice in comparison to that in the hDPP4 KI/caspase-6 WT mice (FIG. 15B and FIG. 15C). Collectively, these results indicate that caspase-6 is required for efficient MERS-CoV replication in vitro and in vivo.

### Example 3: Caspase-6-Mediated N Cleavage Modulates Interferon Response and is Conserved Across Human-Pathogenic Coronaviruses

Materials and Methods

The materials and methods are the same as described for Example 1.

Results

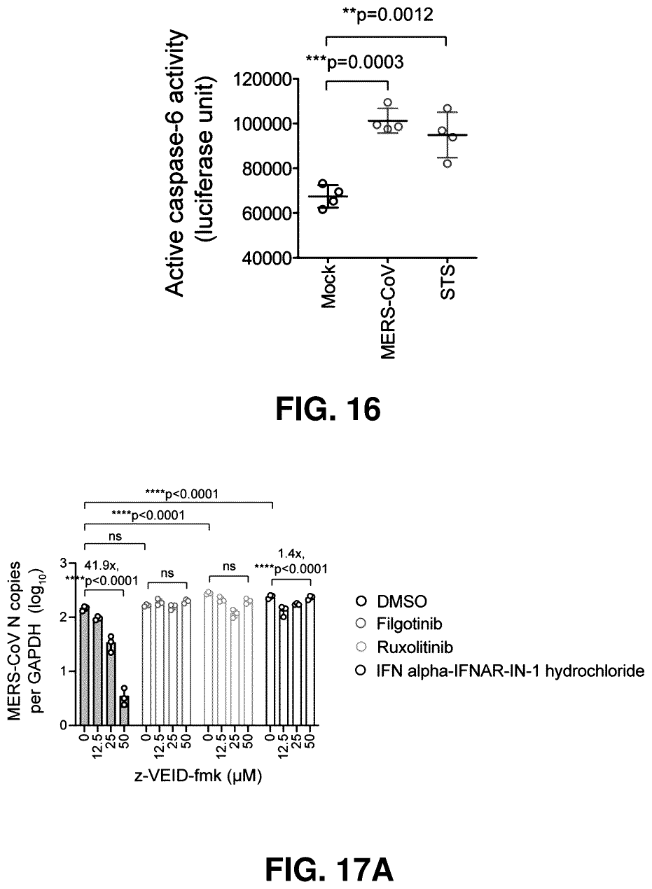

Together with caspase-3 and caspase-7, caspase-6 is one of the three executor caspases that execute apoptosis by proteolytic cleavage of host substrates (Parrish, A. B. et al. (2013) Cold Spring Harbor Perspectives in Biology Vol. 5(6), Article a008672, doi: 10.1101/cshperspect.a008672). Caspase-6 is cleavage activated when apoptosis is induced but can also undergo autoactivation (FIG. 16; Wang, X. J. et al. (2015) Annual Reviews in Pharmacology and Toxicology Vol. 55, pages 553-572, doi: 10.1146/annurev-pharmtox-010814-124414). As a cysteine-aspartic protease, it is hypothesized that caspase-6 may modulate coronavirus replication by acting on a viral component. To this end, caspase-6 was co-expressed with various MERS-CoV components and viral protein cleavage by caspase-6 was evaluated when apoptosis was induced. Cells were harvested for Western blot at 24 hours post transfection. In these assays, 1 μM staurosporine (STS) was used to trigger apoptosis to mimic the apoptotic environment in MERS-CoV-infected cells at 6 hours before sample harvest. The results demonstrated that caspase-6 mediated the cleavage of the viral N protein but not other viral components. Cleavage of N was completely abolished in the presence of the specific caspase-6 inhibitor, z-VEID-fmk, suggesting that the cleavage is caspase-6 specific. Importantly, N cleavage was readily detected in cells infected by MERS-CoV and was similarly inhibited by caspase-6 inhibition in the infected cells. In addition, N was only cleaved by capsase-6, but not by the key executor caspase, caspase-3. Together, these results demonstrate that caspase-6 specifically cleaves MERS-CoV N.

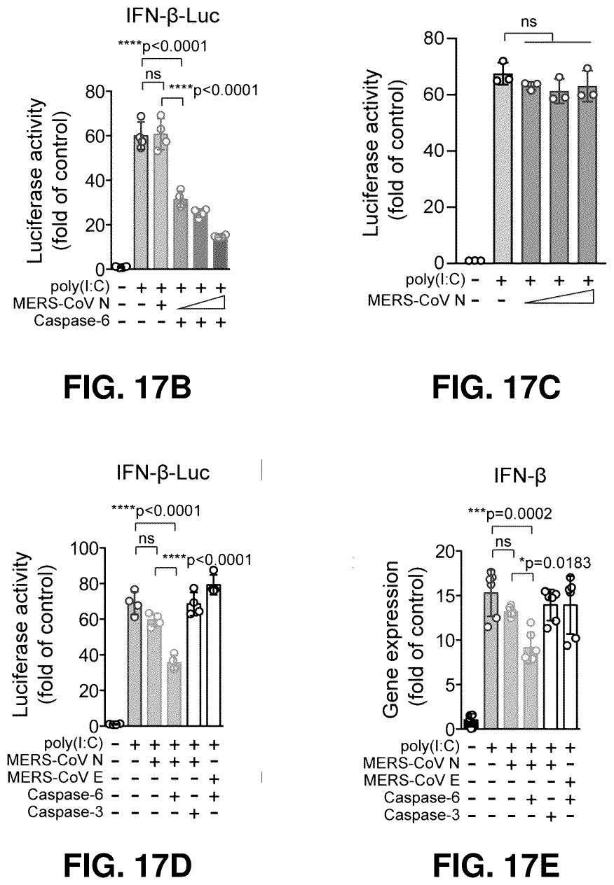

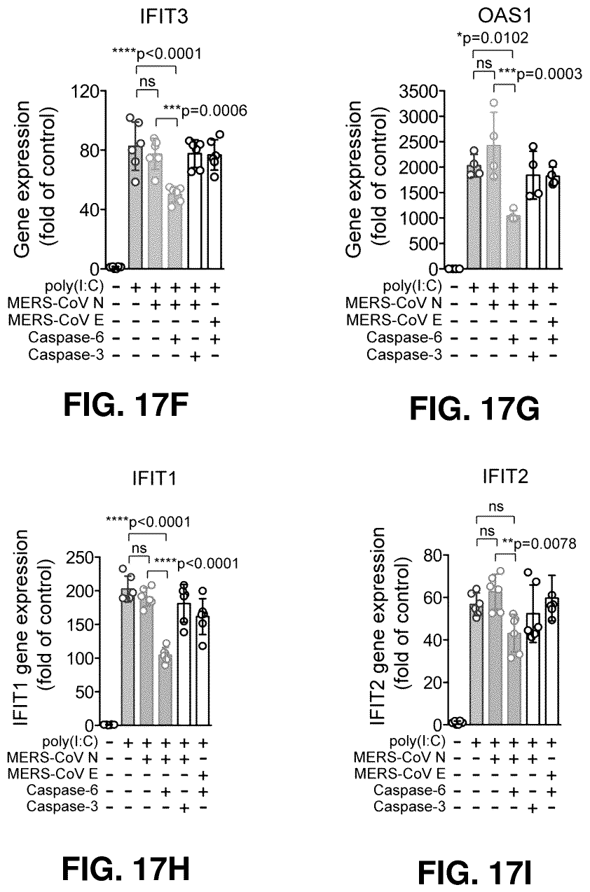

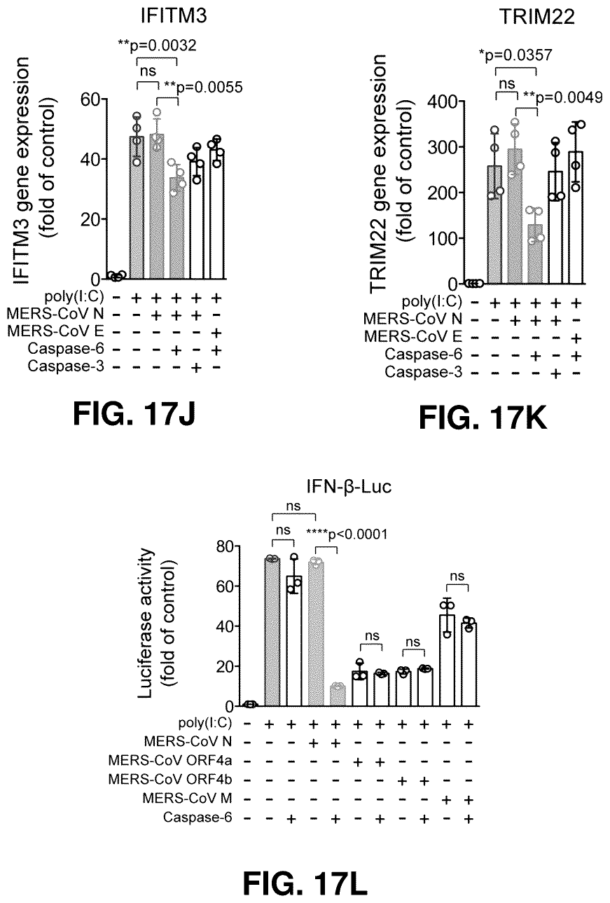

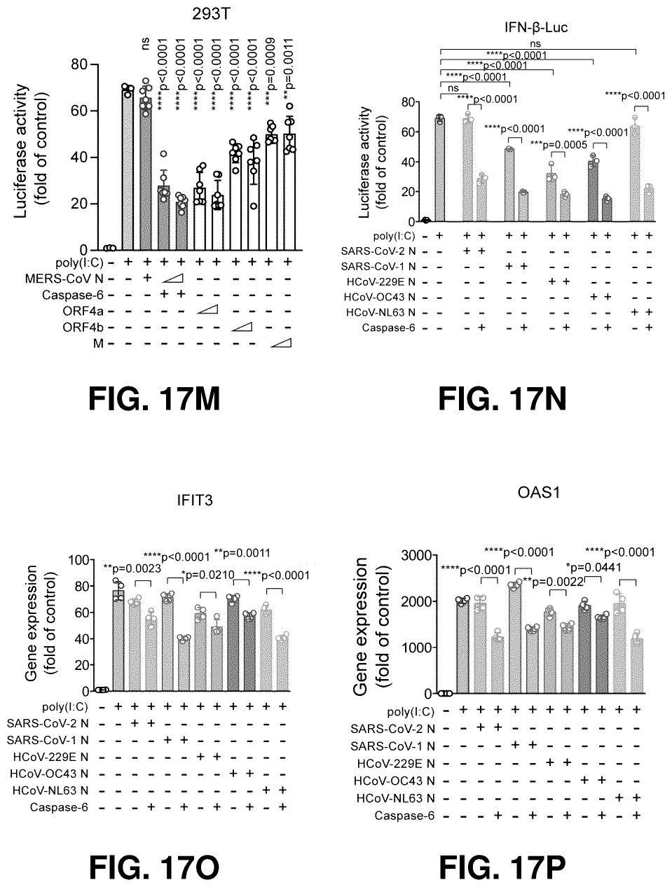

Next, how caspase-6-mediated N cleavage modulates coronavirus replication was explored. It was demonstrated in Example 1 that caspase-6 inhibition attenuated MERS-CoV replication in all evaluated cell types except for VeroE6 (FIGS. 5A-5E). VeroE6 cells are deficient in interferon (IFN) signaling due to a homozygous deletion in the type-I IFN gene cluster (Desmyter, J. et al. (1968). Journal of Virology Vol. 2(10), pages 955-961, doi: 10.1128/JVI.2.10.955-961.1968; Osada, N. et al. (2014) DNA Research 21(6), pages 673-683, doi: 10.1093/dnares/dsu029). MERS-CoV-infected Huh7 cells was treated with z-VEID-fmk in the presence of DMSO, Filgotinib (JAK inhibitor), Ruxolitinib (JAK inhibitor), or IFN alpha-IFNAR-IN-1 hydrochloride (IFNAR inhibitor). The results demonstrated that the antiviral effect of z-VEID-fmk diminished when the IFN pathway is non-functional by JAK or IFNAR inhibition (FIG. 17A). These results hinted that caspase-6-mediated N cleavage might modulate coronavirus replication by regulating IFN signaling. To test this hypothesis, the role of caspase-6 and MERS-CoV N in regulating IFN response was evaluated with IFN-β-reporter assays. Interestingly, the results demonstrated that MERS-CoV N co-expressed with caspase-6 suppressed IFN-β-reporter activation in a dose-dependent manner (FIGS. 17B and 17C). In parallel, co-expression of caspase-3 and MERS-CoV N or caspase-6 and MERS-CoV envelope (E) protein did not significantly impact IFN-β-reporter activation (FIG. 17D). In addition to IFN-β-reporter activity, co-expression of caspase-6 and MERS-CoV N similarly reduced the expression of IFN-β and representative interferon-stimulated genes (ISGs) including IFIT1, IFIT2, IFIT3, IFITM3, TRIM22, and OAS1 (FIGS. 17E-17G and FIGS. 17H-17K). Recent studies have reported MERS-CoV ORF4a, ORF4b, and membrane (M) protein as potent IFN antagonists (Lui, P. Y. et al. (2016) Emerging Microbes and Infections Vol. 5(4), Article e39, doi:10.1038/emi.2016.33; Siu, K. L. et al. (2014) Journal of Virology 88(9), pages 4866-4876, doi: 10.1128/JVI.03649-13; Yang, Y. et al. (2013) Protein Cell 4(12), pages 951-961, doi: 10.1007/s13238-013-3096-8). The data revealed that caspase-6 did not modulate the IFN antagonism of these known IFN antagonists (FIG. 17L) and caspase-6-cleaved N modulated the IFN response at comparable or better levels than these known IFN antagonists (FIG. 17M). Importantly, further investigations demonstrated that caspase-6 similarly mediated N cleavage of other human pathogenic coronaviruses, including that of SARS-CoV-2 and SARS-CoV-1 which agrees with findings described in Example 1 indicating that caspase-6 inhibition attenuated the replication of these coronaviruses (FIGS. 6A-6G and 7A-7J). The N genes of these coronaviruses were then expressed together with caspase-6, which revealed that the co-expression of coronavirus N with caspase-6 antagonized IFN-β-reporter activation and reduced the expression of representative ISGs including IFIT3 and OAS1 (FIGS. 17N-17P). Collectively, these results suggest that caspase-6-mediated N cleavage modulates coronavirus replication by regulating IFN signaling.

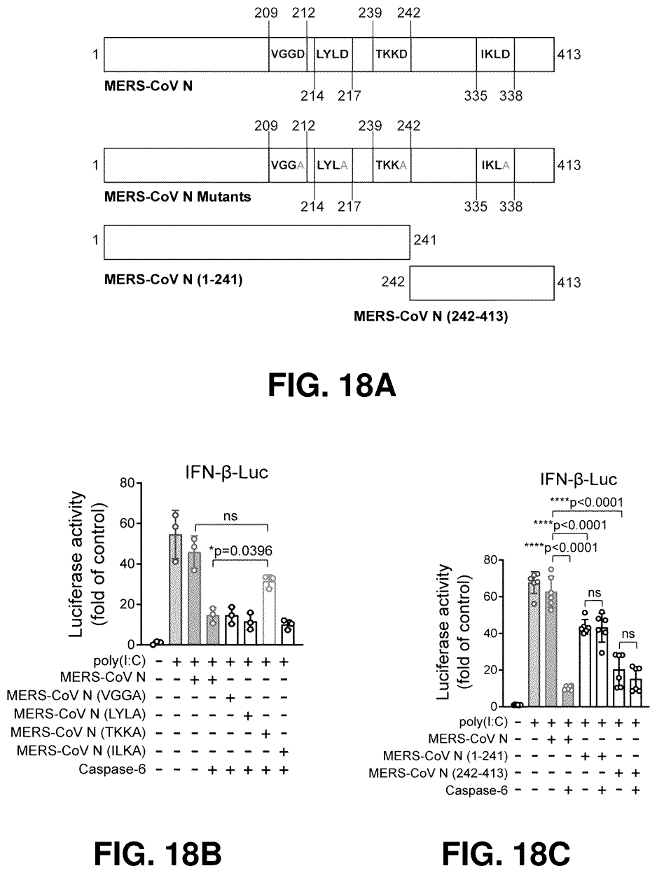

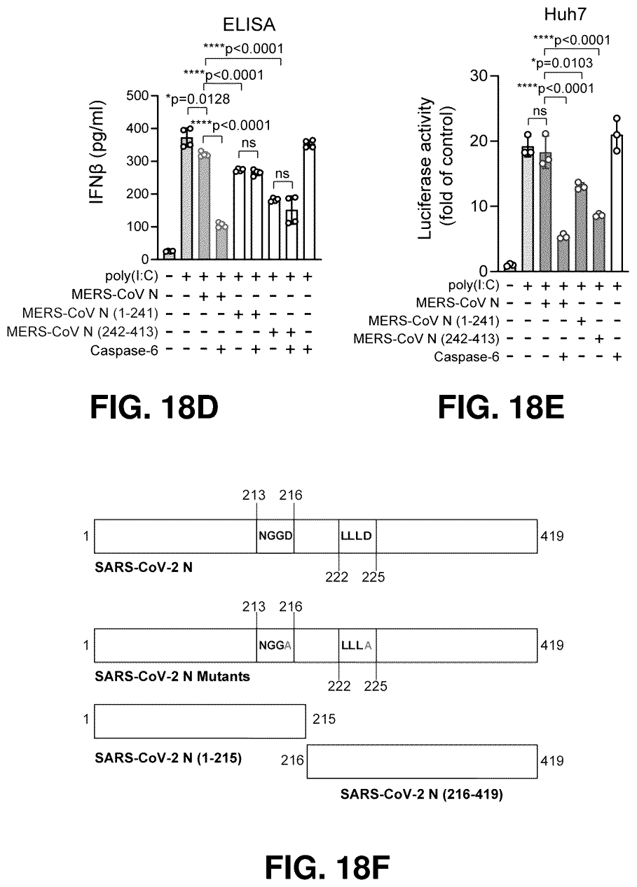

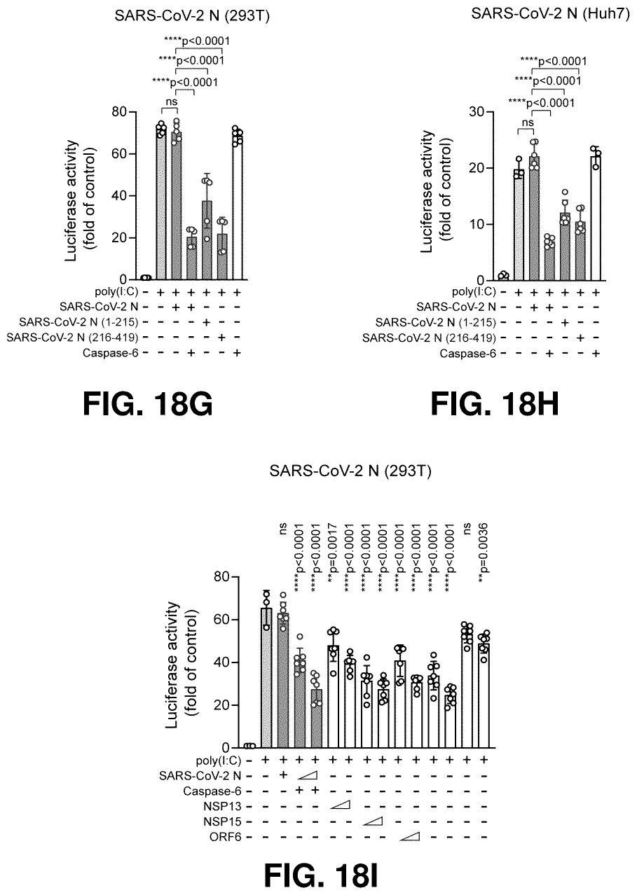

To further explain how caspase-6-mediated N cleavage regulates IFN response, the potential caspase-6 cleavage sites on MERS-CoV N were analyzed based on known caspase-6 substrate specificity (Talanian, R. V. et al. (1997) Journal of Biological Chemistry Vol. 272(15), pages 9677-9682, doi: 10.1074/jbc.272.15.9677) and the size of the cleavage fragments and generated the corresponding N mutants that potentially interfered with caspase-6 cleavage (FIG. 18A). Western blot-based cleavage assays demonstrated that caspase-6-mediated cleavage was abolished for the T239KKA242 mutant, suggesting that caspase-6 cleaves MERS-CoV N at the T239KKD242 motif. Interestingly, the T239KKD242 motif is located within the intrinsically disordered region (IDR) of MERS-CoV N that bridges the N-terminal domain and C-terminal domain of N, which are structurally conserved among coronaviruses (McBride, R., et al. (2014) Viruses Vol. 6(8), pages 2991-3018, doi:10.3390/v6082991). Similar putative caspase-6 cleavage motifs are also present in the IDR of N of other human-pathogenic coronaviruses (Table 1). In Table 1, the N-terminal domain of coronavirus N is bolded and the C-terminal domain of the coronavirus N is italicized. The putative caspase-6 cleavage motifs in the intrinsically disordered region (IDR) are in lower case letters. In IFN-β-reporter assays, caspase-6 and N-mediated IFN antagonism was attenuated when T239KKA242 was expressed in place of the wildtype N (FIG. 18B). These findings suggested that caspase-6-mediated MERS-CoV N cleavage is essential for its IFN antagonism. The N(1-241) and N(242-413) fragments were generated that mimicked the N cleavage products (FIG. 18A). Western blots analysis confirmed that the two fragments were no longer cleaved by caspase-6. Consistent with this result, in IFN-β-reporter assays and IFN-β ELISA, the two N cleavage products individually limited IFN-β-reporter activity but were no longer modulated by caspase-6 (FIGS. 18C, 18D and FIG. 18E). Similar to what was done for MERS-CoV, the caspase-6 cleavage site on SARS-CoV-2 N was identified. In keeping with the findings on MERS-CoV N, the N fragments of SARS-CoV-2 also served as potent IFN antagonists (FIGS. 18F and 18G-18I).

TABLE 1
Putative Caspase-6 Cleavage Motifs.

| Coronavirus | Amino Acid Sequence |
|---|---|
| MERS-COV N | MASPAAPRAVSFADNNDITNTNLSRGRGRNPK |
| (YP_ | PRAAPNNTVSWYTGLTQHGKVPLTFPPGQ |
| 009047211.1) | GVPLNANSTPAQNAGYWRRQDRKINTGNG |
| | IKQLAPRWYFYYTGTGPEAALPFRAVKDG |
| | IVWVHEDGATDAPSTFGTRNPNNDSAIVT |
| | QFAPGTKLPKNFHIEGT |
| | GGNSQSSSRASSLSRNSSRSSSQGSRSGNSTR |
| | GTSPGPSGIGAvggdLlyldLLNRLQALESGK |
| | VKQSQPKVItkkdAAAAKNKMRHKRTSTKSFN |
| | MVQAFGLRGPGDLQGNFGDLQLNKLGTEDPRW |
| | PQIAELAPTASAFMGMSQFKLTHQNNDDHGNP |
| | VYFLRYSGAIKLDPKNPNYNKWLELLEQNIDA |
| | YKTFPKKEKKQKAPKEESTDQMSEPPKEQRVQ |
| | GSITQRTRTRPSVQPGPMIDVNTD |
| | (SEQ ID NO: 4) |
| SARS-COV-2 N | MSDNGPQNQRNAPRITFGGPSDSTGSNQNGER |
| (YP_ | SGARSKQRRPQGLP |
| 009724397.2) | NNTASWFTALTQHGKEDLKFPRGQGVPIN |
| | TNSSPDDQIGYYRRATRRIRGGDGKMKDL |
| | SPRWYFYYLGTGPEAGLPYGANKDGIIWV |
| | ATEGALNTPKDHIGTRNPANNAAIVLQLP |
| | QGTTLPKGFYAE |
| | GSRGGSQASSRSSSRSRNSSRNSTPGSSRGTS |
| | PARMAGnggdAALALllldRLNQLESKMSGKG |
| | QQQQGQTVTKKSAAEASKKPRQKRTATKAYNV |
| | TQAFGRRGPEQTQGNFGDQELIRQGTDYKHWP |
| | QIAQFAPSASAFFGMSRIGMEVTPSGTWLTYT |
| | GAIKLDDKDPNFKDQVILLNKHIDAYKTFPPT |
| | EPKKDKKKKADETQALPQRQKKQQTVTLLPAA |
| | DLDDFSKQLQQSMSSADSTQA |
| | (SEQ ID NO: 5) |
| SARS-COV-1 N | MSDNGPQSNQRSAPRITFGGPTDSTDNNQNGG |
| (YP_ | RNGARPKQRRPQGLP |
| 009825061.1) | NNTASWFTALTQHGKEELRFPRGQGVPIN |
| | TNSGPDDQIGYYRRATRRVRGGDGKMKEL |
| | SPRWYFYYLGTGPEASLPYGANKEGIVWV |
| | ATEGALNTPKDHIGTRNPNNNAATVLQLP |
| | QGTTLPKGFYAE |
| | GSRGGSQASSRSSSRSRGNSRNSTPGSSRGNS |
| | PARMASGGGETALALllldRLNQLESKVSGKG |
| | QQQQGQTVTKKSAAEASKKPRQKRTATKQYNV |
| | TQAFGRRGPEQTQGNFGDQDLIRQGTDYKHWP |
| | QIAQFAPSASAFFGMSRIGMEVTPSGTWLTYH |
| | GAIKLDDKDPQFKDNVILLNKHIDAYKTFPPT |
| | EPKKDKKKKTDEAQPLPQRQKKQPTVTLLPAA |
| | DMDDFSRQLQNSMSGASADSTQA |
| | (SEQ ID NO: 6) |
| HCoV-NL63N | MASVNWAD |
| (YP_ | DRAARKKFPPPSFYMPLLVSSDKAPYRVI |
| 003771.1) | PRNLVPIGKGNKDEQIGYWNVQERWRMRR |
| | GQRVDLPPKVHFYYLGTGPHKDLKFRQRS |
| | DGVVWVAKEGAKTVNTSLGNRKRNQKPLE |
| | PKFSIALPPELSVVEF |
| | EDRSNNSSRASSRSSTRNnsrdSSRSTSRQQS |
| | RtrsdSNQSSSDLVAAVTLALKNlgfdNQSKS |
| | PSSSGTSTPKKPNKPLSQPRADKPSQLKKPRW |
| | KRVPTREENVIQCFGPRDFNHNMGDSDLVQNG |
| | VDAKGFPQLAELIPNQAALFFDSEVSTDEVGD |
| | NVQITYTYKMLVAKDNKNLPKFIEQISAFTKP |
| | SSIKEMQSQSSHVAQNTVLNASIPESKPLADD |
| | DSAIIEIVNEVLH |
| | (SEQ ID NO: 7) |
| HCoV-OC43N | MSFTPGKQSSSRASSGNRSGNGILKWADQSDQ |
| (YP_ | FRNVQTRGRRAQPKQTATSQQPSGGNVV |
| 009555245.1) | PYYSWFSGITQFQKGKEFEFVEGQGVPIA |
| | PGVPATEAKGYWYRHNRRSFKTADGNQRQ |
| | LLPRWYFYYLGTGPHAKDQYGTDIDGVYW |
| | VASNQADVNTPADIVDRDPSSDEAIPTRF |
| | PPGTVLPQGYYIEGS |
| | GRSAPNSRSTSRTSSRASSAGSRSRANSGNRT |
| | PTSGvtpdMADQIASLVLAKlgkdATKPQQVT |
| | KHTAKEVRQKILNKPRQKRSPNKQCTVQQCFG |
| | KRGPNQNFGGGEMLKLGTSDPQFPILAELAPT |
| | AGAFFFGSRLELAKVQNLSGNPDEP QKDVYE |
| | LRYNGAIRFDSTLSGFETIMKVLNENLNAYQQ |
| | QDGMMNMSPKPQRQRGHKNGQGENDNISVAVP |
| | KSRVQQNKSRELTA EDISLLKKMDEPYTEDT |
| | SEI |
| | (SEQ ID NO: 8) |

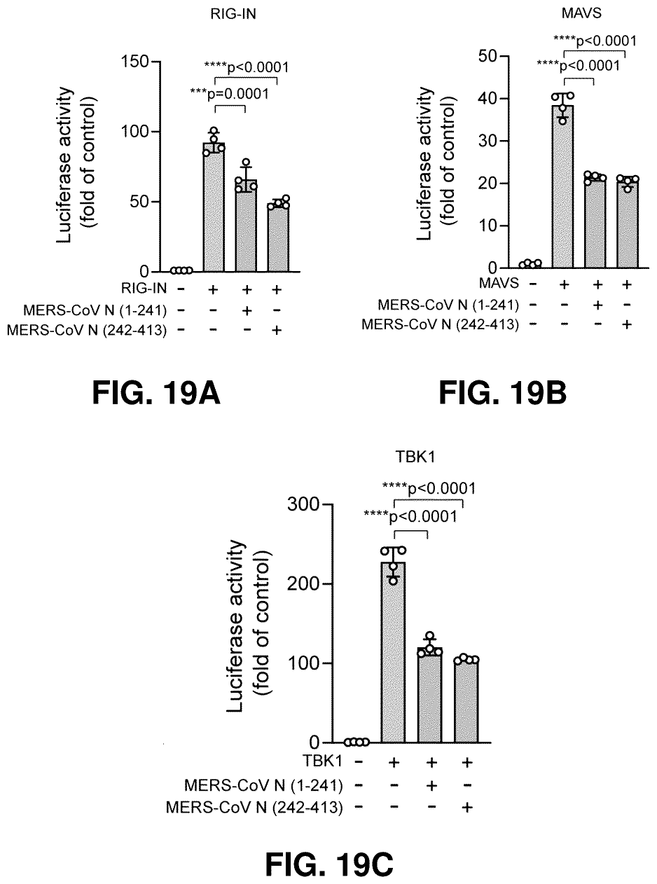

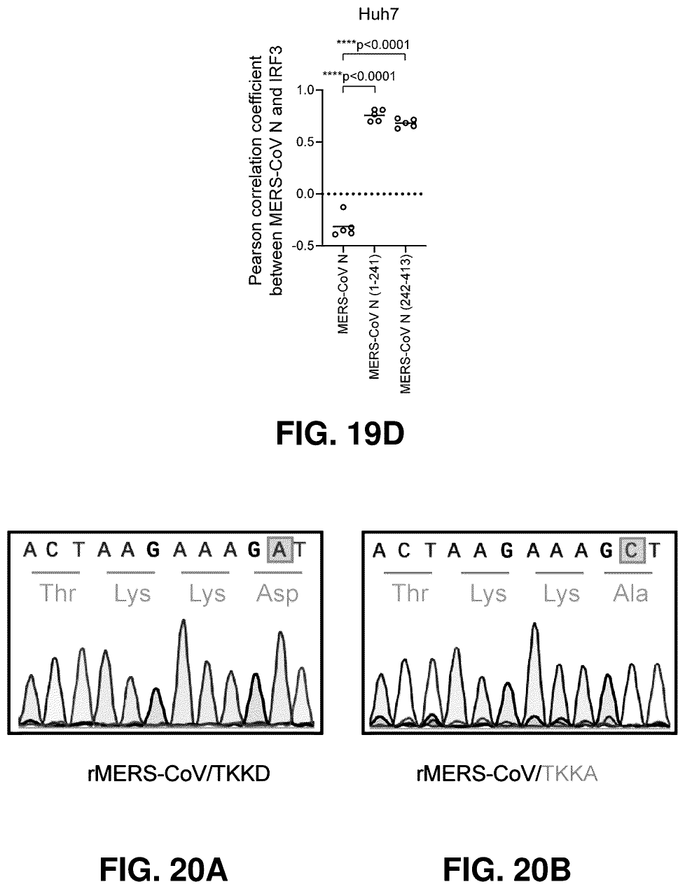

Next, to dissect how the MERS-CoV N fragments modulate IFN signaling, we evaluated the point of action of the MERS-CoV N fragments with IFN-β-luciferase reporter assays using RIG-IN (a constitutively active form of RIG-I), MAVS, or TBK1 as activators. Our results demonstrated that MERS-CoV N fragments could efficiently reduce the IFN-β-luciferase reporter activity when activated by RIG-IN, MAVS, or TBK1, suggesting that the N fragments act downstream of RIG-I, MAVS, and TBK1 (FIGS. 19A-19C). Next, the capacity N fragments of interacting with different components of the IFN signaling pathway with co-immunoprecipitation assays. This interaction between IRF3 and N, N(1-241), or N(242-413) was evaluated with co-immunoprecipitation assays using IRF3 as the bait protein. The results demonstrated that both N(1-241) and N(242-413) fragments interacted with IRF3 but not with other components of the IFN signaling pathway. Huh7 cells were transfected with expression constructs of IRF3, N, N(1-241), or N(242-413), and poly(I:C). Cells were fixed at 24 hours post transfection. Localization of N was detected with an in-house guinea pig anti-N immune serum and IRF3 was detected with a rabbit anti-HA antibody. Cell nuclei were identified with the DAPI stain. In poly(I:C)-treated Huh7 and 293T cells, IRF3 translocated to the cell nuclei regardless of MERS-CoV N expression. In stark contrast, both N(1-241) and N(242-413) fragments co-localized with IRF3 and abolished its nucleus translocation (FIG. 19D).

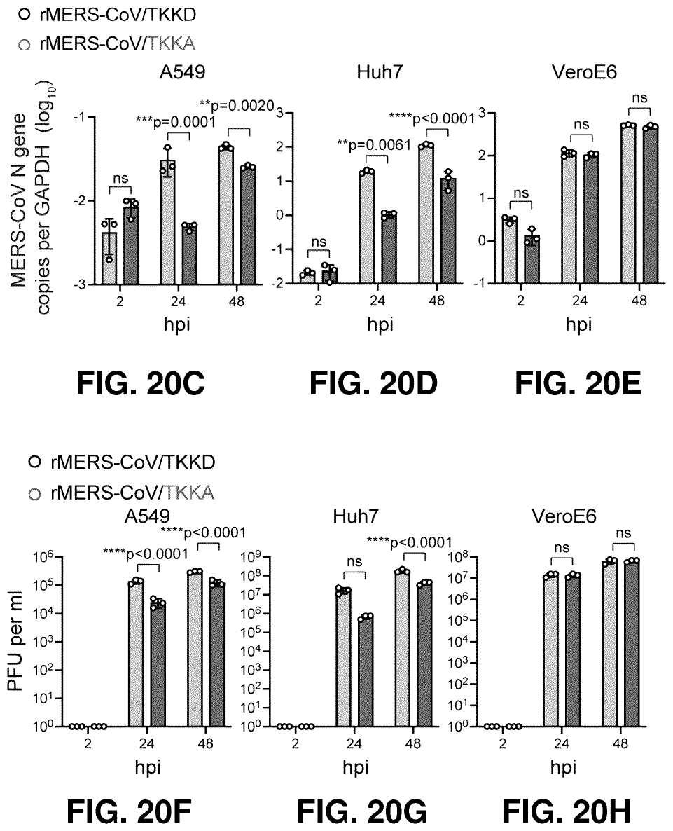

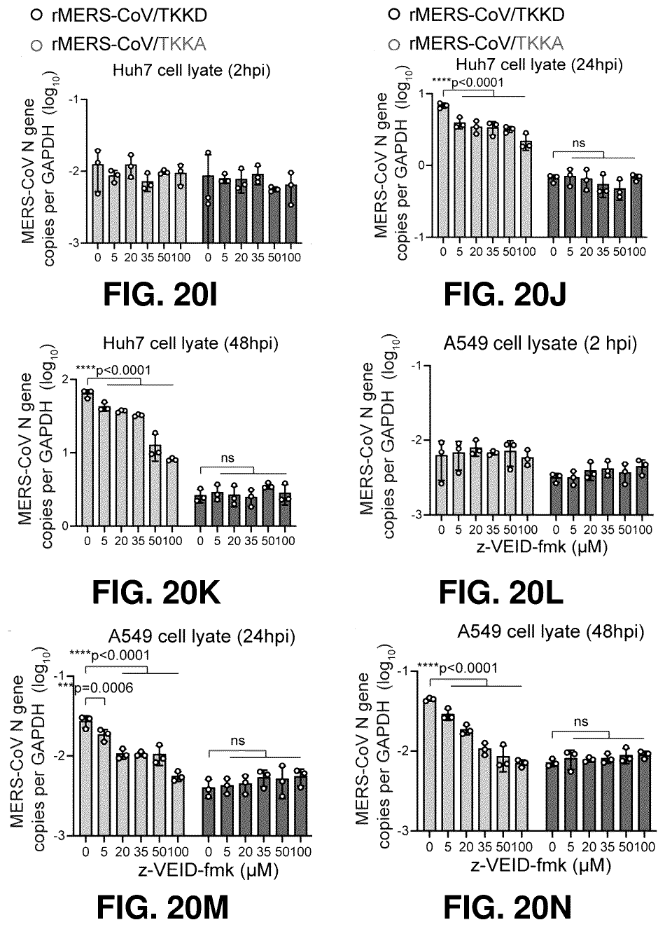

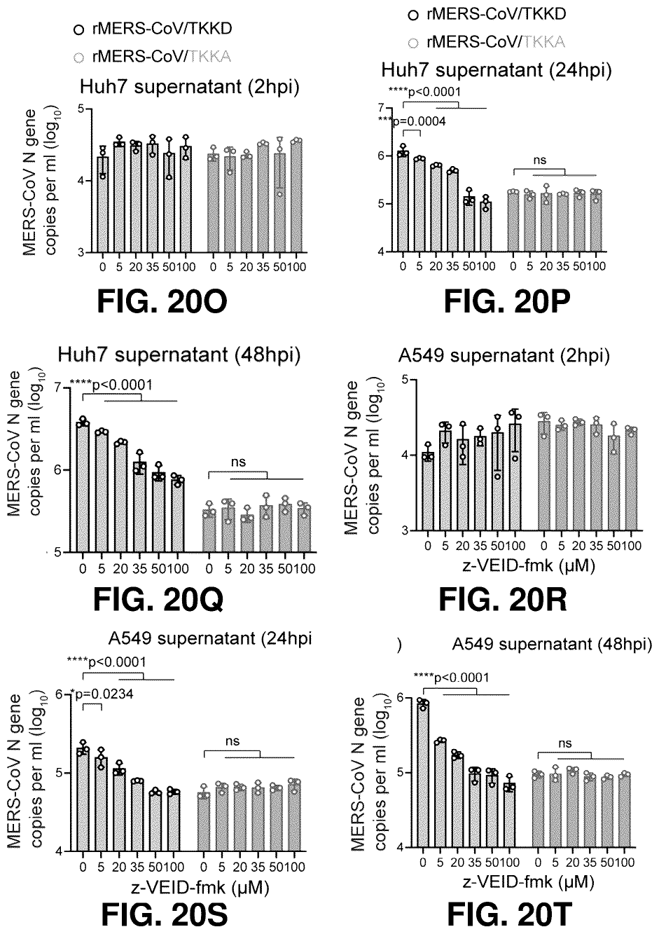

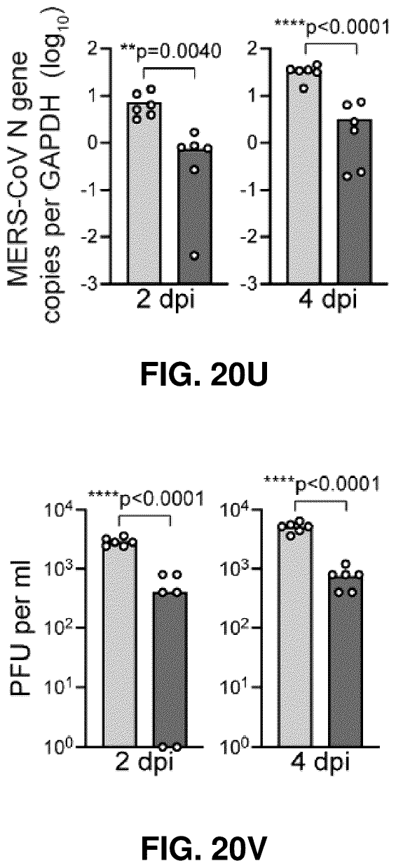

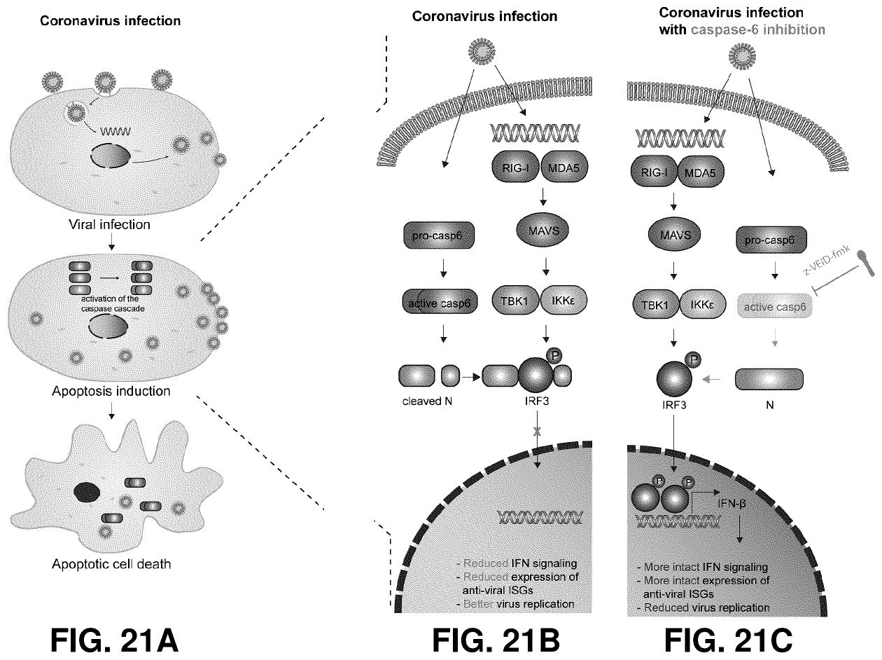

To further investigate the in vitro and in vivo importance of caspase-6-mediated N cleavage, recombinant MERS-CoV were constructed with a point mutation at the caspase-6 cleavage site on the N protein that abolished caspase-6 cleavage (rMERS-CoV/TKKA) (FIGS. 20A and 20b). A549, Huh7, and VeroE6 cells were infected with rMERS-CoV/TKKA or the wildtype recombinant MERS-CoV (rMERS-CoV/TKKD). This was accomplished by constructing a rMERS-CoV/TKKA is by introducing a ‘D’ to ‘A’ change at the TKKD motif of the MERS-CoV N gene with red recombineering. The results demonstrated that rMERS-CoV/TKKA replication was significantly reduced in comparison to that of rMERS-CoV/TKKD in A549 and Huh7 cells but not in VeroE6 cells that is defective in IFN response (FIGS. 20C-20H). Next, Huh7 and A549 cells were infected with rMERS-CoV/TKKA or rMERS-CoV/TKKD, followed by z-VEID-fmk treatment. The results demonstrated that while z-VEID-fmk remained effective against the replication of rMERS-CoV/TKKD, the replication of rMERS-CoV/TKKA was not limited by z-VEID-fmk (FIGS. 20I-20T). Finally, hDPP4 KI mice were infected with rMERS-CoV/TKKA or rMERS-CoV/TKKD to evaluate virus replication in vivo. The results demonstrated that a single nucleotide change at the caspase-6 cleavage site on N protein significantly reduced rMERS-CoV/TKKA replication and lung damage in comparison to that of rMERS-CoV/TKKD in the lungs of the infected hDPP4 KI mice (FIGS. 20U-20V). Taken together, this study identifies caspase-6-mediated N cleavage as a novel mechanism that serves to dampen the host IFN response for efficient coronavirus replication. Inhibition of caspase-6 markedly attenuates coronavirus replication and ameliorates coronavirus-induced lung pathology in vivo, suggesting that caspase-6 inhibition should be further explored as an option for the treatment of highly pathogenic coronaviruses (FIGS. 21A-21C).

Summary

In the studies of Examples 1-3, caspase-6 was revealed to be an important host factor for efficient coronavirus replication. Caspase-6 inhibition limits the replication of all evaluated human-pathogenic coronaviruses including MERS-CoV, SARS-CoV-2, SARS-CoV-1, HCoV-229E, and HCoV-OC43. In addition, caspase-6 inhibition significantly lowers the replication of highly pathogenic coronaviruses including MERS-CoV and SARS-CoV-2 in vivo, which improves the survival of MERS-CoVMA-infected hDPP4 KI mice and attenuates the body weight loss and lung pathology of SARS-CoV-2-infected golden Syrian hamsters. Mechanistically, caspase-6 mediates the cleavage of coronavirus N protein. The N cleavage products serve as IFN antagonists and interfere with activation of IFN signaling, which reduces the expression of ISGs, leading to efficient coronavirus replication. Overall, these studies reveal a novel mechanism for efficient coronavirus replication. Upon coronavirus infection, the host initiates apoptosis to eliminate infected cells and terminate virus propagation. At the same time, coronaviruses exploit a component of the activated apoptosis cascade to facilitate virus replication to maximize virus production before the cells die due to apoptosis induction. This is an elegant example of virus-host interaction that exemplifies the long-standing arms race between humans and coronaviruses.

The replication of coronaviruses including MERS-CoV and SARS-CoV-2 is known to depend on a host serine protease, transmembrane protease serine 2 (TMPRSS2), which cleavage activates the spike (S) protein of coronaviruses for efficient entry and replication (Hoffmann, M. et al. (2020) Cell Vol. 181(2), pages 271-280 e278, doi:10.1016/j.cell.2020.02.052; Chu, H. et al. (2021) Nature Communications Vol. 12(1), Article 134, doi:10.1038/s41467-020-20457). In contrast, the role of host cysteine-aspartic protease on coronavirus replication has not been explored. Previous studies have suggested that influenza viruses require a host cysteine-aspartic protease, caspase-3, for efficient translocation of viral ribonucleoprotein (RNP) complexes across the nuclear membrane, which is essential for efficient virus replication (Wurzer, W. J. et al. (2003) EMBO Journal Vol. 22(11), pages 2717-2728, doi:10.1093/emboj/cdg279; Muhlbauer, D. et al. (2015) Journal of Virology Vol. 89(11), pages 6009-6021, doi:10.1128/JVI.03531-14). In the current study, caspase-6 but not caspase-3 is shown to facilitate coronavirus replication. Caspase-6 is most known for its role as an executor caspase and its catalytic role in neurodegeneration in Huntington's and Alzheimer's disease (Graham, R. K. et. al. (2011) Trends in Neuroscience Vol. 34(12), pages 646-656, doi: 10.1016/j.tins.2011.09.001). In addition, caspase-6 has been shown to facilitate inflammasome activation, cell death, and host defense during influenza virus infection (Zheng, M. et al. (2020) Cell Vol. 181(3), pages 674-687 e613, doi: 10.1016/j.cell.2020.03.040). In the context of coronavirus research, previous studies have revealed that caspase-6 can mediate the cleavage of N protein from a number of coronaviruses, including SARS-CoV-1, transmissible gastroenteritis coronavirus (TGEV) and porcine epidemic diarrhea virus (PEDV; Diemer, C. et al. (2008) Journal of Molecular Biology Vol. 376(1), pages 23-34, doi:10.1016/j.jmb.2007.11.081; Eleouet, J. F. et al. (2000) Journal of Virology Vol. 74(9), pages 3975-3983, doi:10.1128/jvi.74.9.3975-3983.2000; Oh, C. et al. (2020) Virus Research Vol. 285, Article 198026, doi:10.1016/j.virusres.2020.198026) but the biological significance of caspase-6-mediated N cleavage is unknown. In the current study, it is demonstrated that caspase-6 mediates efficient coronavirus replication by cleaving coronavirus N proteins, which in turn serve as IFN antagonists that block IFN activation. Importantly, inhibiting caspase-6 attenuates virus replication and disease severity in highly pathogenic coronavirus-infected mice and hamsters. These findings suggest the inhibition of serine protease TMPRSS2 and the inhibition of cysteine-aspartic protease caspase-6 as therapeutic options against the infection by highly pathogenic coronaviruses including SARS-CoV-2.

IFN response is among the first line of host defense against coronaviruses infection40. Genomes of coronaviruses encode multiple viral components (Lui, P. Y. et al. (2016) Emerging Microbes and Infections Vol. 5(4), Article e39, doi:10.1038/emi.2016.33; Siu, K. L. et al. (2014) Journal of Virology 88(9), pages 4866-4876, doi: 10.1128/JVI.03649-13; Yang, Y. et al. (2013) Protein Cell 4(12), pages 951-961, doi: 10.1007/s13238-013-3096-8; Konno, Y. et al. (2020) Cell Reports Vol. 32(12), Article 108185, doi: 10.1016/j.celrep.2020.108185; Yuen, C. K. et al. (2020) Emerging Microbes and Infections Vol. 9(1), pages 1418-1428, doi:10.1080/22221751.2020. 1780953; Comar, C. E. et al. (2019) mBio Vol. 10(2), doi:10.1128/mBio.00319-19; Wong, L. R. et al. (2020) Journal of Immunology Vol. 205(6), pages 1564-1579, doi:10.4049/jimmunol.1901489; Xia, H. et al. (2020) Cell Reports Vol. 33(1), Article 108234, doi: 10.1016/j.celrep.2020.108234). including nucleocapsid protein (Chang, C. Y. et al. (2020) Journal of Virology Vol. 94(13), doi:10.1128/JVI.00099-20; Chen, K. et al. (2020) Viruses Vol. 13(1), Article 47 doi:10.3390/v13010047; Kopecky-Bromberg, S. A. et al. (2007) Journal of Virology Vol. 81(2), pages 548-557, doi:10.1128/JVI.01782-06; Oh, S. J. et al. (2021) Cells Vol. 10(3), Article 530, doi:10.3390/cells10030530) that can serve as IFN antagonists by targeting different steps of the IFN signaling pathway. Extending from these findings, the current studies further reveal host proteases as previously unappreciated factors that can modulate viral antagonism in conjunction with viral components. Taking this new knowledge into consideration, the current understanding on viral components that antagonize IFN signaling may be incomplete since viral components may be processed by host proteases, thus modulating their capacity in interacting with the host IFN signaling pathways.

Coronavirus N is an abundantly expressed multi-functional protein (McBride, R., et al. (2014) Viruses Vol. 6(8), pages 2991-3018, doi:10.3390/v6082991), which is a structural protein required for forming complexes with viral RNA and for the assembly of virus particles before release from cells. An uncleaved complex structure of N is required to carry out these functions. Once the virus enters the cell, the uncoating process will release the viral genome together with the uncleaved N protein which now no longer needs to retain the intact complex structure at this stage of the viral life cycle. At this early and critical time point, N can serve another important purpose during which the complex structure is cleaved by caspase-6 to form two fragments which can antagonize the IFN signaling pathway so that the viral replication cycle can proceed. This interesting mechanism exemplifies how coronaviruses make the best use of their most abundantly expressed protein in a time-dependent manner to ensure efficient virus replication.

Those skilled in the art will recognize, or be able to ascertain, using no more than routine experimentation, many equivalents to the specific forms of the method and compositions described herein. Such equivalents are intended to be encompassed by the following claims.

We claim:

1. A method of treating a coronavirus infection in a subject comprising administering a pharmaceutical composition comprising a caspase-6 inhibitor to the subject, wherein the amount of the caspase-6 inhibitor in the pharmaceutical composition is effective, when administered to the subject, to reduce replication of the coronavirus in the subject.

2. The method of claim 1, wherein the amount of the caspase-6 inhibitor in the pharmaceutical composition is effective, when administered to the subject, to reduce the replication of the coronavirus in the subject compared to an untreated subject with a coronavirus infection.

3. The method of claim 1, wherein the caspase-6 inhibitor comprised in the pharmaceutical composition is not a pan caspase inhibitor.

4. The method of claim 1, wherein the caspase-6 inhibitor comprised in the pharmaceutical composition does not inhibit caspase-8.

5. The method of claim 1, wherein the caspase-6 inhibitor comprised in the pharmaceutical composition is Z-VEID-FMK or AC-VEID-CHO, preferably Z-VEID-FMK.

6. The method of claim 1, wherein the caspase-6 inhibitor comprised in the pharmaceutical composition is a nucleic acid molecule selected from the group comprising a single stranded antisense nucleic acid (ssRNA), a small interfering NA (siRNA), a short hairpin RNA (shRNA), and a microRNA (miRNA).

7. The method of claim 1, wherein the amount of the caspase-6 inhibitor in the pharmaceutical composition is effective, when administered to the subject, to reduce coronavirus replication by 50% or more 24 hours following administration.

8. The method of claim 1, wherein the pharmaceutical composition is effective, when administered to the subject, to deliver the caspase-6 inhibitor at a dose from about 7.5 mg/kg/day or more.

9. The method of claim 1, wherein the pharmaceutical composition is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof following the detection of the coronavirus in the subject.

10. The method of claim 1, wherein the pharmaceutical composition is administered via oral, intranasal, intraperitoneal, intratracheal, or intrathecal administration.

11. The method of claim 1, wherein the coronavirus is an alpha coronavirus, a beta coronavirus, a gamma coronavirus, or a delta coronavirus selected from the group comprising Human Coronavirus 229E (HCoV-229E), Human Coronavirus OC43 (HCoV-OC43), Human Coronavirus NL63 (HCoV-NL63), Human Coronavirus HKU1 (HCoV-HKU1), Middle East Respiratory Syndrome Coronavirus (MERS-CoV), Severe Acute Respiratory Syndrome Coronavirus 1 (SARS-CoV-1), and SARS-CoV-2.

12. The method of claim 1, wherein the coronavirus is a non-human coronavirus.

13. The method of claim 1, wherein the coronavirus is a pathogenic coronavirus selected from the group comprising Middle East Respiratory Syndrome Coronavirus (MERS-CoV), Severe Acute Respiratory Syndrome Coronavirus 1 (SARS-CoV-1), and SARS-CoV-2.

14. The method of claim 11, wherein the SARS-CoV-2 variant is the alpha variant, beta variant, gamma variant, delta variant, epsilon variant, eta variant, iota variant, kappa variant, mu variant, omicron variant, zeta variant, 1.617.3 variant and/or lambda variant.

15. The method of claim 11, wherein the SARS-CoV-2 variant is a sub-variant of the alpha variant, beta variant, gamma variant, delta variant, epsilon variant, eta variant, iota variant, kappa variant, mu variant, omicron variant, zeta variant, 1.617.3 variant and/or lambda variant.

16. The method of claim 1, wherein the subject is a human.

17. The method of claim 1, wherein the subject has a coronavirus-induced pneumonia, coronavirus-induced bronchitis, acute respiratory distress syndrome (ARDS), acute lung injury (ALI), multisystem inflammatory syndrome in children (MIS-C), and/or multisystem inflammatory syndrome in adults (MIS-A).

18. A method of preventing or treating a coronavirus-associated disease in a subject comprising administering a pharmaceutical composition comprising a caspase-6 inhibitor to the subject, wherein the amount of the caspase-6 inhibitor in the pharmaceutical composition is effective, when administered to the subject, to reduce replication one or more symptoms of the coronavirus-associated disease in the subject.

19. A pharmaceutical composition for the treatment of a coronavirus infection in a subject comprising a caspase-6 inhibitor, wherein the amount of caspase-6 inhibitor in the composition is effective, when administered to the subject, to reduce replication of coronavirus in the subject.

20. The pharmaceutical composition of claim 19, wherein the amount of caspase-6 inhibitor in the composition is effective, when administered to the subject, to reduce replication of coronavirus in the subject compared to an untreated subject with a coronavirus infection.

21. The pharmaceutical composition of claim 19, wherein the caspase-6 inhibitor is not a pan caspase inhibitor.

22. The pharmaceutical composition of claim 19, wherein the caspase inhibitor is Z-VIED-FMK or AC-VEID-CHO, preferably Z-VEID-FMK.

23. The pharmaceutical composition of claim 19, wherein the amount of the caspase-6 inhibitor in the composition is effective, when administered to the subject, to reduce coronavirus replication by 50% or more 24 hours following administration.


SEQUENCE LISTING (65 sequences)

SEQ ID NO: 1 - DNA, 23 bp, synthetic construct
  source 1..23: mol_type = other DNA
ccctcatcttcaatcacgagagg

SEQ ID NO: 2 - DNA, 23 bp, synthetic construct
  source 1..23: mol_type = other DNA
ctcctgctcaaaattcacgaggg

SEQ ID NO: 3 - DNA, 23 bp, synthetic construct
  source 1..23: mol_type = other DNA
tggcgtcgtatgcgtaaacgtgg

SEQ ID NO: 4 - protein, 413 aa, synthetic construct
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 5 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - protein, 422 aa, synthetic construct
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 7 - protein, 377 aa, synthetic construct
MASVNWADDRAARKKFPPPSFYMPLLVSSDKAPYRVIPRNLVPIGKGNKDEQIGYWNVQERWRMRRGQRVDLPPKVHFYYLGTGPHKDLKFRQRSDGVVWVAKEGAKTVNTSLGNRKRNQKPLEPKFSIALPPELSVVEFEDRSNNSSRASSRSSTRNNSRDSSRSTSRQQSRTRSDSNQSSSDLVAAVTLALKNLGFDNQSKSPSSSGTSTPKKPNKPLSQPRADKPSQLKKPRWKRVPTREENVIQCFGPRDFNHNMGDSDLVQNGVDAKGFPQLAELIPNQAALFFDSEVSTDEVGDNVQITYTYKMLVAKDNKNLPKFIEQISAFTKPSSIKEMQSQSSHVAQNTVLNASIPESKPLADDDSAIIEIVNEVLH

SEQ ID NO: 8 - protein, 448 aa, synthetic construct
MSFTPGKQSSSRASSGNRSGNGILKWADQSDQFRNVQTRGRRAQPKQTATSQQPSGGNVVPYYSWFSGITQFQKGKEFEFVEGQGVPIAPGVPATEAKGYWYRHNRRSFKTADGNQRQLLPRWYFYYLGTGPHAKDQYGTDIDGVYWVASNQADVNTPADIVDRDPSSDEAIPTRFPPGTVLPQGYYIEGSGRSAPNSRSTSRTSSRASSAGSRSRANSGNRTPTSGVTPDMADQIASLVLAKLGKDATKPQQVTKHTAKEVRQKILNKPRQKRSPNKQCTVQQCFGKRGPNQNFGGGEMLKLGTSDPQFPILAELAPTAGAFFFGSRLELAKVQNLSGNPDEPQKDVYELRYNGAIRFDSTLSGFETIMKVLNENLNAYQQQDGMMNMSPKPQRQRGHKNGQGENDNISVAVPKSRVQQNKSRELTAEDISLLKKMDEPYTEDTSEI

SEQ ID NO: 9 - DNA, 19 bp, synthetic construct
  source 1..19: mol_type = other DNA
gcccctgaatgcggctaat

SEQ ID NO: 10 - DNA, 20 bp, synthetic construct
  source 1..20: mol_type = other DNA
attgtcaccataagcagyca

SEQ ID NO: 11 - DNA, 25 bp, synthetic construct
  source 1..25: mol_type = other DNA
cggacacccaaagtagtcggttccg

SEQ ID NO: 12 - DNA, 22 bp, synthetic construct
  source 1..22: mol_type = other DNA
aggaaaggtgcttccgaggtag

SEQ ID NO: 13 - DNA, 21 bp, synthetic construct
  source 1..21: mol_type = other DNA
cttctaaccgaggtcgaaacg

SEQ ID NO: 14 - DNA, 22 bp, synthetic construct
  source 1..22: mol_type = other DNA
ggcattttggacaaakcgtcta

SEQ ID NO: 15 - DNA, 20 bp, synthetic construct
  source 1..20: mol_type = other DNA
cgtactcctaagccttctcg

SEQ ID NO: 16 - DNA, 21 bp, synthetic construct
  source 1..21: mol_type = other DNA
tcgactagggttaagaagagg

SEQ ID NO: 17 - DNA, 23 bp, synthetic construct
  source 1..23: mol_type = other DNA
cgatgaggctattccgactaggt

SEQ ID NO: 18 - DNA, 27 bp, synthetic construct
  source 1..27: mol_type = other DNA
ccttcctgagccttcaatatagtaacc

SEQ ID NO: 19 - DNA, 25 bp, synthetic construct
  source 1..25: mol_type = other DNA
caaaaccttccctaagaaggaaaag

SEQ ID NO: 20 - DNA, 22 bp, synthetic construct
  source 1..22: mol_type = other DNA
gctcctttggaggttcagacat

SEQ ID NO: 21 - DNA, 21 bp, synthetic construct
  source 1..21: mol_type = other DNA
accagaatggaggacgcaatg

SEQ ID NO: 22 - DNA, 25 bp, synthetic construct
  source 1..25: mol_type = other DNA
gctgtgaaccaagacgcagtattat

SEQ ID NO: 23 - DNA, 20 bp, synthetic construct
  source 1..20: mol_type = other DNA
cgcatacagtcttrcaggct

SEQ ID NO: 24 - DNA, 24 bp, synthetic construct
  source 1..24: mol_type = other DNA
gtgtgatgttgawatgacatggtc

SEQ ID NO: 25 - DNA, 28 bp, synthetic construct
  source 1..28: mol_type = other DNA
ttaagatgtggtgcttgcatacgtagac

SEQ ID NO: 26 - DNA, 22 bp, synthetic construct
  source 1..22: mol_type = other DNA
ggactgaggaagacaaccaggt

SEQ ID NO: 27 - DNA, 20 bp, synthetic construct
  source 1..20: mol_type = other DNA
attccacccatggcaaattc

SEQ ID NO: 28 - DNA, 20 bp, synthetic construct
  source 1..20: mol_type = other DNA
cgctcctggaagatggtgat

SEQ ID NO: 29 - DNA, 22 bp, synthetic construct
  source 1..22: mol_type = other DNA
agaagcaggcaatcacagaaaa

SEQ ID NO: 30 - DNA, 23 bp, synthetic construct
  source 1..23: mol_type = other DNA
ctgaaaccgaccatagtggaaat

SEQ ID NO: 31 - RNA, 19 nt, synthetic construct
  source 1..19: mol_type = other RNA
cacatgggccgactctcag

SEQ ID NO: 32 - DNA, 21 bp, synthetic construct
  source 1..21: mol_type = other DNA
ccacactttaaccgtgtccac

SEQ ID NO: 33 - DNA, 20 bp, synthetic construct
  source 1..20: mol_type = other DNA
gaacatgctgaccaagcaga

SEQ ID NO: 34 - DNA, 20 bp, synthetic construct
  source 1..20: mol_type = other DNA
cagttgtgtccacccttcct

SEQ ID NO: 35 - DNA, 20 bp, synthetic construct
  source 1..20: mol_type = other DNA
atgtcgtctggtccctgttc

SEQ ID NO: 36 - DNA, 20 bp, synthetic construct
  source 1..20: mol_type = other DNA
gtcatgaggatgcccagaat

SEQ ID NO: 37 - DNA, 18 bp, synthetic construct
  source 1..18: mol_type = other DNA
cgccgcattgaccatcta

SEQ ID NO: 38 - DNA, 20 bp, synthetic construct
  source 1..20: mol_type = other DNA
gacattagccaggaggttct

SEQ ID NO: 39 - DNA, 24 bp, synthetic construct
  source 1..24: mol_type = other DNA
actgtctcaggaacaccaaggtca

SEQ ID NO: 40 - DNA, 26 bp, synthetic construct
  source 1..26: mol_type = other DNA
ccaggttatccagcacattcacctca

SEQ ID NO: 41 - DNA, 20 bp, synthetic construct
  source 1..20: mol_type = other DNA
cggttccatctcgccattca

SEQ ID NO: 42 - DNA, 20 bp, synthetic construct
  source 1..20: mol_type = other DNA
gcggctatgactgaggaagg

SEQ ID NO: 43 - DNA, 20 bp, synthetic construct
  source 1..20: mol_type = other DNA
gagtggaagtgtggacaggg

SEQ ID NO: 44 - DNA, 20 bp, synthetic construct
  source 1..20: mol_type = other DNA
tcaagacggaacttctgccc

SEQ ID NO: 45 - DNA, 28 bp, synthetic construct
  source 1..28: mol_type = other DNA
cgacttcaacagcaactcccactcttcc

SEQ ID NO: 46 - DNA, 27 bp, synthetic construct
  source 1..27: mol_type = other DNA
tgggtggtccagggtttcttactcctt

SEQ ID NO: 47 - DNA, 25 bp, synthetic construct
  source 1..25: mol_type = other DNA
tggagtcacagaaggagtggctaag

SEQ ID NO: 48 - DNA, 25 bp, synthetic construct
  source 1..25: mol_type = other DNA
tctgaccacagtgaggaatgtccac

SEQ ID NO: 49 - DNA, 20 bp, synthetic construct
  source 1..20: mol_type = other DNA
atgacgggccagtgagaatg

SEQ ID NO: 50 - DNA, 20 bp, synthetic construct
  source 1..20: mol_type = other DNA
gaggctctctgctgtccatc

SEQ ID NO: 51 - DNA, 21 bp, synthetic construct
  source 1..21: mol_type = other DNA
ggctcagccagatgcagttaa

SEQ ID NO: 52 - DNA, 24 bp, synthetic construct
  source 1..24: mol_type = other DNA
cctactcattgggatcatcttgct

SEQ ID NO: 53 - protein, 293 aa, Homo sapiens
MSSASGLRRGHPAGGEENMTETDAFYKREMFDPAEKYKMDHRRRGIALIFNHERFFWHLTLPERRGTCADRDNLTRRFSDLGFEVKCFNDLKAEELLLKIHEVSTVSHADADCFVCVFLSHGEGNHIYAYDAKIEIQTLTGLFKGDKCHSLVGKPKIFIIQACRGNQHDVPVIPLDVVDNQTEKLDTNITEVDAASVYTLPAGADFLMCYSVAEGYYSHRETVNGSWYIQDLCEMLGKYGSSLEFTELLTLVNRKVSQRRVDFCKDPSAIGKKQVPCFASMLTKKLHFFPKSN

SEQ ID NO: 54 - protein, 276 aa, Mus musculus
MTETDGFYKSREVFDPAEQYKMDHKRRGVALIFNHERFFWHLTLPERRGTNADRDNLTRRFSDLGFEVKCFNDLRAEELLLKIHEVSTSSHIDADCFICVFLSHGEGNHVYAYDAKIEIQTLTGLFKGDKCQSLVGKPKIFIIQACRGSQHDVPVVPLDMVDHQTDKLDNVTQVDAASVYTLPAGADFLMCYSVAEGYYSHRETVNGSWYIQDLCEMLARYGSSLEFTELLTLVNRKVSQRRVDFCKDPDAIGKKQVPCFASMLTKKLHFCPKPSK

SEQ ID NO: 55 - protein, 275 aa, Mesocricetus auratus
MTETDGFYKSREVFDPAEQYKMDHKRRGIALIFNHERFFWHLTLPERRGTSADRDNLIRRLSDLGFEVKCFNDLKAQELLLKIHEVSTSSHVDADCFLCVELSHGEGNHIYAYDAKIEIQTLTGLFKGDKCQSLVGKPKIFIIQACRNQHDVPVVPLDVVDHHTGLPDNVTQVDAASVYTLPAGADFLMCYSVAEGYYSHRETVNGSWYIQDLCEMLARYGSSLEFTELLTLVNRKVSQRRVDFCKDPGAIGKKQVPCFASMLTKKLHFCPKSSK

SEQ ID NO: 56 - protein, 20 aa, synthetic construct
AAVALLPAVLLALLAPVEID

SEQ ID NO: 57 - DNA, 52380 bp, Homo sapiens
  source 1..52380: mol_type = genomic DNA
tgagaccctgtctcaaaaaaaaaaaaaaaaaaaaattgggttaaacagttgaatgcaaacaaaaaataagatgcatgagactaaaactggaaatctgtagatatatatgtgtgtatgtaaatatgctacaggttgtgagagtttggtgatttcatctagcatacatttcagtgctcacgagggatatgcttgttgctgtgctgtaatgtgtaacataggtgaaaagagcaatgcagttgcccaacagtaatcccattttcttgttttgcagcatgtgaaacaacagtggagctcttgtttgacagtgttatacatttaggttgtaaaccatatctggacagccaacgagccgcatgcaggtgtcattatgaagaaaaaactgatctttaaaggagatgccgacagctagtgacagatgaagatggaagaacatacctttgacaaataactaatgtttttacaacataaaactgtcttatttttgtgaaaggattattttgagaccttaaaataatttatatcttgatgttaaaacctcaaagcaaaaaaagtgagggagatagtgaggggagggcacgcttgtcttctcaggtatcttccccagcattgctcccttacttagtatgccaaatgtcttgaccaatatcaaaaacaagtgcttgtttagcggagaattttgaaaagaggaatatataactcaattttcacaaccacatttaccaaaaaaagagatcaaatataaaattcatcataatgtctgttcaacattatcttatttggaaaatggggaaattatcacttacaagtatttgtttactatgaaattttaaatacacatttatgcctagaaggaacggactttttttttctattttaattacacataatatgtaattaaagtacaacataatatgttgtttctctgtagcccgttgagcatatgagtaagtcacatttctattaggactacttacaaggacaaggtttccatttttccagttgtaaaattggaaccatcagctgataacctcgtagggagcaaccccaggatagctaagtgttatgtaatatgcctagaaggtgatgtgaatgcgattcagaagcatagccactcccattttatgagctactcacatgacaaatgtcatcttttgctataacctttgccaagttagagaaaagatggatttaatgagataaatgaaaagatatttaacctaatatatcaaggcactatttgctgttatgctttgttatttatttcccagcacttgttccttattgtagattttttaaagactgtaaccttttactaactgtggtcttactaaaatttgtgcttgatactgcttttcaaaaagcctttaattagagccaaaaggatggaaaaggcaagatataaatgccttttatagatctcttatttacattgaaaattattaccatatgtttagagcaaatccaagaaaacttcaacagcttctgaagatgtctatgaatgttgaaaacttttcaatctcttggaatgctcagttatgttcctagaccggtctttgctgactactggttgttaacctttccctagcctgggacctcaagccatatatatcctttgggtgacccatggccaaagttattaagatgaactgactttcaaagtcagagaaggacagcatagggagaggcggttatttgtaagtcattacaggtagaacagggcagaaggaaaagtatgttctggagaaagggccatgttcctaactttggagatatgtcattgccgggaacctagtatcttccaacttgaattggtggcagctgttccagtgagacaaggcacatgtatgccttgtggctaagtgagcaaactgggtttccacttaaatgtttgggaccctcaattgattctttatttcaaacctttataaaaggtacagttttgtaagccattattaataattaatgcttatcggctgggcacagtggctcacacctataatcccagcactttgggaggctgaggcggttggatcacttgaggtcaggagtttgagaccagctggccaacatggtgaaacagcgtctctactaaaaatacaaaaatttgccgggcgtggtggcgcatgcttatagtctcagctactcaggaagctgaggtacgagaatcacttgaacccaggaggtggaggttgcagtgagctgagattgtgccactgcactgcagcctgggcaacagagcaagactctaaaaaaaaaatggtttatcaatatttactattgaaatatttgtccaggtaactttacacttacaatgaattcatggattttgttagcagcattggctttctcaaaaggacaaactcaatatcgttataaaatataattcgtgatcacaaattatacaaaaatcagtagaaacagttttttatgttcagattaaaaaaaaaaacttgggataattttagatttacagaaaagttgcaaagatacatggagagcttctgtgaccactcacccagttcccccagtgttaaccttttatttaaccatgaagcatttgtcagaagctaagtaaccagcaatggcaattactattaacggaactttgactttatttttcagattgtactagttttttaattaatgtcatttttcttttccaggatccaatctaggataccacactgaattagtcgtcatgcctaattagcctctggtctgtgatagttccacagtctttctttttcataaccttgacagttttgaggagtactggtcaggtgttttgtagaatattcctcaatttgggtttgtctgatgttttcctcatggttagagtggggttatagatttttaggaagaataccagaggtgaaggtccttctcactgcatcatgtcaggagttacatgctatcagcttgatggtgtattaactttggacacttggttaaggtagtgtgtgttggttttttgctgctgaaaattactgttattttccctttccatacttctgttctttggaaaacagtcactaagtccagtcatgggaggtggtgggtgggaaagattacattcaaccccctggaagtgggaatatccatatgtagtatttggaatttttctatatggaaaatttgtttctccctcccaccctaatttgtttacatcagtatggactcatgtatattttgtattttgggtaacacagtatttattttgttgcttaagttgtccagcttggctattaggagttctgccaggttggctactatgtccctttgatgtgcccatccttttgatttttgagcacttcttactttctggcactacaagatgctccaggttcatcttggatattccctgccccaaccctagaatccctagaatcaacccctgctccaaagagccctggttccttttgttggagaatcatacttagaaaccaagatctgggcattagatgtgcttgttgctactgggatgtcactgtttgtagcagagttgagaaatatgtatgtatattaatccatgcatatgtacacatctataattatttatgtgtgtacaaagctaaacatgagtttgtactgccgtcttcaactcaaaatttgtcccaaaattttgtggcatatgtttagattttaaagttgatattttccctattgacagaataaactcattaaaagagcaaaaaaaaaaaaaaaaaaaaaaaggcaagaactaactgtcagcagctctttgcaagaaatgtaatattgatactttaaaataatactgccttcaaatgatgcctgtctctttaaaatcttttgacatagttcttccagggacagatatggctgtatttatttcatagatggagaaactgagcacagagctgtaatgaagacagaattgagatataagggcaaaagctaattaaacgcatcctcacaggtagcctttctttcagtgaacctgtagactagtccagtaatacttattaaaattagttgttagaggctgggcatggtggttcaagcctgtaatctcagcactgtgggaggccaaggcggacagatcactcaaagtcagaagttcgagaccagcttggccaacatggcaaaaccctgtctctactaaaaatacaaaaattagttgggtgtggtggcacatgcctgtaatcccagccactcgggaggtgaaggcacaagaattggttgaacctgggaagcagaggttgcagtgagctgagattgcactgctgcactccagcataggcgtcagaataagactctgtcttggaaagaagaaaaaaaattgttagcctaaaaccaactctctctaaatttaaaagagtgattatttatcttagtatttatagaagtatgagattatcagaaggtgcgatgcacaatgctcattattttatcctagtacaagacacgtataacatctaacagtaaagaagatggaatctttctataaaaacaattctgttattaatagttgactgacttcagtaactttgaggagtagaatgaaacttattaggcaagatctttccacatacactacattgtattgttcaaaattcatagaaattacttgcaatatattgatttaagccttatgtagtgactgttgctatatttgaagtagcatttttagtttgctgtttttcctcactgcttaatcctacaaaaaccagccatctgcctaaaaaataaaagagcaaaaatgaatcagtgcaaccggtatacgaaaaataataattattaataaaactgtaaagggatcactagtgtcatcttttatatatgtcacaatgatgttagactttttgcctcaggcttacttattcagtttccataacatccttcaaaaatctccttgttgacaacttgacaaacatttctctgttggctttcaagttttaagtatagcaatcagaatcatcatagaaattaatatttctatgaattctgtaagaaaaaatattaaacaccaaaatcagtgaccaaaatttttaagttgtagaattatctctgaacttaactttattccctatatttcattcctatgataggtgacaagccaagaccttcagttttaataagttccacttttatttatattttaaaataatatattgcctttgaataatgcctatctccttaaaatcttttggcacatttcttccagggacagagatggctgtttatttcataaaggagacattgagcacagagaagctgtaatgaagacacgattgagatatgagggcaaaccagtttcaataagtcacttttatggaaaactattcagatataggaagaatcaagtcatcctgtcagttaacttagtgtatctgaattttaatggatttgctgtgggaatatttttctttgagagggaagagagtgcttggtaggattccagtctatgttggtggttctcaaacatgagcatgcgtgagactcactggcaggcttgttaaaacacactgctggaccccacccccagagtttctgattcagtaggtctggggtagacaggagaatttgcatttctaacaagttctcagatgatgatactgctggttcatagaccacagcttgggaagtagtttaggtaatcttcagtgctgaaaataactaatgtactgaaagcttgctgtgggttcagtactttcataggagtttgcaatttactgtctcaaaaactctggaatagatttggacctcattttacaacaaagagtttgaagctatgagaggttaagttgcattacctgtcacatatctaacagcaaataaaatggggaagccgtgagtagaacctgggcagcctgtttcccagtcccaggcactcagccattttattgtggctagctgttgctttaagaggctaaaattatacatttgtaattctgaaataagtacccaagaaaatgttctgtatttatgtttttagacaaaatcctgatcagtggtgaataaggggaaaaaaaggtagacaaaatattgttttaaaatacttaaccacatagactatagtagtaatgttgagataataagatacttatggagaaaaaatgacaaattccttccatctgctcttttcagataagtagaacgtcagccaactggagttccactcttgtcgagggaatgttttcaggactcagagaccctgaaagtgaggccaaaggactggaagttctggtggtcagataactcattcagactggataaaagccagagtgtaaatgtgaaaaccctgaattgtagtcaatggctaaatattgagccacatgcaaggcaactggggtaggtaatgaggaaaccaaaaaatgccactgctgtttccaacgagtacaacaagatggtaataaccaatattcattgagctcttcacatggcaggcagtattctgagtccttcacagtcacttaacccaatcatcacaaccaccatacaagggaaatatggaaattccatgtctttgttgcaggataagttactaagacagtgatttctgaaatattttatgcatacaatgaacagtaaatggtttgagtaaaaacccttctttgatttcgtactctaatgacatcaagacttgaaccacaggcacatgtagaagttaccaaattagattttttctctcccaaataatagtatgagatgagttccttatacttatttcttaagtattcattatacttattttaatatattagaagtggtgccttaacacattttaatttttttcccactctaaggttgcttacttttaaaattacaaggtccagaggaattacaaaagataaagccaccgtttcatcaaatctgaaagcattgtcaaagtaaattttttacaatagccaaccacccaaggaggagctaaattaaatccaacattgagcaccccatggcaccttgttggcacactgtttggctgctatttttggggtaggacaacatgctctccccaggaaacaccattatctagagaccctcatcattctgtgaatttctgcatggttctgtccctgaattagctctacctatgaaagccagatttgaaaaatccaggagaagccaggtgtcgtggctcatgcccgtaatcccaacattctgggaggccaaggtgggcagataccaggtcgggagtttgataccagcctgaccaacatggagaaaccccatctctactaaaaatacaaaattagccaggcatggtagtgcacgcctgtaatcctagctacttgggaggccaaggcaggagaatcgcttgaacccgggcggcagaggttgcggtgagctgagatcatgccattgcactccagcctgggcaacaacagctaaactgtctcaaaaaaaaaaaaaaaaaatccaggagttacctccagtttactttggcccaagtttcatatacaaagaacatttaaaataagttactgcaagtcctacaaagtaacttgcactggggaaaggactacacatgaaacaggaacatatatgccaccccctatgggatacctcagagggatttcgagtttggttccagaccaccaataaaagagtcacacaaattttctggtttcccagtgcatatgaaagtttttcatactaaagtgtagtctgttaagtgtgcaataacatgtctaaaaaaataatgtatataccttaattttaaaatattgctaaaagatgctagggatcatctgagccttcagcgagttataaatgttggtgtggggtattgttgagggctgctgactgattagggtggtagttgctgaaggctgggatgactggcaatctcttaaaataaggcaataaggaagtttgcttcatttttttgtttgttttgagatggagtctcgttctgtcactcaggctggagtgcagtggcatgatcttggctcactgcaacctctgcctcctggttcaagtgattcttgtgcctcagcctcctgagtagctgggattacaggtgcctgccaccgtgcccggctaatttttgttatttttagtagagacagggttttgccatgttggccagtctgggctcgaactcctggcctcccaaagtcctgggattacaggcatgagccactgcacctggccatccattgattgttccattcactaaagatttccctgtagcatgtgatgctgtttggtagcattttatccacagaccttctttcaaaattggggtcaatccccccaaaccctgccactgctttgtcaactaataccctgaatcctttgttgtcatttaaacacaattcacagcatattcaccaggagtagattctatctcaataaaccactttctttgctcatccgtaaaaagcatctcctcatctatcaaagttttatcatgagatggcaacaatgcagccacattttcaggccccacttccaattctatttgtcttgctacttctgccacatctacagtgacttccctcactgaagtcttgaacccctcaacatcagtgatgaggattggaattaacttctttcaaactcctgttaatgtggagatatatgcatatacctatgtataatgtatgtgtgtgtgtatatgtgtgtgtgtatatatatatatatatatatatatatatatatatatataaaatggataggtatgttgtcctgcctgtaaaataccacccagtgtgccaattgaaatatatataatttttaaatatttatatatgtattttatatatattatatatatatatatatatatatatatatattttttttttttttttttttttttttttaaagagtgtcttactctgtcacccagactggagtgcagtggcacagtcagctgactgcagcctcaaacttgggctcaaataatcctgccatcttagtgtcctgagttgctgggaatacaggcatgtgccaccactcttggctaattaaaaaaaaaaaaaaattgtagagacagggtcttgcattgttgcctaggctggtctcaaaactcctggcctcaagcaatccaccttggcctcccaaagtgctgggtttacaggcatgagccactgtggctgatacatgtacttttaatttttcgtttttctttttttccccctctagtctctagttacataccgtaagtggatattttgacctcccatgaatcacaaatgttcttaatggcatctcgaatgatgaatcctttccagaagattttcaatttactttgctcagaactatcagaggaatcatgatgacagctatagccttatgaaatgtattttgtaaataagacttgaaagtcaaaatttctccttgatccctgggcttcagaatggatgttgtgttagcaagcacgataacagcatcagtctctgtacatctctatcagaactcttggatgaccaggtgtattgtcaatgaggagtaatattttgaaaggaacctttcttctgagcactagagctcaacagtgggctttaaatattctctaagccatgctgtaaatagatgtgctgtcactaactagcctttgttggtccatttatagatcatatttagcataattcttaagggcactaggattttcagaatggtaaaggaatgttggctttaaagtcatcagttgtattagcccctaacaagagcatcagcctgttctttgaagctttgaagacaggcattgacttctctctagctatgaaagtcttagatggaatcttcttccaatagaaggccgtttcatatacatttaagatctgttgttttggctgggcacggtggctcacacctgtaatcccagcactttgggagaccgaggcgggcagattgctcaaggtcaggagttcaagaccagcctggccaaacatggtgaaactctgtctctactaaaaatacaaaattaacctggacatggtggcacatcctgtaatcccagttacttgggaggctgaggcagaagaatcgtttgaacccaggaggcagaggttgcaaaagatcgcaccactgtactccagccttggtgacaaagcaagactgtctcaaaaacaaaagattgttgtttagtgtagctgcctttatcaatgattttagctagatctggataacctgatgcagcttctacgttagcacctgctgcttcaccttgcacttttatgtcatgaagatagtttctttccttaaacctcaggaaccatcctctctgctagcttccagcttttcttttgcaattccctcacctctctcagcctccatagaattgaagagttagggccttgctctggattaggctttggcttaagggaatgttgtggctgtttttatcttctatccagaccaactagaactttctccatatcagcaataaggctgtttagctttattgtcttttgtgtgtttactggagtagcatttttaatttccttcaaaattttttcctttgcattcatgacttggcttttctcctttgcactcacaacttggctaactggagcaagaagcctagctttctgcctaccaacattacttcctcactaacttaatcatgtttgcttttgatttaaagtgacaggctcttcctttcatttggacacttagaggcctttgtaggattattagttggcctaatttcaaaattgtgtttccaggaatagggaggccccaggagggggagagagaagggaaagttcggtggaacagtccaaacacatacagggcttatggattaatttccccttcctttatgagcgcggttcatggtgccccaaaacaattgcaataatattaaagattactgatcacagatcacaacagcagacataataacaatgaaaaattttgaaatattgcgagagtaccaaaatgtgacacagagacacaaagcgagccaatgttgaaatacaggcagtatctgctaagcggtataaaacaatctatgcctgtacctctagaaagtacaggcatagcaaagtaaggaatagcaaatgtttggggcagctgggattaaatgagtccgagctcacacaagtgatgtgaaattgcctcgggctaggcggtctgtgtgtccccccgggtggcaactggcatgtccccagggcacggttagtcccagcgggcctggaggcgaagaccgcgtacctctccctgcccccgcgctcttggtccccgcgggctccggagcgactgggaggagctttaactctgggaaggaccagcccgatccacgggagcccgggcggcgcgcaatcagaaccaactggccccacgctcgggggcagggggcggggccgccgaggaagggccgagggcggggccgggcccgggagcctgtggcttcaggaagaggagggcaaggtgtctggctgcgcgtttggctgcaatgagctcggcctcggggctccgcagggggcacccggcaggtaggcagggggcgtcgactgggcaccgagcaggtctgcgtcttgctccggctggaggggaaacgagggttagtggaccgggcgcgcggaccggagtggggccgcgggttccttcggctttgcagggcggcctggagaagttgcactcttggattgaagcgctcagggcgggtggggacggtttcgcgcttcagtctaagaaacactcggggacctggctgccgcctcctggcggtcccgagtagcgacgccggcgccggggcgtgcccctgccgcctttgtcctccggggcgatgagcgctttcggggcgccccggctgtttaaaccttaaaaggctgtccccacgcgggcctggcgttcccccacacctggccgtttgaaatgttgggggttggatcttttgtttgctcacttcgaaaagtgaaaaggaggggttgttccgccgagtcataatgaaaacataacaggcccgcggggtgcccgggccaagaccctgggtcctcccgggcttcagctggaggcggccagttcctggcggccagcacaccccggtatctctcttcttcatccaggaaacctgctgctgtctgtcggtttgagccaaaaccacaataacttttttctgcaaagtaatcaactgctaagagcgtacgttctgtcacttattactggtaaagtttagaaacttcaagcctggccagagtgaaacaaacaaaaaggtcgaaacgcctgcagttggccggccgtggtggctcacgccttgtaatcccaacactttgggaggccgaggcgggcggatcaccagaggtcgggagttctcgaccagcctaactaacacggtgaaaccccgtctctactaaaaatacaaaaaaattagccaggcctgggcgcaagctactggggaggctgagacaagagaatcgtttgaaccctggaggtggaggttgcagtgagccgagatcgccccactgcactccaacctcggcgacagagtgagacttcgaatcaagaagaaaaaaaaaaaaaaagaacgaaacgcctgcagttggttagagtgcctttgtttaagaactctttatattttgctgcacaaaggaaatggtgctccagaggcaagctgtaaaagtcacatggtcgtagaggaggtagataggtagccttgtcctggaggcacagtgcagggtccagttgcagtctctaggggcgggaagccggggctttccttcactcacctgctcccttcctcttcttcctccaccgcactccccagtgcatgttctaggcctgatggaggttactctacatagggttactaaatacattgttgatggagccgatgggtagattttcatagagagtgcagtgaatgaagacgatgacagtagaatgaccgaagtcgtttgggaagttgaacaaagtttagaaaggtagccggtctcgggtgggaggggctgcgtggggatgggcctacagagactttggaatctgtaggtgggctggctttcaatcccagcttcactttgccgaccttggcacgcctggcccactttctctgtccctgttcctgagctgtggagctgtgagataaggtatctaaggcatcaggccatcaacaaatggtagctggcaggattatctggagggactatcaaattagagtgaggtgaaaaaaggaatgattagttttgtaaaacctaagctgatttaaggaataaggaggtggcagttacattatcctgcaaaggaagagaagtgaaaagagggccagacacagtggtgggcgcctctagtcccagctactggggagactgaggcaggagaatcgcttgaaacctggaggtggacttgcagtgagccaagattgcgccactgcactgcagcctgggcgacagagggaggctctgtctcaaaaaaaaaaagtgaaaagaggtcaagaatcaggtttaaagaagggcacatttttgataagtagattgcagatgaaaaggaatttctatatctgttatacagggtagctccttcattaaaatttattttctccatatcaataaagctatttcttactttatccaggtttttaaatattaacgcaggccgggcgtggtggctcatgcttgtaatcccagcacttccaggaggctgaggcaggtggatcacttgaggccaggagttcaagaccagccaggacaacatagcaaaaccctgtctctactgaaaatacaaaaaaaaagtagctgggtgtggtggcgcatgcctgtaatcccagctactcaggaggctgaggcgggagaatcgcttgaacccggaggcggaggttgtggtgagccgagaccacgccactgcactccagcctaggcaacagagcaagactccatcccaaaaagatagtaataaaaaataaatattaatgcggcataatgatacttaagtgcccccttctggaaagcttgtcttcttgggatacacagctcaaggtggtcatgatgtgttattttctctcaatctgtgggtaaagtttatcaactccagaaaagtattggaacttaggtcctggcatctgtctccatagctttttgttctgattcagtgtttgctagagtggccactggacctggtcattaactggtactgcaggatgcagggtgcactttgtgagagtgaaggtaagttacagctggccagcatcagaattgggatgtccagagcctaggtttttttctacatccgtagcattaacaaacaacaacaacaacaaaaaacttaaaaaaaaaatccctcactgaggttgccaaatctgttaactttgaaactgaatgtattaagagatggggtctagctctgttacccaggctggagtatggtgactattcacaggggcagtcatagcacactatgtcctagaactcttgggctcaagagatcctcctgccttggccttccaggtaaccgggactataggcttgccactatgcccggccagaacgtcattttaaacagattttcttgagcagctacttaatactagttagccattcctaaacctgtaggtcttgactaatgtgagggcatatttgagacaacccaaactcacatcttaagttattaattttaatgtgtcagctaacaactgggaagctacttctttggcaatatgagaaacaagagttttaaagacccaagcaagaatacatctccagtgttttcaccctgagggatatgagttagtttgctgagcagccatttggcacagcttccatgagttaggttgtgagccgccttcggctcagcagtcagtatggagcagaagccacaggaggtgagccgctggcctgctgccaccgaggaggacactgagacatgagtgctttccaagttgtgcagaattttggcatgtgaggaggatggttgtgacatatggatttaattttcttgaagttattgtttctttatactctaattggttaaaagataagcaggtgaggttatcaaaaggcagtagattccgacagcttttgaaaatgggaagagtgaaatccctgtgctgtcttcatagagcagtgtgtggagtaggaagtacctcgttactgtgctcacctagggcaaaggtcggattaaacagctttctcatctgccatgcagcggggaggaccagttggagaggttgcttttggctctcacaccttagtgtaaaaatcactttgtgaaaaatgcagctgtcacctccttcctccaagattctgattctgcctcagttaggatccaggaatctgcattttaacatgcacaccaggaattctgatgcccaaggttgagcaaaactgacttagggcgtacagacagaggatgccttagtgtggggtcttagttgggtctcattgcttggggcagttccaggaggaataggctaaaagggacttgaactgaattccccaccttgtattttttttgaggagaagtgaggttagatcaatcaggtgatgggggctagtaatggggagatctctaaggagaggggaaccaacccttttggctaggagatgatgggtaagagtccatgctttagggtcattcttcctcaactgaagggtgcatcttgctatactgctaatatttcagtttattcattgctgtctcccctctaagctgttgagctatcaggtaaaaacagatgcatgagtggatggggcctttattgtagaagatgccacactttgcactgtccagtgtagaagccactggccaattcatttaaattgacaataaaatcgagttcctcagttgcattagctgcatttaaatgctcagtagacacatgtggccagtggctaccttatgagacagtgcaaatacggaacaattccaccatcacagacacttccatgacatagcactgctctagagagtagagaggaattcagtggaaggctgtccagggagtagggagtccagttgagaatggagagcagagggctccccttacagggttggaaatgcagatgtgtcatgcaggagatccaaggtccaagtctttgcccttgcccttcccttcctccacccaaacttctacctcttccaggcttacaccactgctaatccagcattaaccacaaactatatttcagttttcctgtaggacttaattctttttcagaaaatgtgcaaaaacattttctgcataacaaagcaacttcatatttcaactccgtaatcacagaactgttactctaggcatattttgcagtgaatgcatttgtgtaatattttctccatttctgtttataagattgttattttgcaggtcaagttgggggtgagaaagcatagagctgttatagtttgtttattagtaagcagtttcccttcatttccttttcttcacatactttggcttttaaactaaccaaaacagaagggtcttaagagtttgggagtcagaagggggatgggagttcctactatattttactgccataagtaaaaaataattaacaaaaatcaactttttttaataggtggggaagaaaacatgacagaaacagatgccttctataaaaggtaaagctgtgctcttttctaataaaggtctcttcctttgatggtcatgatcagcctacagaaatcaaagcaagaatgggtcctgggagctaggaagtaaaactgagtaggccaagttttagcctttggggagctcatttgtagataggcaggttgtgctgcccctggttgtcgagggacccggagtgatgttcagaatgcatattaattggcatggcatgagcctagagctagaacttggttctgagcctcgtggttattcctggttgctggatcctgggggccaggggaaagacatcaaggcagagggttagggatgtgtcaggatggcacatggctgctctttaccaattggcagagagatatttcaggactttccttattagcatggcctttcctggagcacacccatggaacatcagcctggcaaggaggaagctcagatctctatctacatgttgacctagaacttggaggggctcattatttaatatgacttgaagtgattgtttttcaacttttcattaacagagaaatgtttgatccggcagaaaagtacaaaatggaccacaggaggagaggaattgctttaatcttcaatcatgagaggttcttttggcacttaacactgccagaaaggcggggcacctgcgcagatagagacaatcttacccgcaggtagtagttttatctacctatcaagatgaggcagttaagtgataaaattacttttggccaggtgtggtggttcatgctggtaatcccagcactttgggaagccaaggtgggaggatcacttaaggcaaggagtttgagacaagtctgggcaacagagtgagacctggtctctacaaaaaataaaaatcaattagctgggcatggtggcggtagtcccagctactctggaggctgaagggggaggattgcttgatcccaggagttcaaggctgcacggagctgtgatcatgccactgcactccaggctgggcaacagagtgagaccttgtctctaaaataaaaaataaaataatttttttccatgtttccaaatgaaaagttaaaaaaaattaaaaattaaaaaaattccagacttctagatgtagggaaaagttacttatcataacttgtcaacactaatgaggtaacaacagatcaagagtgttggagcatgggccttgctttgatgcagcctcccttaagaacttgcctgagttggccgggcatggtggctcactcctgtaatcccggcactttgggaggctgaggcgggtggatcataaggtcaggagattgagaccatcctggctaacatggtgaaaccctgtctctcctaaaaaatacaaaaaattagccgggcgtggtggtgggagcctgtagtcccagctactcgggaggctgaggcaggagaatggcatgaagccaggaggcggagcttgcagtgagccaagatcaagccactgcactccagcctgggcgacagagtgagcctctgtctcaaaaaaaaaaaaaaaaaaaaaacttgcctgagttattttccttactctgttctattaaatgttgacaggcttttgcatttgtactaacatttgcatagtagacaacaacactctaaattgaatcggctgtcattatgattaaatcctttccgaccatggtgaatgaagggaaaaccaccattgttatatttcatttcttttctcctaaagaaagcaaatttatgtgtcgttaaaatgtgtatagattcttccaaagtaagggtatttgacaactttgaaaagtttataggctaacatttcatttaatataccactaggttttcagatctaggatttgaagtgaaatgctttaatgatcttaaagcagaagaactactgctcaaaattcatgagggtaggtagttttgctatcatatagttcatcttcctctaatgaaaccgagtcctttatcctatctgtaaagacaggtacaagtaattacttgtgtgacaaaaaaagtattaatttagcacaggttacaggttttgggtatgctgctgtgtttccgttacccagcacatttagagacccttgcaaataattatgcctctaaagtttatatttagcataccactagttacagtctcttgtagaaatgaatgaaatgaccccttcaattggcagtaaaagcctaaaatgcaagtactacaggtgctgggcagtcaacaggctcattttctcattgactcctttcatgcctcattttctcactggtttccaaatagtgttggtttacatacattacctcctttaattctcacagcgtcaatagggggtaggtattctaattataacaacctatattataatcatcgctgcatgccaaccattatatttgcataattactttaaactttcttcagagctcttctttaaactgcacatcattctaactttggctgcttttaccagcttttctactttgctcagaagctttggaggacatcagtctcatggcaccgcacctggctttgagtactcttttggaagctctaagtgctataatcttttttttttttttttgagatggagtctcgctgtccgccccaggctggagtgcagtggtgtgatctcagctcactgcagcctccacctcctgggttcaagcaattctcctgcctcagcttcccaggtagctcgcccccatgcccagctaatttttgtatttttagtagagatggggtttcaccgtgttggccaggctggtcttgaacactggcctcaagtgatccacctgcctcggcctcccaaaatgctgggattccaggcgtgagccactgcgcctggccctaagtgctgtattcctatagtgaaagtgcagcaatgcgtggggagctgttgagcagcagtttctgggtctttccactctgctctaaggtctctgctgattcaaggaaatttttagtttattgccaaaataacttggatcttaagtgagattccctatcgtaatccctttatgttttccacatcattctgtgttttagaaaggctaatgaaatttggtcaaactctattactctgaactttaataaatttggatttttttaaaaaaataagatacagtacggtatattgtttgattaaagactaagttgtttcatcacagatattttacacttattaaagagtaggccgggcatggtggctcatgcctgtaatcccagcactttgggaggctgaggcgaggtggatcacctgaggtcaggagttcgagactagcctggctacatggcaaaaccccgtctctactaaaaatacaaaaattagctggatatggtttcaaatctgtaatcccagctacttgggaggctgaggcaggagaatcgcttgaacccgggaggtggaggttgcagtgagccaagatcgcgccactgcactccagccctgggcaccaagagtgaaactctgtctcaaaaaaacaaaaaagacaaaaccacaaattttcctcctccccaatcatgtggatgtaactttattcttgtgttaataaactgaatttttatgtgtagataacaagcatcatattttcatttctatattctcttttgggtcctttatagtgtcaactgttagccacgcagatgccgattgctttgtgtgtgtcttcctgagccatggcgaaggcaatcacatttatgcatatgatgctaaaatcgaaattcagacattaactggcttgttcaaaggagacaagtgtcacagcctggttggaaaacccaagatatttatcattcaggtaagactgagtacattatcatcttaattataaagtctgtcattctgaacatgtgataaaggtctaaaagccagttttgaaagtaactttctccagtaattggacaggccgtttttgaattctggccaatcatagttgcactttgagctgttagtaataattctttgacactctcattgtctatattacaaaataacagcatttggattgagatttgaagtgactcacaaagtaaccaatgtgctactccctgtccgagcctgataccaaggagagccattggaaagcgtacctgctgattgaagagtgccgcacaaggcaacctcatcagactaggtttgcttctttgggaggtggagagcatataaaccagtggaaagaatggaaaaataaagccttctagtcttatcctgtctcatccctttcacattgccccttaaaccaagattgtttccccgctgccaaattggttttagggataaaagtattttttacaggcgtgatggctcacacctgtaatcccagcactttgggaggccgaggcaggcagatcacctgaggagttcaagaccagcctggcaaacatggtgaaaccctgtctctactaaaaatacaaaaattagctgggcatggtggcaggtgcctgtaatcccagccattcgggaagctgaggcatgagaattgcttgaacctgggaggcagaggctgtagttagccgagagcataccactgcactccagcctgggcgtcagagcgagactctgtctaaaaaaaaaaaaaaatttttttttttgcatgaatattacttggcacatcattaacgattttttttttttttttgagatggagtctcgctctgtcccccaggctggagtgcaatggtgcaatctcggctcactgcaacctccgcccccaggttcaattattctcccacttcagcctcccgagtagctgggattacaggcacctgccatcatggctggctaatttttgtattttcatagagatggggtttcaccatgttggctagtgtggtcttgaactcctgacctcaggtgatctgcccgccttggcctcccagagtgttgggattacaggcgtgagccactgtgcccggccaaagattttgaactacaaaagagaaacctgacatgccagtgtcacaattctgtatttccatgaatagcatggaggacatgaaataacattatgttagtctattcttgcattgctataaagacctgagactgggtaatttataaggaagagagatttgattggcctacagttctgcaggctgtacaggaagcatggtgctggcatctgcttctggtgaggccttaggaagcttacaattatggtggaaggtgacagagagccagcatatgacttgttgggagtgggagcaagagagtgccaagggagaggtcccagactctcttaagtaaccagatttcatgtgaactgagtgggagctcacttacaccaagaggatgatgctaaaccattcatgagagaccctccccgatgatccagtcaccttccaccaggcttcacctcccacactgggaaccacattttaacatgagatttggaggggacagacgttcaaaccgtatcaaacatgaaactgcatttcgcatcctaccataaagccttgttttggggtgaggaagtggctgtaagtgaagaatgactgcctcgtttctggtttggacaactgcattggaatgtctgtgggacatggaattggtcatacaggtgtagaacctaagacaggtggcctcggctggagaaataaatcaggaggtggtcgtggaagccatgggattagatgagtttactcgtgttgaatacagcctgaaaaaggagcataaaacaaaaccgaggggcacccatgaggaatgggctgaagagggaagatactgaggaggatggacaccaggagagaaacaagaaacaaataatgacatgattgttttgtgaaaaggggagtggccaacactgtcaaacacaagagaattggaaataactaatgagctactgtgggaagaaagccacaaatgaaatcgttttccaagttactttctctctctatgccttagattcttggtaaaaagagcacctgcctcatggggtggttgtgtgggcaaaatcacctgactgatttaatgaatgcaaaccacttagaacagtgcctggcatacagcacgccctcagtaaaggctcgctcttactcttattgcagttgactgggcaggtgatgtcattaggttaaaggtgttctgttatatttaggaaggacagtagaaaaaaggcctgaaaataagcctgctgatacgaagattaaaatctgcctataaaaacgggatagtagggattttctatttattttatgatttgaaatcattcaggactttcttttgatgctcataattctttaaccatttcagaactgattgatgcatttataatgctacaacagaatcttattggctggctgtattcgtgtatagggtgaccataacaaagtaccacagactgtgtggtttaaacagcaggaatttatcgtcacagtcctggagctggaagtctcagatgaacgtgtggggaggattggtttcttctgaggcctccctttttggcttgtaggtggccatcttgtccctgtgccttcatatggtctttcttctgtgcatgtctgtgtcccaatttcttcttatgaagacactagtcatgttggatttggtttcacccgtatgacctcattttccttaatcacctctaaaggccctgtcttccaatacagtcagagttgaggtattggggctaggacttcaacatatgaatttttggggaacacaattcagcttgtaaaagtcacttatgagaagagtttaatgttagcctctgagtgagtcagaatttaatgttaattatgagtctaaatttaatgtaaattctgagtgagtcagaattagtgggtaaaaatttgagacctcactttttaatgggcacacaaggaagatggtgcattagtaggtatctcctcaggtcaggctggatgtcattttaactcctaacaatttatagcagcagccaggcaagcaaaagtcaatctggaaatacactgcctgcctaaacagaaacacttaggtcaatactatagctttcaaaagggaaggtaacaccgtgtgccctgggtgtggttaatctgtaagaaattttaaaaccacaagctttttcctctacagtatacccttctaccaacataatttgatgtatacttggggatgccaattttttcttaaaacattaactaaaagaaagcctaagtaaggaaacaactttgttggcttttgtgccctccaaacctgaatgtattgcttctgaactgctcagaatgtggtttctgttgtaatgaaggggtactggcttacacattcactgcacacattaagcactgggaaggaaacagtaggcaaagaggtgggtttttcctccttgtcttgtaggcatgtcggggaaaccagcacgatgtgccagtcattcctttggatgtagtagataatcagacagagaagttggacaccaacataactgaggtggatgcagcctccgtttacacgctgcctgctggagctgacttcctcatgtgttactctgttgcagaaggtgtgtggtgtttaaaacaaacatataattgcctctcttggctaaggtcctgtaagtttcacctttgattggtttgggtcagatctttcggttgggacatggtgattccatcctgaagctcacagacttagaatttctttgaccacatggcacagagtgataaatgaaagtgtaaagtaccttacagaaaaataattcctgagagttatgttgagtaaagcaacagtctaattaattatattagtattagcagcctaccatcagttactgagggtggtagtgccattgtggtaattgtcatggaaagctttaattttttttttttttgagacaaggtctcaactctgtcactgaggctgaagtgcaatggcatgatcatggctcactgcagcctcaatttctcaggctcaagcaatcctcccacctcagcccccctgtagctgggactataggcatgcaccaccatgcttggctaactttttaatttttcggtagagattgggtcttgctatgttgcccaggctggtctcaaacaatcctccagccttggcctcccaaagtgctgggagtacaggtatgagccattgcgtgcgtgcgtgcgttttttttttttatagtctcactctgttgcccagacgtgtgtgtgtgtgtgtatttttttttttttttaatagtctcactctgttgcccaggcgggagtgcagtggtccaatgtcagctcactgcaacctctgcctcctgggttcaagtgattctcctacctcagcctcccgagtagctgggaatacaggtgcctgccaccacgccaagctaatttttgtagttttagtagagatggggtttcaccatgttggccaggctggtctcgaactcctgacctcaagtgatctgcctgcctcagcctcccaaagtgctgggattataggcatgagccaccatgcccaactgaaaaatattttgataatggttctttgcttttgtagtaaataattttgacttctttttagtaatctcatgtctcaatcattgtctatcttgggaattttttaggaaatggaagaaattatacagaattaaccccgtgtatataaaactatatagaattaatctcttttcccatttgaaaagagtcaaaaaaatttaaaagccatttatatcatggacacatcttaagactcttctaaggatttttaagaacttatacttctgtaactaaaaagaatacacacagtaattgaaagccagaaaactgcagcaacattttccttctcacttttttaggatattattctcaccgggaaactgtgaacggctcatggtacattcaagatttgtgtgagatgttgggaaaatatggctcctccttagagttcacagaactcctcacactggtgaacaggaaagtttctcagcgccgagtggacttttgcaaagacccaagtgcaattggaaagaagcaggttccctgttttgcctcaatgctaactaaaaagctgcatttctttccaaaatctaattaattaatagaggctatctaattttacactctgtattgaaaatggctttctcagccaggcgtggttactcacacctgtaatcccagcactttgggagtccaaggtgggcggatcacctgaggtcgggagttcgagaccagcctgaccaacatggagaagccccgtctctactaaaaatgcaaaaaaaaatttagctaggcatggcggcgcatgcctgcaatcccagctacttggaaggctgaggcaggagaatcacttgaacccaggaggtggaggctgcggtgagccgagattgcgccattgcactccagcctgggcaacgagtgaaactccgtctcaaaaaaaagaaaatgtctttctcttccttttatataaatatcgttagggtgaagcattatggtctaatgattcaaatgttttaaagtttaatgcctagcagagaactgccttaaaaaaaaaaaaaaaaagttcatgttggccatggtgaaagggtttgatatggagaaacaaaatcctcaggaaattagataaataaaaatttataagcatttgtattattttttaataaactgcagggttacacaaaaatctagctgatttaacttgtattttgtcacttttttataaaagtttattgtttgatgtttttaaaggtttttgaaatccaggaattaaatcatcccttaataaaatattcgaaattcatattttattacatttttctaactcacattcttgttcatttgtgtaattgatagatgttttttactcggtcatttaaatgtttaatatttcatctacatatgaattaccacattttgtgtatgactttaattcctactttaaaaaaaatcagcattccagaattgttgtatggcattctgaggatgaaatagaaaacataattcttagccactataggttgaacacctgcaaaatctgattctgtcagcatcttccatgttaagttcctacaaacagtccaacagtcatcagacccattcacacacaatcacattaatagaataacagatgttactttcaaggttcaattcttattaatatgatatagaatctgaataatactgttttgttaataatctgccagaggctgaagaaagttaactgttttggcttcgatacctgaaacttgtgatttgcaggttcaaaagcaatcgtttatatacactgtttcagttgaagttcaaaaagtaccaactatactttatggttggtggaacatttatgcctttttgataaatgataaaaagatcttactcccaactactgcatattaaaaaaagaagttaatgacaagacgattccgtgggaggttgaggcaggagggttgcttgagcccaggagtttgatttggcaagacctggtctctgatttttttaaaaggatgattccaaattaagtctaagaatggaagtgaatgccaagcacagaccctttcacctccacatagagaataaacgtgggacaagctgacaaggatttttagctaacagcagccaccgttgctttgatagacgcatacaagccctttctaacattagcaagtcataaaaatccaaaaagtaaaaatctcagttgagaacttcaaacaatacttctgttttacaagaatttaataacaaagattaaacaatcaaaatgaaccatgggactcaaaaacatcctaagttgcaaaatcaatacataatggaaaatctgacgacatttaaaactgtaagattaattcttttcattgagttcttctacttgcatttgcaaacagagagaatgacgcgcctgtttcagggattctttagcctgtcatgggaaaaagaaaaagcatgtgatcagaaagaagagaaacataccaacattactgcttgagaggaattctataaacttagcgtctccttacagcaataaaatgtttaaactattagtggcaaatggctgaaatatatatatatttttcttgagatggggtttcattctgtcacccaggcaggagtgcagtgctaccatctcagcccactgcaacctctgcctccctggtgcaagtcattctcctgcctcagcctcccaagtagctaggaccacaggtgcccaccaccatgcccacctaatattttttggtattttttgtagagatggagttttgccatgttgctctggctggtcttgaactcctggactcaagtgatttgcccaccttggcttcccaaagtgacgggatataaatatttcaattttatatgtggatctttttaaaagatgagtttaagtttactaattcatccttatgccttaaaaaaaaatacagtttataccctttataatggttttccacttaatattcttaaaaaaaaaacccttaacctaccactttatctttttatctccactttattaccttaaacataattaacaaggtgttatatttaaatttttttattttagagatggggtcttgctctgttgcccaggctggagtgcagtggtgtgatcatagttcactgcagctttggaactcttgggttcaagggatcttcctgcttcagcttcccaagtagctagggctacaagcatgtgccaccatgtccagctaatttaaaaacaaattttgtagagatgaggtctcactatgttgactagactggtctccaatttctggactcgagatcctcccacctcagcctcccaaatcactgggatcacactaggcttaccttctaaatctcaaatgcactgtcttgtaaactagctgaaagaacattccttagccttctcagataggattattcaaatataagggaatccacaaattttttttaaaagcattatatttttcctatagaatgactatatgaaataaaatacattttttaaaaaggcttggctggacatggtggctcacacctgtaatcctaacactttgggaggccaaggcaggtggatcacttgaggttaggagtttgagaccagcctggacaacatagtggaactctgtctctactaaaaatacaaaaagtaaccaggtgtggtggcgggcgcctataatcacagctaactcaggaggctgaggcacaagaatcacttgaacccaggaggcggaggttgcagtgaactgagattgcaccactacactctagtgtgggcaacagcaagaatctgtctcaaaaaaatttaaattaaaacgtccttcaatttttggactacaaaatttgagtgattctgttttaaattctagggatgagtaatttagaaagtacacataacaaaaggtttgattaccagctactaaacaagcatgcattgagcaatggaggatatttttaccaaacttttttgttatgaaattactgataaaaatgaaaaatccagtgatgtgctgagaagtcaccatctgaacaaagttaatgagtaaattgggaggccagagataactggctaacaagcagactgtgaaaaagtacttaagaatatagactccgaaatcaaacttgtgttcgtaccatggttccttcactttttagcagtgtatacctgagtaagtgcctttcagaagggaaaaagatgaagggaggaatactataatgattcttctatctttagggacaagaaacttataaatgccttaagttccttatcacagtgttcaccttcctaaaatgctattttttattatttgcttagaaatggatttccagtgaaaacttttattttactatctcaagtcatttaagaaattttaacacacatatttaaagctatcttcatgtacagaagagtaaaactgggatatgaaattaatatgaaatatgtcaaccatttcaatgaacaatcccacaacacacagaaatctaggaccaagatttaatttgtgaggatgagccaccagttatacttccctgatactaagttctggctcccaaacaaaccagctataagatgtatttgtagtataccttagcaaggtcttcttttaacttgttgtattgctgcacatcaaagtgctgttgctttgatttcttgtggaagaactgaagaaattgcctactcttaacagctgagtaagtataatccttaaaaaaaaagagagagagaagaaaacaacatgttttgaatgaacataatgcctctgcaagaaagatacttgactcagcaacaccaaagcaaaagaaggcagatgaatgagtgaaatatttgcaatggccatgagagtaagcataagccaacacaaaacatttagtctcatttaagatctgctgctcctttcaaaaacaggcaagctgggaagagaaattacacataatgggaaagaagaaggaagaaataagaaaagggagatttttggcatattaaataaccatgaggcaaagctgatgttactgaatgttttgcaatgcaaattcagttttttttataaatgagggcacagagaacacatatttatcccaatggaggaatttgtgtgttacactaattacttctccaaagaagttatatataatttctctgtgttgtgagtcttcattcagtcttgtgaaaaacaataaaaaatttaccagctcataaaaaccagatggcagtaattcagtaaataaaaaggcttttttgctcattgcttagataagacattctttgcaaagatgttctaacttaccattttcctaacaaactactcacctgtcgagtgactataaagtatgcaaaaaagaccatagaatttgcaaatgtgatgaagtatgtaactggctccatgatatcccaggagtacacccaccacgtgagccaggccagtgccccaccctgaatggacagcagtgccaatccagcccacaggagtccactggttttggcttccgaatgagcttctattccagctttcacctgagggggaatgaaaaactgttaattctgtttacaaatacaccaactaaagggacaaaaagcaagatgaaaaggaactcttgccgggcgcggtggctcacgcctgtaatctcagcactctgggaggccgaggtgggcggatcacgaggtcaggagatcgagaccatcctggttaacatggtgaaaccccgtctctactaaaaatacaaaaaattagccgggcatggtggcaggcgcctgtagtcccagatactcaggaggctgaggcagaagaatggcgtgaacccaggaggtggagcttgcagtgagccaagatcactccactgcactccagcctgggcgacagtgcaagactctgcgtcaaaaaaaaaaaaaaagaaaagaggaactcttgaacaaatgcacatctgcattactttgaagaggtcagacatatggaaaaacaatttatgaaacatacatgaatgagtaaatgctcatttcaaatcagctcactaataaaactctttaaaactataaaggtaataactgcccttggaaaaatgaatagtaaagtatacaattgaaaactcagaagtcatcctctgaaacccaactaaaattagaggcctatttgactcagcaattctacttctaggaattcattctgaatatatacctgcatacacaaaacccattacagattcattctcaatagctgaagtcctaaaagaatccatatgcccatcaagatattacacaactgtaatgaagctctgtgccaataaggagagatttctataatacactgaaaaaagatgagaatagtgcataatagtgtgcttccttttctgagaaagagaataaaataagaatgtatttgtttgtacacagataaaggaaatctgaaatgattcataagtgtggagaatatagtaactgtgtggaggacagggtgggaggaaaactttagattttaaaattttaatccaagtaaatatattacaagtaaagaaaaaactaaaatggtaaacgataaaaaaaaccaaacaagtccctctctctgccatcctccaaatacccttagaggtaatcactattaaaacttccttacatctccagaaactttccattcatattcaaagatacacatataccttcccatttaaataaaaaccacaaatgggattataagtctaaagaatgcaattctttcccttgaaacaaaccaatttccccaactatccccccaaatcaaagttattttttccacaccattgattaactttagatgctagccattaccaactttccagctgcaattctgttaggcacagcaggggctctgggcctattctctaaaaaggaaagggaggtaggactccagctagtgctgccctgctcatattatacttatttacagagacattatgactgatctctggagaggaggcatggcatagagtttaagatggcagctctgaacacaggccacgtgagctcaagtccaggctctaccatgtattagcgatacagctctgttgctcagtttcctcatctgaaaaatgaggaagataaaagcactgaacttacaggttttttgtgaggcgtaaaggagaaaattagcatatggaaagcattcagaaaaatgctcacatgaggagcactcaataaacactaggtattattttcaaatatactcaaccgtgatgcttcctaacctgttcaaggggctgcagctgttccttcaggtggtcaattttctccagtaaatggtgctctctctttttctgagactcttctaaatgcaagattgtaaatagtctgtgaaccaaagatttcatgtgttccatctcagcagtgtgctcattacttggtttttctgagaaaagacacaagggaaacaagccagtcaccacattgttcccgcagggtgtgtaaatctttcggtccccaattctctttccaaatattatcttaggatccacagggtaaggaagctggtgtacaaacatgtaaacaaaggtgtaatatacatttgaaaagcagttcaagtgtacttttcaacaaagagagtcacagtaaacaacaaagtgtggatctgaagagcagttcatatacaaggtaaaaaaaaaaaaagtcaccaaaaaaatgactttggcataaggattattttcagctgaaggcaattgagcataagcagctacaagaaaggctctctggcctctccctttttgcccaaaagcaggacacaaattttacaaagacctcgtgagagacagaactagctggatttcctaggccaactaagaatccctaagcctagctgggaaggtgaccgcatccacctttaaacacggggcttgcaacttggctcacacccaaccagagaactcactaaaatgctaattaggcaaaaacaggaggtaaagaaatagccgatcatctattgcctgagagcacagcgggaaggacaaggatcgggatataaaaccaggcattcgagccagcaatggcaaccccctttgggtcccctcccttcgtatgggagctctgttttcactctatttcacactattaaatcttgaaactgcactcttctggttcgtgtttgttactgctcgagctgagctttcgctcactgtccaccactgctgttttgccgccgtagcagactcgctgctgacttacattcttccggatccagcagggtgtccactgtgctcctgatccagcgaggcacccctttgccgctccagatcgggctaaaggcttgccatttgttcctgcacggctaagtgcctgggtttgtcctaattgagctgaacactagtcactgggttccatggttgtcttccgtgacccacgacttctaatagagctgtaacactgcatggcccaagattccgttccttagaatccgtgaggccaagaaccccaggtcagagaacacgaggcttgccatcatcttggaagtggcccacgaccatcttgggagttctgggagcaaggacccccggtaacactctctaccagaaagaacaaaggtagatcgccagagacaactttagacccttttcagcttggagactgcaccagaggaatctacagacaaactttagtagcttttgactacctttaaccaacttatttgcttttccacaatttgctgaccccagagactcaaggaccttttcctttgacttgtcacaactctaaaaatgtattattctttgtagaagatgctctatcagctgaggttcagagccacctctttgagaattactcattccttggatacctcccatgtgtctatggaatatacacattaataaactactgtttgttttcttcttagtaatctctcttatcacaggggtccatcccagctaagaactaagagtaaagggaaaattatttttcaactcttacaggagttacggtttcagagtttttaatataaagttctcttgattatagacttccctcatcccacctcctccatgggtttataagtccatataaaattaggctctgctatgagaagtccctcagattgatacccttctgagagctgtaattgttcattatcactgttaagagaacctgagggaggacaaggtgagaggaagggaagatggaccttcccaccaggcagcagcaaacaactgcagacaagagagacatatgtctagagttggtatcagcccagtaagttttccagtttgtgtcagtagtcctgaagttatttttcttccatcactacccaattcaagactgctgtggtctttacccccttccatagtctaggctatagctgccacagaggggtgaaagctaggtgttctcttttaggctttgagatctgtagaagagtcagagtccttttgttaatgaaaaatgtattggtacataaaacatatacatacagagaaaaggcatagatggaagataggcaaagctaggcactaaaacagcaagggtaaattttattaataactattgcaatagggtccagcgtgaactgaactcaacttcaatttgtgcagagatgactgttttaaagaaagaatgacagaatgggcagggtgaacagcaggggcttgaacagagtcagggaaatgaaaatttacaaaaagtggatagttggttaatgttactgggccatctgggtttgttaactggtacctatccagaagagaaataaacttctcataccttatgacaggatgcaagtcagagccaggcacccacgggttgggagtaagagccagagttatgtccttgaacgttaacatttcaaagagagactcccaagtccttgagaagacaggtctgggtgataaaaaatttataccctcaggccaggtgaggtggttcacgcctgtaataccagcactttgggaggccaaggtgggtggatcacctgggttcaggagtttgagatcagcctggccaacagggtgaaaccccgtctctactaaaaatacaaaaatcagctgggcatggtggtacacacctgtagtcccagctactcaggaggctgaggcaggagaattgcttgaacccagaaggcagaggttgcagtgagccgagattgcaccactgcactccagcctgggtgacacagagagactctgtctcaaaaacaacaacaacaacaacaacaacaacaaaattatacctcaaagcgggagagaaagttcataattgcaagctttctaaagtaactgcttgtctgagtccactagtccactttctgcttctataacacgataccacagaccccataatttacaaagaacaaaaatttgtttccccctctcccactccctctctacggtctcccagtctccctctcttgccgaggctggactgtactgccatgatctcggctcgctgcaacctccctgcctcggcctgctgagtgcctgggattccaggcacgtgctgccactcctgactggtttttgtatttttggtggagacggggtttcgccgtgttgaccgggctggtctccagctcctggcctcaggtgatctgcccgcctcggcctcccgaggtgctgggattgcagacggagtctcgctcactcagtgctcaatgttgcccaggctggagtgcagtggcgtgatctcggctcactacaacctccacctcccagccgcctgccttggcctcccaaagtgctaagattacagcctctgcctggccgccaccccgtctaggaagtaaggagtgtctctgccgggccacccatcgtctgggatgtgaggagtgcctctgcccagctgccccgtctgggaagtgaggagcacctctgcccggccatcccgtctgggaggaagtgaggagtgcctctgcccggctgcccctaatgggaagtgaggagcgcctctgcctggctgccccatctgggaagagaggagcgcctctgcccagctgccaccccatctaggaagtgaggactgtctctgcctggctgccccgaatgggaagtgaggagcgcctctgcccggccaccctgtctgggatgtgaggagcgcctctgcccggccgccaccccgtctgggaagtggggagtgtctctgcccggccgccccatctgggaggaagtgagagcgcctctgcccagctgccccgtctgggaattgaggagcgcctctgcccggccacccatcgtctgggaagtgaggagcacctctgcccggccgccccgtctgggatgtggggagcgcctctgcttggccaccaccccgtctgggaagtggggagtgcctctgcctggccgccccatctgagaggtgaggagcgcctctgcccggccgccaccccgtctgggaggtgaggagtgcctctgcctggccgccaccctgtctgggaggtgaggagctcctctgcccggccgccccgtctgggatgtgaggagcgcctctgcccggccaccgccccatctgggaaatggggagcgcctctgcccggccgccccgtctgggaggtgaggagcgtctctgcccgcccgcccatcatctgggaagtgaggagcgcctctgcctggccgccccgtgtgggaagtgaggagcgcctctgcccggccgccccacctgggaggtgtacccaacagctccgaagagacagcgaccatcgagaacaggccatgatgatgatggcggttttgtcgaatagaaaagggggaaatgtggggaaaagaaagagagatcagattgttactgtgtctgtgcagaaagaagtagacataggagactccattttgttctgtactaagaaaaattcttctgccttgggatgctgttaatctataaccttacccccaaccccgtgctctctgaaacatgtgctgtgtcaactcagggttaaatggattaagggcggtgcaagatgtgctttgttaaacagatgcctgaaggcagcatactcgttaagagtcatcaccactccctaatctcaagtacccagggacacaaacactgcagaaggccgcagggacctctgcctaggaaaaccagagacctttgttcacgtgtttatctgctgaccttctctccactattatcctatgaccctgccacatccccctctccgagaaacacccaagaatgatcaataaaaactaaaaaaaaaaaaaaaaaaaaaaaaaaatttgtttccttataaggaacagaaatttgttagtggaggctgggaaatccaagatcaatgcaccagcatctgacaagggccttcttgctgcatcataatgtagcagaaagcattacgtgggtgagagagagcaagcaggggctgaacttgtcctttattaaggaacccactgccaagataacacactcccatgataacgacattaatccactcacgttggtcctcatgacctaaacacctctaaaatgtcccacttcccaacagctttgcattagggatcaagtttctaacacatgaattttgagggacacattcaaaccatagcattctgccccggcccctcaaattcatgtccttttcccacacaaaatacattcactccatcccaagagccccaagcatctactcaaaagtccaattccaaagtctcatctaaatcagatatgggcaagaatctaggcatgattcatcctgaagcaaattatcctccagctgtgagtctgaaatcaaactcacagctggagtttgtacttccaaaatacaatggctggataggtatgggatagatatttccactgcaaaagggagaaataggaaaaaagaggtaactggtcccacgtaagtccaaaacccaacagggtagtaaacaacattaaatcttaaaacttcagaacaatctttgaattcatatgccaccttgtggatacactaaggcaggggttaggtcctcaaaatcttgggcagtcctgcccccatggctttgctgggctcagcccaggcagtagctcttatgggttgaggtttcatgcctgcagctctcccaggatgctgctgcacactggtagctctaaagttctggattctcaaggggcagctgtgaattttccaaatattttacattctgcctcatttttaattgtgaattctgtctttaaatctattctcctctcattttactgtatgcagttaaagaaaccatgcagattcttcaatattttgcttagaattttcttctgccaggtaaattagtttgtcactcttaaattctgccttccataaagtccttggatatgaaaacaattctttgtaactgcccagtcttgtgctattctgctattgcagcagaaaacagattaagacagaaaattggtactaggagcagagttactgctataacaaatacctgaaaaggtagaagtggctttggaatttggtaatgggcaaaggctggaagagtttggaagagcagactagaaaaagcctgtattgtcatgaacagagtattaagggcaattctggtgagggctcagaagttctttgcaagtccataacaataatggtgtttattccagcttccagtaagatattcctcatttcaaagagcttatcagattggcctttaccatatacatatttttggatttttagtagagacaaggtttcactatgttggccacactggtctcaaactcctgacctcaagtgatccacccgcctcagcctcctaaagtgccgggattataggtgcgagtcaccacacccagcctaccatcgatatttctacaaacattctgatcatgaccacttaagtaaatctctaaaaacttttagacttcctacagctattctcttccgagccctgaccagaattgtccttaacactctgttcatggcaatataggctttttctagtctgctcttccaaactcttccagcctttgcccattaccaaattccaaagccacttccaccttttcagatgtttcttatagccccacttctggtaccaattttctgtcttaatctgttttctgctgcaatagcagaatatcacagactgggcaacttaggaagaacaacattttatttcctcacagttctggagtctgggaagtccaagaccaaggtgttggcatatttggtgagacccttcttgctgcatcatcccatggcaaaagggaggcagagagaaagcaagtgtttatgcacaacagcacaagaaggggccaaactcatccttttataaggaacctactgccaagataacctctccagtgaaaatggcattcatctattcattagggcagtgcccccatgagccaaacacctcccaatacgcctcacatcccaacaccacaacactggggatcaagtttccaacacatcaactttgggaaacatactcaaagtacagcactgctctaaagatgaggttgtatggcttattgacctatcaccaggttttggctggaataaacagtaaattttccagagggtactgagctttctcagacagacattctaaaagaaccagagttgtcatccaaatgatgtggccttgagctatgagaaactatgcaaatgttggttcaactctcttagtgtgtgtggtgtgaggggggcagtgttgacaaaatctgtgctgagagtttacagtttttataggccatggttaacgcctagttgagaagagggttccgaggaacctgactgaagtttagtcaaggtgagagtctttgtcaagtggcacgaaatgtttatctgagttcaacaatgaatgcccttggctctgctctttcagaagcatgggcaccacagaattcatgttacaccaccaacctaaactgactcataacatttgtctgtggcatttcctaataaaggccaaaaatgagagagaattattcttttccttttcttttttttctttttgacatgggtcacttactttaataacaatacatataccatgttatcactatggaattaaattcagatgagacaagagaatttcacaagtgtgatagccttctgtatattatataaaagtttgggtatactgtctggccaaaccagcttgctcgtaagtcattaatcaaatccgttataggtaatttgttcagttcaatgtttacaattcttatgtaaaaaataagcaacacacacatttaaaaagtgttcatttacctttgcatgagtgcttaaaatacatatttctatttcaagatgacatttaaaaattattctaatatatcagcagcaaaaatataatttgcaattacaaaaaactaaactagaatccttaagttattctcatgtttacaattgtgattctttaataaatactaatattatgcagctctattgtttaagctttctggatttggtttaaacacatgcacatatattgtcaattgtgggaagctttacaaattatattccatgcactttttggacagagttctaacagagtcagccagtccacaaaacacgcagacaaaagttgaattaactggggcaaataggactcttatacaacatccaaaatatgtgagattctgcagcaaactgggagtaccttcaggattggcctgttatcttctttagaactaagttcatcttaaaaatttaagaaggtggacatttcaacaccatcaagtgcatttaggtgacatgtttaagttaacttgacttccttgaatgacggagttagtaaactagtcactagtaattcagtcaccaggcaaatcaagcctgcaagaaaggaagccagtattcaaaatgccatgttaccatctaaacccatacaaattagtttatttccaacaataataggtaacttcaataatgagacgtctaactaaagcaagctcctccaacaaggcagcatctcttcttctaaggcttaggttttgcccagaattcccgatacatggaatagcccataccaacagtcattgctcctacaacaaagccttgggctaccacacgcatgtggatcagatgaatggacattttagtatttcccctgctcttcagtttgtgtaatccatatgcaacaactgctgcaaaacctgctattccaatgggtacgaatggtgcctctttagcttttcgaataagtttggatccctgatattcctcatatgaaggaagagaaacacctgtgtctgctgacatagtgattgcttgaagaatctccctagagcgaaaaaacctctcacccgccaaccggcttctgatcttttccttttcttcagaagcttatttctcatttatgtgtcttccttgtgaacttaattttcataccccaaattttcaaatccacatttgcaatgtattaggaatattttagttaaatggagtttaaataacttttttgaaaactccaaatttgtaattttataaatttcattttcagttaatccagtatgattcctctgccattgttttccttctaactatacctgctgtgtccctgtttactttacactgtgagacttggcaagtctgatacagttttcataagctgaggttacagggaaacctgggctcactgacctcccatctccagggaggcttctgccctaggagttcaatcccaacacacagcccttccagcagcaagaggggttttctaatgagtgggcaggagtgcggaggttgtgctttatcccacttagacctaaggcttatggtccatgtggtggtttgagggatgggaaggtcatatggagtaatgggatggcctgtaaacatctatttccacttgtttgttcataaatgcaacccaaagaacagcaagtgaaagaccttccagggtgttaggaactcactggacatgaaagttgtataatatcttaacagggatttcctcttccatattttccaatttctgtcacccttctctctttgaagttaaatactgccctgatgttaaaagagaattcagaatggaagcaagtttgtaagtgttgaaatgagtgattcattttaatttcttttttcttgtttgtatttgaaaatcccgcataatgttcaaatatacaattaaaattttaaaaaattatttttgtttgaggggtcctcagagaaatggctgataaaagtgatgggacatgataggtacaaggtcaacctggatcatctttctatatcaaaaagtaaaagtacttacaaaaaaagggggagagggagcaagtcacaaagacacaggagtcactggtgttcaaactgtaccagataatggttagctggagctgcttcaagctggaggactttttttaacagaaatttcttatctaaattccctccctgttgatggctcttgcctgatctagaatttggctgcaaagtgatgattttccacctccagaaactctcccacatttaccagtagatgctcagcacctcactgtaagtaagagccttctattatttgttgatctatttcttttatcaccaatttattaaggcatagacttatgaatttcttcctcctacccttaacagtctataattatagcttactgggagaagaagcctcaggagccaatactagccagaattcttaaactgtaattggtgaattgctggacactcagtgtaaactagcattgaaggaaaaactcccacactttcatgagttttatctcctggaaccttaccaggttctcacagtgaagatcagagatcagagaaaaatactctccctgctttgggcaggagggaaggaaaaataaccattctgaaatagccctgagcacatgtcctccataataggaaactactttaccagagccttatctggcttggagaaagggcaattagtcaactacagacccctctagccttactgcctcacacaggagaataaaagaggctaagaaactcttgtaaatattacattttagagacacaggcccaattaaaaatatatatatatttaataataagattatagaatgattcctctcctcaatatcttaccactacattaacagggctccagttttatacaaggcacagattctatttgagaaagaatttatagggaaaccccaaaacatcaggagaaacaaaaataaggacactagaggaaagcctctgatacctacagctacagcaaacattaaacatctaactcctagccacataaaacctcacactaaagacctattcagctcagtttctattgcctgatacatcatgtctagctttcagcaaaaaatttaaagacaagctaaaaggcaaagaaaaaaacaacaacaaccacagtttgaagagacaaaacaagcatcagaaccaggctctgatatggcagagattttggaagtatcagagtaggatagtaaagcaactatgattaaaggctctaatggaaaaagtgcacaatatgcaagaacagatgagtaatgtaaatggagagatagaaacttagaaagaaccaaaaggaattgctagaaataaaaaccactgtaacagaaatgaagaatgcctttgatgggctcatcagtagactacacactggggaggaaagaatcagtcagcttgggggtatgccaacagaaactttctcaaactgaaggtcaaagagaaaaaagaatggaaaaaaaacctaacaaaatatctaagaactgtgggacaattgcacaagtgtaacttaggcataatgggactactagaagaagaaaaaagagagaagggaaaagcagcactatttgaactaataatggctgagaactgtccaaaagaatattgctaatggccaaaatggtaatttgagatgaaaaaagctctctttaagaaaaagttaagacttccagtttcctgtctgacataaaaaaactccaaagttgtcagtccatcctaacaattaataaatggctgaatacaatgaaaatcaacaacttttcttagatgcataagaaaactaaggttagagggaaaattgctgtcgccaaaattgaaaagatagataaatacagacaatgacaacttagcgaagcagagactcatgagcagaaatctgcaaagaatcagtaccagggtaggaaaacctgaactgtaactgacaaattgctggaggctcagcgtggacaactcttgaaatgtaagcattccagggagtcccataggtgggcgcattcccacacttttgtgagtttcacttctaggagctctactagatacagtgaaaactgaagaaaaatctcctcctccttacggcagcaggaaaggaaaagtagtgattttgaaatatgccagagaattctgttctttttctttttcttttttttttttggagacagagtctcctcgctctgttgcccaggctggagtgcaatgacatgatcttggctcactgcagcttccgccttccgggttcaagcgattctcctgcctcagcctcccgagtacctgtgattacaggcacctgcccccatgcccagctaatttttgtatttttagtagagacagggtttcaccatgttggcaggctgatctcaaactcctgacccagtgatccccactccctcagcctctcaaagtgctgggattaaaggcatgaaccactgcgcctggccaaattctgttactcttaacaaggactgtccacaaggtaaattattttcccagagcctaaccgattgggatttcattagagcttagttaacctaggggaaaagaaaaacccaacttcagcaacctgtaaccatcctatcccacctaaggtggtggggggtgggggaagggctgagaagcacttgtgaagttctaagtccaggggtacaggctcactaaaagactgagacctagtccccttaatcataggactacagaacacttcccctccataccttactttatcactatattataaaaggcttatttactgcagttccctttatccagtatatcacatccagcttttaacataaaattacaagacttactaaaaggcaaaaaacacagtttgaacagacagaataagcatcagaacgagagccagacatggcaggaacattggaattatcggactagcaacttgaaataactacaaataatatgctaaggaatctactagaaaaagtagataacatgcaagaacagatgggtaatgtaagaagacagatggaaattctaagaaagaatctaaataaaatgctagagataaagcattgtaatggaaaggaagaaggcctttaatgggcttgttagtagatgaaacatagctgaagaaagaatctctaagcttaaggatatttcattagaaactttcaaaactggtaagcaaagagaaaaaagactgaaaaaaaaaaaaccagaacagaacatctaagaactttaggacagccacaaaatgaataacatgtatgtaatgagaatactagaaggagaagaaaggaaagaacagaagaattatttgattaaggactgagaactccccccaaactaatgtcagatagcaaaccacagatccaggaagctcagagaacacaagcagaacaaatgccaagagaaaactacacatacgtatatcattttcaaactgcagaaaatcaaagataaagaaaataattcttaaagaagccagggaaggggggaacccttagctatagcagagaaaaaaaaagaaattaaacctgacttctctgaaatcatacaagcaagaagtgggataaaatatttagcattccgagaaaaaaacaaagcagcctaaaatttggtaccctgtgaaattattcttcaaaagtaaaggagaaacaaagatattttcaaacaaaaattgagtgaatttgttgctagcaggcctgccttgcaagaaatattaaaagaagttcttcagagaaaagcaagatgatataggttggaaatttagctctacagaaagaaagagaatcagagaaggaatagtggaggtaaaataaaaacctttatttttattattcttaattgatctactagataacagtcggttcaaaagaataatgctaacactggttttgactatgtatgcttagtatacctatgcttatgaatcactgaaatgaatgacagccgtgagacaaggggcatgagggagaaatcaggattattttgttattataaggtatttgcactagccgtgaagcaatataatgttatttgaaagtggacttgcaatagttgtaaatgtatattgcaaactcttaggcaaccgctaaaaaaagaagtataaataatatgctggaaaaggagagaaaatgaaatatacagaatgctcaattaaaaaccaaaaaaggcagaaaaaatgtggaagacaaaaacaggaaaaaaaaaaacagtggcaacaaatagaaaacagtagcaaatatggtagatattatgtatatcagtaatcaatttgaatgtcaatggcctaaatgaaccaatcaaaagatagagcttgtcagagtgggtcaaaaacccagatccaactatatattgtctataagaaacccactttaaatatgaagacatatatagatgaaaagcaaagggatgaagaaagatatactatgctaacactaataaaaagaaagctgaaatagctatattaatttcagacaaaacagacattagagcaaggaaagctaccaggagtaaagagagacattacataataataaaaggccaattctccaagaagacagaacaatgcttaatgtgtatgctcctaacacagcatcaaactacatgaggcaaaacaaaaactaatagaactacaagaagaaatagatgaattcatgtttacagttacagacttcaacattcctctattagaaatggacagatctgccaagcagaaaatcacggggtatacagttgaactcaatagcaccaccaatcaactgagataaacgtgacatctatagactatccaacaacagcacaatacacattctcaagttcacagggaacatctcatcaagacagatcacaatgaaggctataaaactcacatgagtaaatgtaaaaaaagaaaaatacaatgtatgctttcggatctcagtggaattaaactaaaaatcaataacccaaagatagctggaaaatcccaaaatacttagatattaaataatgtacttctaacacatgggtcaaagaagaaatcacaagaaaaaaattaaaatattttaaaataaatgaaaatacaacttatcaaaatttatggaaggcagtgaaagcaatgctcagaggggaatctataggattgaatacatataacaggaaagaataaacatctataagatctgaacttatactttaagaaactagagaaagaggagcaaatgaaatgaaaagcaagcagaagaaaagagataataaatatagtagaatatttattatgtagagtatataatatactctacataatatatattctattctatattataaacatagagtagaaaccaatgaaattgagaacaggaaacccacagagaaaaatcaatgatgttaaaagctagttctttggaaaaaatcaattaaattgataaacttctagccaggttaatcaagaaaaaaagagacaacaagttactcatggtagaaatgaaatgagaggatttaactacagatcccatggacattaaaaggaaaataaagaaatactatgaacaactctatgcccacaaattcaataacctagacaaaatagattaattctctaaaaggtacaatctatcaaaactcacacaaaaagaaatacacaatctacataagcctatacctattaagcaactgaatcaataataaatttccaaaacagaaagcaccaggtccagatggattttcttggtgaattctaccaaacatcaaaggaagaaactatattaattatctacaatctctttcagaagacagaagcagagggaatacttcctaactcattctatgaggccagcattaccttaatactaaaaccagacaaaaacactgtaagaaaacaaaaccacagaccaatatctctcgtgaatatagacacaaaaatcctcagcaaaataatagcaaataaaatacaacaatgtctaaaaagattgctacaccacaactaagtgggatttatccaggtatgcaaggctcaacacttgaatatgaattcatgtaatccatcatatcaacagactaaggaagaaaaatcacatgataatatcaatagatgcagaaaaagatttgacaaaattgaacaccaattcacaatgaagactctcagcaaaccaggaacagagggcaatttcctcaacttgtgaaagacaatgagaaagcaagccatagcctgggagaaaatatttgcaaaagacgtatctgatacaagaccgttatcccaaacatacaaagaactcaaaagaaaatgaacaacctgatcacaaaatgggcaaagatctgaaaagacacctcaacaaagaagacatagatggcaaataggcgtatgaagagatgctcatcatcatacatcattaagaaactacaaattaaaacaatgactacacacctactacaatggtgaaaatcacatccaaatcactaataccaccaattgctggcaaggatatggggcaacaggaattctcattcactgctggtaggaacacaaaatggtacagccactttggaagacagcttgacagttttacacaaaactaaacactctcaccatatgtgccagcaatcatgttcctgggtatttaaccaaatgatttgaaaatctacgtgcaaacaaaagcctgcgcatgtgtatttatagcaactccattcataattgccaaaacttggaagtatcccagatgtccttcagtaggtgaataaactgtggtacatacagataatgaacaattattcagcactaaaaggaaatgagctaacaagctctgaaacacatgaaagaaacttaaatgcatattactaagtgaaataagccaagctggccttcctcttcactttcttctttactgcctcgtctcctcccttatcactcccttttcttctctttgctctctcttcctttcccccttcttctttcccctcttgcctttctttttttcctcccactctcccccttttttcactcctctctctcactgtttagtattttccctgttgtaccccctttcttaaatatcctattttctcttcccttgctctctcatgtccctggccctactaccatgcgcccccacccctgccaaaaagaaagaaagaagcgaatctgaaaaggctatacaatcagcccttcttatttgtgggttctgcatctgtgaactcaaccaaccatggattgaaaatatttgggaaaaaaatctaataaaaattaacaatacaacaacaacaaaatgaatgaaaaaacaatatagtataacaaccatttacacagcatttacactgtcttaggtattgtaagtaatctagagatttaaagtatatgaagggagtatggaggttaaatgcaaatataacaaacattttgtatcagggccttgagcatccttgaattttggtagagtttggtagagtttgcctggccctattctgtataattcttaatgtcatagacaggttcttgaaaactgcaactttaaatgaaacaactataaggaaaccaattttaccataggctaattgatataagcaagagttaggttcttacagcatatttctggtcataaaaacatcattaaacatctaaataaagacccaagacacttttaatgttaaatattaaaataaatgtaagctacatatacatttaagaaagattaataaaaacaagatgattatttacattatgtacattaatgatgaatacatgtagttctacatttgtcacaatctatagaatatacaacaccaagagtaaaccctaatgtaaactatggactttgggtgacaatgatgtgtcagtgtgttcaccaattttaataaatataccagtctggtgtgggatattgacaataaggggcgtggggtgcagagagcatatgggaactctctctacattctgctcgattttgctttgaacctaaaactgctttaaataatacaattttttttttttacaagagttaagagctatacaatcatgtatacttgaattcttgattttcaggcctacctctctcttaacccaataatgtcggttaagcattcaagatcctaaagtaaaatcctgaaagtctaaaaagagtatacgggactaaaagtcctgctaggcaactgtaggtgatcacttatacccctgctggaaaagcttaaacactggttttccaaagacttcttggtcatctgataaatttaagattcttaacataaactttgctccctctggaaacctcacagctaaaggtaaaagtgaagataccagcctggacaacatagggagacccaatccctaaaaaaatgttaaaaaattagccaggcatggtggtgcatgcaggtagtcccagctactctggaggctgaggtgggaggattgtttgagcccaggagtttgagattacagtgagctatgacgacgtcactgtactccagcctgggaaacagagcaagtgagaattcatctaaaaaaaaaaaaaaaaagtaatagttggatagctgtgtttcttacctctctttggacactgcacatcatatgctattttattaatgacaagtttaaaatcattcattagcaaaatatccatcaaggtagaagctgaaatcatgttgccatctgaaagaaaaacatcatgcatgagcatgtctttgttttagaacagtaagaaccattttccaacacatagaaaattactaacccaggtctttacaatataatagtggacaatttacttttgtggggggtgtccccaaacccaaacactattcccattgaggggaatcctcctctattgctctacctcccatttccttggacgaaatggaacaggcaggcactccctctcccaccctgtcagctgtgctggaatccaggtagtgcccttggtgaaactgcatctgtggagagtcatgcaaaggttcaggggaggtggaaacccttcacagcaaggccagagcagtggcaggtgacagttttgggggtagggtggagtgtaggcagcagagctcatcaggtggttactgggttaacctggtctcagctcctgtcttctcttagttcctgccctcttctgggcctgtcttctgccacttccagcatgagagcctgttgacatactcctctgcttagccagagtccattctgctgcttatggccaagcttctgactggcacatgtaggcataagggtataactatttaaaaaccctgcctttctcttatttttaattattagaggtaactgatttgtggtggttaaaaatttagtagctaaatagcaccacaagctatcaccacatgggctcatcttcctttcaagcttgaaaaaaatgaagaggcaaactgtcaaggaggtgaggacaataatgaacgtgatgcctaagaaataaatttgcattgtatggtttattttgtggaaggggagagaggcaggagagattctaacagtaatttcaaatatcctaggagtatttcaggaaaaaaaaagtcttgaaaaattttaaccacaatgtgctttgcacagcctctcatagagcctaggttttgttatatttttgaaccaaactttaatgtaatccaggtgttaagaaagcaaattcactgaacaataagcaaaaaattggtgctgggtagaggtttctttttctatcattttcccttcagaatagttcacatataattacatgaaattaagtccaggaaaatgctggatgtgtattttcaaatcccggcctgacattcatttggttacattcaacccgagctattttagctatattaatatgtgtgatgacaggactaacactagtaacatcccgaagaaactggctgctgcctcttgtctgcctccctatagttcttttgttctggaagactgcaaatcaagtctgcacctgtgtctgttcctagaatttggctgtttgggttgctgttcatcagttgcagtgagatcattttccaaaaaattgccaaataattcaacccacacaatgggcaaaactgtgacaagcttagaacactcttttcaagcccaaattaaatgcaggcaaattccacccacagagtcttttaaaaagaagaaaaaaaaaatcagcaacacaccaatcgctttatctttccaagtgtcatttaaaaatttacatgtaaatgacttcaaaccagctccttaaaatcttactaaaaaagacatacactgaatatgatttctcagttgaaatactatattatcctttatacaaaggtatgaatagcaaaagactgacgtgaaacatttttaagcactagtgatacagtgttaaatttttcatgtattcttgggtatcaatttaaacccccgatagggtgtttgcactcagtccaaagagtcccttttctctaagacttctattctctttctttgtgctttctgtttcaaagctactaactttatttttagaaataagcaggataaacatctatcattttaacctctcttatttcattcacttcaatactaattgagtacacaccaccaggcaagt

SEQ ID NO: 58 - protein, 293 aa, Homo sapiens; identical to SEQ ID NO: 53

SEQ ID NO: 59 - protein, 204 aa, Homo sapiens
MSSASGLRRGHPAVSTVSHADADCFVCVFLSHGEGNHIYAYDAKIEIQTLTGLFKGDKCHSLVGKPKIFIIQACRGNQHDVPVIPLDVVDNQTEKLDTNITEVDAASVYTLPAGADFLMCYSVAEGYYSHRETVNGSWYIQDLCEMLGKYGSSLEFTELLTLVNRKVSQRRVDFCKDPSAIGKKQVPCFASMLTKKLHFFPKSN

SEQ ID NO: 60 - protein, 143 aa, Homo sapiens
MTETDAFYKREMFDPAEKYKMDHRRRGIALIFNHERFFWHLTLPERRGTCADRDNLTRRFSDLGFEVKCFNDLKAEELLLKIHEVSTVSHADADCFVCVFLSHGEGNHIYAYDAKIEIQTLTGLFKGDKCHSLVGKPKIFIIQ

SEQ ID NO: 61 - protein, 80 aa, Homo sapiens
MSSASGLRRGHPAGGEENMTETDAFYKREMFDPAEKYKMDHRRRGIALIFNHERFFWHLTLPERRGTCADRDNLTRSVNC

SEQ ID NO: 62 - RNA, 19 nt, synthetic construct
  source 1..19: mol_type = other RNA
caacataactgaggtggat

SEQ ID NO: 63 - RNA, 19 nt, synthetic construct
  source 1..19: mol_type = other RNA
catggtacattcaagattt

SEQ ID NO: 64 - RNA, 19 nt, synthetic construct
  source 1..19: mol_type = other RNA
aaatatggctcctccttag

SEQ ID NO: 65 - RNA, 19 nt, synthetic construct
  source 1..19: mol_type = other RNA
gttcaaaggagacaagtgt